SEQUENCE LISTING (54 sequences)

SEQ ID NO: 1 - DNA, 1998 bp, Araneus diadematus
gcgcatatgcatcatcatcaccatcaccaccatcatcatagcagcggtagcagcctggaagttctgtttcagggtccggcacgtgcaggtagcggtcagcagggtccgggtcagcaaggtcctggccagcagggaccaggtcaacagggtccgtatggtccgggtgcaagcgcagctgcagcagcagccggtggttatggtcctggttcaggtcagcagggaccgtcccagcagggtcctggacaacaaggaccgggtggtcagggtccttatggccctggtgccagtgccgcagcagctgccgcaggcggttatggtccaggtagtggccaacagggtcctggtggtcagggaccatacggtccgggtagcagtgcagcagccgcagcggctgggggtaatggtccgggaagtggacagcagggtgcagggcagcaaggcccaggacagcaaggtccgggtgcctcagcagcggcagccgcagctggcggatatggaccgggttcaggccaacaaggtccgggacaacagggtccaggcggacaaggtccgtatggcccaggcgcatcagccgctgctgccgcagcgggtggctatggaccaggcagtggtcaaggtccaggtcaacaaggccctggtggccaaggtccgtacggtcctggtgcgtcagccgcagcagcagctgcgggtggatacggtccaggctcaggccagcaaggccctgggcaacaagggcctggtcaacagggacctgggggacaaggcccttacggaccgggtgcatctgcggcagcggctgcagcgggtggttacggaccgggatatggccagcaaggacctggtcagcaaggtccaggcggtcagggaccgtatggaccgggtgcgtcagcagcaagcgcagccagtggtggttatggcccaggatctgggcagcagggtccaggccaacagggacctggcggacagggtccttatgggccaggggcatctgcagcggctgctgctgccggtggatatgggccaggtagcggacaacaagggccaggccagcagggtcctgggcagcagggtcctggtcagcaaggcccaggtggacaaggcccttatggtccaggtgcgagtgcagccgcagcggcagcgggtggatatggccctggaagcggtcagcaaggaccgggacaacaaggcccaggtcagcaaggaccagggcaacagggaccgggacagcagggacctggccaacaaggccctggacagcagggaccgggacagcaagggccaggacaacagggtccaggtggccagggtgcctatggtcctggcgcttcagcagcagcgggtgcagcagggggatatggtcctggctctgggcaacaaggcccaggccaacaaggtccaggccagcaagggccaggacagcagggacctggtcaacagggaccgggtcaacaaggtccgggacagcaagggcctgggcagcaaggaccttatggacctggcgcaagcgctgcagctgccgcagcaggcggttacggtccgggatccggtcaacaaggacctggccaacagggtcctggccagcaaggtccgggtggccaaggaccgtatgggccaggtgcagccagcgcagccgttagcgttggtggctatggtccgcagagcagcagcgttccggttgccagcgcagttgcaagccgtctgagcagtccggcagcaagcagtcgtgttagcagcgcagttagcagcctggttagcagcggtccgaccaaacatgcagcactgagcaataccattagcagcgttgttagccaggttagcgcaagcaatccgggtctgagcggttgtgatgttctggttcaggcactgctggaagttgttagcgcactggtgagcattctgggtagcagcagcattggtcagattaactatggtgccagcgcacagtatacccagatggttggtcagagcgttgcacaggcactggcataagaattcgcg

SEQ ID NO: 2 - protein, 660 aa, Araneus diadematus
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGPGQQGPGQQGPGQQGPGQQGPGQQGPGGQGAYGPGASAAAGAAGGYGPGSGQQGPGQQGPGQQGPGQQGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 3 - DNA, 2457 bp, Araneus diadematus
gcgccatatgcatcatcatcaccatcaccaccatcatcatagcagcggtagcagcctggaagttctgtttcagggtccggcacgtgcaggtagcggtcagcagggtccgggtcagcaaggtcctggccagcagggaccaggtcaacagggtccgtatggtccgggtgcaagcgcagctgcagcagcagccggtggttatggtcctggttcaggtcagcagggaccgtcccagcagggtcctggacaacaaggaccgggtggtcagggtccttatggccctggtgccagtgccgcagcagctgccgcaggcggttatggtccaggtagtggccagggtcgtggtgatagtccgcagggtccaggcggtcagggtccatacggtccaggtagcagtgcagcagccgcagcagcgggtggcaacggtcctggtagtggacagcagggtgcaggccaacaaggcccaggccagcaaggtccgggtgcctcagcagcagcggcagcggcagggggttatggaccgggttctggccaacagggtcgcggtgattcaccgggtccgggacaacagggtccaggtggccaaggtccgtatggcccaggcgcatcagctgctgcagcggctgcgggtggctatggcccaggttctggtcagggacgtggcgatagccctggtcctgggcagcaaggccctggtggccaaggcccttacggtccgggtgcttctgctgcggcagcagctgctggcggatatggtccgggaagcggtcagcaaggcccaggacaacaaggccctggtcaacagggacctggcggacagggaccgtacggacctggtgcatcagcggcagctgcggcagccggtggatacggaccgggttatggacagcaaggtcgtggcgacagtccgggaccgggacagcaaggaccaggcggacaaggcccatacggtcctggcgcaagtgcagcaagcgcagcaagtggtggttatggaccaggcagcggacagcagggacgtggtgactctccgggtccagggcagcagggaccgggtggccagggaccgtatggtccaggtgcttcagcagccgcagccgcagctggcggttacggacctggcagtggtcaacaagggccaggacagcagggacctggacagcagggtccagggcaacagggacctggcggtcaaggtccttatggaccgggtgcatctgccgcagcggcagctgcgggtggatatggtcctggcagtgggcaacagggtccgggtcaacagggaccgggacaacaaggtccaggacagcaaggccgtggtgatagcccaggcccaggccaacagggaccgggtcagcaagggccaggtcaacaaggtccgggtcaacaaggcccaggtcagcagggtccagggggacagggtgcttatggaccaggtgcgagcgcagcagcaggggctgcagggggatacggaccaggctcagggcaacaaggaccagggcaacaagggcctgggcagcaaggaccaggccagcagggtcgtggcgattctccaggccctgggcaacaaggtcctggccaacaaggtccgggacagcagggtccgggacagcaaggaccgtatggtccgggtgccagtgcggcagccgcagcggctgggggatacggtcctggatctggccagcaagggcctggtcagcaaggccctgggcaacagggtccagggggtcaaggaccttacggaccaggcgcagccagcgcagcagttagcgttggtggctatggtccgcagagcagcagcgttccggttgccagcgcagttgcaagccgtctgagcagtccggcagcaagcagccgtgttagcagcgcagttagcagcctggtgagcagcggtccgaccaaacatgcagcactgagcaataccattagcagcgttgttagccaggttagcgcaagcaatccgggtctgagcggttgtgatgttctggttcaggcactgctggaagttgttagcgcactggttagcattctgggtagcagcagcattggtcagattaactatggtgccagcgcacagtatacccagatggttggtcagagcgttgcacaggcactggcataatcccacagccgccagttccgctggcggcattttaactttctttaatgaagccggaaaaatcctaaattcatttaatatttatctttttaccgtttcgcttaccccggtcgaacgtcaacttacgtcatttttccgcccaacagtaatataatcaaacaaattaatcccgcaacataacaccagtaaaatcaataattttctctaagtcacttattcctcaggtaattgttaatatatccagaatgttcctcaaaatatattttccctctatcttctcgttgcgcttaatttgactaattctcattagcgactaattttaatgagtgtcgacgaattcgcgc

SEQ ID NO: 4 - protein, 702 aa, Araneus diadematus
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQGRGDSPQGPGGQGPYGPGSSAAAAAAGGNGPGSGQQGAGQQGPGQQGPGASAAAAAAGGYGPGSGQQGRGDSPGPGQQGPGGQGPYGPGASAAAAAAGGYGPGSGQGRGDSPGPGQQGPGGQGPYGPGASAAAAAAGGYGPGSGQQGPGQQGPGQQGPGGQGPYGPGASAAAAAAGGYGPGYGQQGRGDSPGPGQQGPGGQGPYGPGASAASAASGGYGPGSGQQGRGDSPGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGRGDSPGPGQQGPGQQGPGQQGPGQQGPGQQGPGGQGAYGPGASAAAGAAGGYGPGSGQQGPGQQGPGQQGPGQQGRGDSPGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 5 - DNA, 2541 bp, Araneus diadematus
gcgccatatgcatcatcatcaccatcaccaccatcatcatagcagcggtagcagcctggaagttctgtttcagggtccggcacgtgcaggtagcggtcagcagggtccgggtcagcaaggtcctggccagcagggaccaggtcaacagggtccgtatggtccgggtgcaagcgcagctgcagcagcagccggtggttatggtcctggttcaggtcagcagggaccgtcccagcagggtcctggacaacaaggaccgggcggtcagggtccttatggccctggtgccagtgccgcagcagctgccgcaggcggttatggtccaggtagtggtcaggttaccggtcgtggtgatagtccggcaagccagggtcctggtggtcagggaccatacggtccgggtagcagtgcagcagccgcagcggctgggggtaatggtcctggtagcggacagcaaggcgcaggacagcagggacctggccagcaaggccctggtgcatctgcagcggctgcagcagctggcggatatggaccgggtagtggccagcaggttacaggccgtggcgattcaccggcaagcggtccgggtcaacagggacctggtggccaaggaccgtatggcccaggtgcatcagctgcggcagcggcagcgggtggctatggaccaggctctggtcaggtgacaggtcgcggagattctccggcatcaggtccgggacagcagggtccaggcggtcaaggtccgtatggacctggcgcttcagcagcagcggcagctgcgggtggatatggccctggcagcggtcaacaaggaccagggcagcaaggaccgggacaacaaggcccaggcggacagggaccatacgggcctggtgcgtcagccgcagccgctgctgccggtggttacggaccgggttatggtcaacaggtgaccggacgcggtgatagccctgccagcggtccaggccaacaagggcctgggggtcaaggcccttacggtcctggtgcatcagcggcaagcgcagcaagtggtggctatggtccgggatctggacagcaggttacgggtcggggagatagccctgcgtcaggtccaggacagcaaggcccaggtggccaaggcccttatggaccgggtgcgtctgcagccgcagctgcagcgggtggatacggtccgggaagcggacaacagggtcctgggcaacagggtccaggccagcagggaccgggacagcagggaccaggtggccagggtccttacggtccaggcgcatctgctgccgctgcagccgcagggggttacggtccgggatcaggccagcaagggccaggccaacaaggtccaggacaacagggtccgggtcaacaagttaccggtcgcggtgactcaccagccagtggccctggacagcaagggcctggtcaacagggacctggacagcagggtcctggtcagcagggtccgggacaacagggacctggcggacagggtgcgtatgggccaggcgctagtgcagcagcgggtgcagctggcggttacggtcctggctctggccaacaaggacctggtcagcaaggcccagggcagcagggtccagggcagcaagttacgggtcgtggcgacagcccagcaagtggccctggccaacagggtcctggccaacaaggtcctggacagcaaggaccgggtcagcagggtccttacggaccgggtgcttcggcagcagctgcagcagccggtggctacggacctggcagtggacagcaaggtccagggcaacaaggccctggccagcaaggtccaggtgggcagggaccgtacggacctggtgcagcctcagcagcagttagcgttggtggttatggaccgcagagcagcagcgttccggttgccagcgcagttgcaagccgtctgagcagtccggcagcaagcagccgtgttagcagcgcagttagcagcctggtgagcagcggtccgaccaaacatgcagcactgagcaataccattagcagcgttgttagccaggttagcgcaagcaatccgggtctgagcggttgtgatgttctggttcaggcactgctggaagttgttagcgcactggttagcattctgggtagcagcagcattggtcagattaactatggtgccagcgcacagtatacccagatggttggtcagagcgttgcacaggcactggcataatcccacagccgccagttccgctggcggcattttaactttctttaatgaagccggaaaaatcctaaattcatttaatatttatctttttaccgtttcgcttaccccggtcgaacgtcaacttacgtcatttttccgcccaacagtaatataatcaaacaaattaatcccgcaacataacaccagtaaaatcaataattttctctaagtcacttattcctcaggtaattgttaatatatccagaatgttcctcaaaatatattttccctctatcttctcgttgcgcttaatttgactaattctcattagcgactaattttaatgagtgtcgacgaattcgcgc

SEQ ID NO: 6 - protein, 730 aa, Araneus diadematus
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQVTGRGDSPASQGPGGQGPYGPGSSAAAAAAGGNGPGSGQQGAGQQGPGQQGPGASAAAAAAGGYGPGSGQQVTGRGDSPASGPGQQGPGGQGPYGPGASAAAAAAGGYGPGSGQVTGRGDSPASGPGQQGPGGQGPYGPGASAAAAAAGGYGPGSGQQGPGQQGPGQQGPGGQGPYGPGASAAAAAAGGYGPGYGQQVTGRGDSPASGPGQQGPGGQGPYGPGASAASAASGGYGPGSGQQVTGRGDSPASGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQVTGRGDSPASGPGQQGPGQQGPGQQGPGQQGPGQQGPGGQGAYGPGASAAAGAAGGYGPGSGQQGPGQQGPGQQGPGQQVTGRGDSPASGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 7 - protein, 9 aa, Araneus diadematus
GASAAAAAA

SEQ ID NO: 8 - protein, 9 aa, Araneus diadematus
GSSAAAAAA

SEQ ID NO: 9 - protein, 9 aa, Araneus diadematus
GASAASAAS

SEQ ID NO: 10 - protein, 9 aa, Araneus diadematus
GASAAAGAA

SEQ ID NO: 11 - protein, 11 aa, Araneus diadematus
GGYGPGSGQQG

SEQ ID NO: 12 - protein, 11 aa, Araneus diadematus
GGNGPGSGQQG

SEQ ID NO: 13 - protein, 11 aa, Araneus diadematus
GGYGPGYGQQG

SEQ ID NO: 14 - protein, 10 aa, Araneus diadematus
GGYGPGSGQG

SEQ ID NO: 15 - protein, 5 aa, Araneus diadematus
PGQQG

SEQ ID NO: 16 - protein, 5 aa, Araneus diadematus
AGQQG

SEQ ID NO: 17 - protein, 5 aa, Araneus diadematus
SGQQG

SEQ ID NO: 18 - protein, 5 aa, Araneus diadematus
PSQQG

SEQ ID NO: 19 - protein, 5 aa, Araneus diadematus
PGGQG

SEQ ID NO: 20 - protein, 4 aa, Araneus diadematus
PYGP

SEQ ID NO: 21 - protein, 4 aa, Araneus diadematus
AYGP

SEQ ID NO: 22 - protein, 6 aa, Homo sapiens
GRGDSP

SEQ ID NO: 23 - protein, 10 aa, Homo sapiens
VTGRGDSPAS

SEQ ID NO: 24 - protein, 11 aa, Homo sapiens
PQVTRGDVFTM

SEQ ID NO: 25 - protein, 7 aa, Homo sapiens
VTRGDVF

SEQ ID NO: 26 - DNA, 2190 bp, Araneus diadematus
atgcatcatcaccaccatcaccatcatcatcacagcagcggtagcagcgcacgtgcaggtagcggtcagcagggtccgggtcagcaaggccctggtcaacagggacctggccagcagggaccgtatggtccgggtgcaagtgcagcagcagccgcagccggtggttatggtcctggttcaggccagcaaggtccgagccaacagggtccaggacaacaaggaccgggtggtcagggtccttatggccctggtgcatcagctgcggcagcggcagcaggcggatatggaccgggtagtggtcagccgcaggttacccgtggtgatgtttttaccatgcagggtcctggtggccaaggtccgtacggtcctggtagtagcgctgctgccgcagcagcgggtggtaacggtccgggtagcggacaacagggagccggtcaacaagggcccgggcaacaaggtcctggcgcaagcgcagccgctgctgctgcgggtggctatggcccaggatcaggtcagcagcctcaggtgacacgcggagatgtgtttacaatgggtcccggacagcagggcccaggcggacaaggcccttatggtcccggagctagcgcagctgcagcggctgccggaggatacggtccaggctctggccagccgcaagtaacacgtggcgacgtgttcaccatgggaccggggcagcaagggccaggtggtcaaggaccatacggaccaggtgccagtgcggctgctgcagccgccggtggctacggacctggaagcggccaacaaggcccaggacagcaggggcctggacaacagggtcccggtggacagggcccatacggcccgggtgcgagcgcagcggccgctgccgctggcggttacggacccggttatggccagcaaccacaggtgacgcgtggggacgtatttacgatggggccaggccaacagggccctggagggcagggaccttacgggccaggcgcatcagcagccagcgcagcaagtggaggttatggaccaggcagcggtcaacagccacaagtcactcgcggtgacgtctttactatgggacctggtcaacaaggtccgggaggacaaggaccgtatggccctggagcctctgccgcagccgcagcagctggcggatacggtcctggcagcggtcagcaaggaccaggccaacaagggccgggtcagcagggaccaggacagcaaggtcccggaggtcaagggccttacggtccgggtgcttcagccgctgcagccgcagcgggagggtacggaccgggttcaggtcaacagggcccaggtcaacagggtcccgggcagcaggggcctggtcagcagccgcaagttactagaggtgatgtgttcacgatgggccctggccagcaaggacccgggcagcaaggacctggacaacaaggcccagggcaacagggaccgggtcaacaaggacctggcggacagggtgcctacggtcccggggcctccgcggctgcgggagctgctggagggtatgggcctggcagtggacagcagggtccaggccagcagggtcctggccaacaagggcctggacaacaaccgcaggttacacgcggtgacgtattcacaatgggcccagggcagcagggtccagggcaacagggcccaggacagcaaggcccaggtcagcaaggtccgtatggtccaggggcatcagcggctgctgcggctgctgggggttatggtccgggatctgggcaacaaggacccggccaacaggggccaggccagcaaggacctggaggacaaggtccttatggaccgggtgcagcaagcgcagcagttagcgttggtggctatggtccgcagagcagcagcgttccggttgccagtgcagttgcaagccgtctgagcagtccggcagcaagcagtcgtgtgagcagcgcagttagcagcctggttagcagtggtccgaccaaacatgcagcactgagcaataccattagcagcgttgttagccaggttagcgcaagcaatccgggtctgagcggttgtgatgttctggtgcaggcactgctggaagttgttagcgcactggtgagcattctgggtagcagcagcattggtcagattaactatggtgccagcgcacagtatacccagatggttggtcagagcgttgcacaggcactggcataa

SEQ ID NO: 27 - protein, 729 aa, Araneus diadematus
MHHHHHHHHHHSSGSSARAGSGQQGPGQQGPGQQGPGQQGPYGPGASAAAAAAGGYGPGSGQQGPSQQGPGQQGPGGQGPYGPGASAAAAAAGGYGPGSGQPQVTRGDVFTMQGPGGQGPYGPGSSAAAAAAGGNGPGSGQQGAGQQGPGQQGPGASAAAAAAGGYGPGSGQQPQVTRGDVFTMGPGQQGPGGQGPYGPGASAAAAAAGGYGPGSGQPQVTRGDVFTMGPGQQGPGGQGPYGPGASAAAAAAGGYGPGSGQQGPGQQGPGQQGPGGQGPYGPGASAAAAAAGGYGPGYGQQPQVTRGDVFTMGPGQQGPGGQGPYGPGASAASAASGGYGPGSGQQPQVTRGDVFTMGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQPQVTRGDVFTMGPGQQGPGQQGPGQQGPGQQGPGQQGPGGQGAYGPGASAAAGAAGGYGPGSGQQGPGQQGPGQQGPGQQPQVTRGDVFTMGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 28 - DNA, 2106 bp, Araneus diadematus
atgcatcatcaccaccatcaccatcatcatcacagcagcggtagcagcgcacgtgcaggtagcggtcagcagggtccgggtcagcaaggccctggtcaacagggacctggccagcagggaccgtatggtccgggtgcaagtgcagcagcagccgcagccggtggttatggtcctggttcaggccagcaaggtccgagccaacagggtccaggacaacaaggaccgggtggtcagggtccttatggccctggtgcatcagctgcggcagcggcagcaggcggatatggaccgggtagtggtcaggttacccgtggtgatgtttttcaaggtcctggtggccaaggtccgtacggtcctggtagtagcgctgctgccgcagcagcgggtggtaacggtccgggtagcggacaacagggagccggtcaacaagggcccgggcaacaaggtccaggtgcctcagcagccgctgcagcggcaggcggttatggcccaggatcaggtcagcaggttacacgcggagatgtgtttggtcccggacagcagggcccaggtggtcaaggaccttacggaccaggtgccagtgcggctgctgcagctgccggtggatacggacctggctctggtcaagtgactcgcggtgacgtttttgggcctggccagcaagggcctggcggtcagggtccatacggaccgggtgcttccgcagcggcggctgcagcgggaggttatgggccaggtagtgggcagcaaggacctggacagcaaggtcccggacaacaaggccctggcggacagggcccgtatggcccaggcgcttcagccgcagctgcagcagccggaggctacggtccaggttatggtcagcaagtgacccgtggcgacgtatttggtccggggcaacaggggcctggaggccaaggcccttatggacctggcgcaagcgcagccagcgcagcaagtggtggctatggaccaggcagtggccaacaggtgacacgtggtgacgtgttcggaccaggccaacagggaccaggcggtcaaggcccctatggtcccggtgcttctgcggctgccgctgccgccggcgggtatgggcctggaagcggccaacaaggtcctgggcagcaggggccagggcagcagggtcctggccaacaagggccagggggacaaggaccttatggtccaggggcatctgcagctgctgctgctgcgggagggtacggtccgggaagtggtcagcaggggcccggacagcaaggaccaggccagcagggccctggacagcaggtaacacgtggggatgttttcggccctggtcagcaaggcccgggacagcagggtccgggtcaacagggtccgggacagcaggggcctggacaacaaggtcctggaggccagggtgcctatggtccgggagcttcagcagcagcgggagctgcgggtggttatggacccggaagcgggcaacagggccccggacaacagggtcccggccagcaagggccgggtcagcaggttacgcgtggggacgtttttggtccaggtcagcagggaccagggcaacaaggcccaggtcagcaaggtccaggccagcaaggtccttatggtcctggtgccagcgcagctgctgcggcagctggcggctacggccctggttcgggtcaacaaggaccaggacaacagggcccagggcaacaggggccaggcggacagggtccgtacggtcccggtgcagcaagcgcagcagttagcgttggcggttatggtccgcagagcagcagcgttccggttgcaagcgcagttgcaagccgtctgagcagtccggcagcaagcagtcgtgtgagcagcgcagttagcagcctggttagcagcggtccgaccaaacatgcagcactgagcaataccattagcagcgttgttagccaggttagcgcaagcaatccgggtctgagcggttgtgatgttctggtgcaggcactgctggaagttgttagcgcactggtgagcattctgggtagcagcagcattggtcagattaactatggtgcgagcgcacagtatacccagatggttggtcagagcgttgcacaggcactggcataa

SEQ ID NO: 29 - protein, 701 aa, Araneus diadematus
MHHHHHHHHHHSSGSSARAGSGQQGPGQQGPGQQGPGQQGPYGPGASAAAAAAGGYGPGSGQQGPSQQGPGQQGPGGQGPYGPGASAAAAAAGGYGPGSGQVTRGDVFQGPGGQGPYGPGSSAAAAAAGGNGPGSGQQGAGQQGPGQQGPGASAAAAAAGGYGPGSGQQVTRGDVFGPGQQGPGGQGPYGPGASAAAAAAGGYGPGSGQVTRGDVFGPGQQGPGGQGPYGPGASAAAAAAGGYGPGSGQQGPGQQGPGQQGPGGQGPYGPGASAAAAAAGGYGPGYGQQVTRGDVFGPGQQGPGGQGPYGPGASAASAASGGYGPGSGQQVTRGDVFGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQVTRGDVFGPGQQGPGQQGPGQQGPGQQGPGQQGPGGQGAYGPGASAAAGAAGGYGPGSGQQGPGQQGPGQQGPGQQVTRGDVFGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 30 - protein, 6 aa, Homo sapiens
GRGDNP

SEQ ID NO: 31 - protein, 13 aa, Homo sapiens
GAAGRGDSPAAGY

SEQ ID NO: 32 - protein, 25 aa, Homo sapiens
GPGQQGPGQQGPGQQVTGRGDSPAS

SEQ ID NO: 33 - protein, 20 aa, Homo sapiens
VTGRGDSPASVTGRGDSPAS

SEQ ID NO: 34 - protein, 13 aa, Homo sapiens
GKKQRFRHRNRKG

SEQ ID NO: 35 - DNA, 2109 bp, Araneus diadematus
atgcatcatcaccaccatcaccatcatcatcacagcagcggtagcagcctggaagttctgtttcagggtccggcacgtgcaggtagcggtcagcagggtccgggtcagcaaggccctggtcaacagggacctggccagcagggaccgtatggtccgggtgcaagtgcagcagcagccgcagccggtggttatggtcctggttcaggccagcaaggtccgagccaacagggtccaggacaacaaggaccgggtggtcagggtccttatggccctggtgcatcagctgcggcagcggcagcaggcggatatggaccgggtagtggccagggtcgtggtgataatccgcagggtcctggcggacagggtccgtacggtcctggtagtagcgctgctgccgcagcagcgggtggtaacggtccgggaagtggacagcagggtgctgggcaacagggcccaggtcagcaaggtccaggtgcatcagccgctgctgctgcagccggtggctacggaccaggcagtggtcaacaaggccgtggcgacaatccgggaccggggcaacaaggtcctggtggccaaggcccttacggtccaggcgctagtgcagccgcggctgcagctggaggatatggtccaggctcaggtcagggacgcggagataatccgggtcctgggcagcaaggaccaggcggtcaaggaccatacggacctggtgcctccgcagcggctgctgcggctggcggttacggccctggatctggccaacaagggcctggacagcaaggtcccggacagcagggacccggtggccaaggtccgtatggaccaggtgccagcgctgccgcggccgctgcgggagggtacggaccgggttatggacaacaggggagaggcgataaccctggaccagggcagcaggggcctggagggcagggcccctatggacccggagcctcagctgcaagcgcagcaagcggtgggtatggcccaggaagtggccaacagggaagaggtgacaatcctggtccgggacaacagggccctggaggtcaagggccttatggtcccggtgcttcagcagctgcagcagctgccggaggatacgggcctgggagcggtcaacaagggccagggcaacagggtcctggtcaacagggtccgggccaacaaggtccaggcggacagggaccttatggcccaggcgcttctgcagcagcggcagctgcaggcggttacggtcccggtagcggacagcaaggcccaggccaacaaggccccggtcagcagggcccaggacaacagggtcgcggtgataaccctggtccaggtcaacaaggccctggccagcaagggccgggtcaacaaggaccgggacaacaaggaccaggtcagcagggaccgggtggacagggtgcatacggtccgggtgcctctgctgcagctggtgcagcgggtggatatggccctgggtctggacagcaggggccaggccagcagggccctggccaacaaggacctgggcaacagggacgtggcgataacccaggcccgggccaacagggacccggtcaacaaggtcccgggcagcaaggacctggtcagcaaggtccgtatggccctggcgcaagcgcagccgctgcagccgcaggaggctatggacctggatccggtcagcaaggaccggggcagcagggccccggacagcaagggcctggaggacaaggtccttacggaccgggagcagccagcgcagcagttagcgttggaggctacggtccgcagagcagcagcgttccggttgcgagcgcagttgcaagccgtctgagcagtccggcagcaagcagtcgtgtgagcagcgcagttagcagcctggttagcagcggtccgaccaaacatgcagcactgagcaataccattagcagcgttgttagccaggttagcgcaagcaatccaggtctgagcggttgtgatgttctggtgcaggcactgctggaagttgttagcgcactggtgagcattctgggtagcagcagcattggtcagattaactatggtgccagtgcacagtatacccagatggttggtcagagcgttgcacaggcactggcataa

SEQ ID NO: 36 - protein, 702 aa, Araneus diadematus
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQGRGDNPQGPGGQGPYGPGSSAAAAAAGGNGPGSGQQGAGQQGPGQQGPGASAAAAAAGGYGPGSGQQGRGDNPGPGQQGPGGQGPYGPGASAAAAAAGGYGPGSGQGRGDNPGPGQQGPGGQGPYGPGASAAAAAAGGYGPGSGQQGPGQQGPGQQGPGGQGPYGPGASAAAAAAGGYGPGYGQQGRGDNPGPGQQGPGGQGPYGPGASAASAASGGYGPGSGQQGRGDNPGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGRGDNPGPGQQGPGQQGPGQQGPGQQGPGQQGPGGQGAYGPGASAAAGAAGGYGPGSGQQGPGQQGPGQQGPGQQGRGDNPGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 37 - DNA, 1749 bp, Araneus diadematus
atgcatcatcaccaccatcaccatcatcatcacagcagcggtagcagcgcacgtgcaggtagcggtcagcagggtccgggtcagcaaggccctggtcaacagggacctggccagcagggaccgtatggtccgggtgcaagtgcagcagcagccgcagccggtggttatggtcctggttcaggccagcaaggtccgagccaacagggtccaggacaacaaggaccgggtggtcagggtccttatggccctggtgcatcagctgcggcagcggcagcaggcggatatggaccgggttcaggtcagggtgcagcaggtcgtggtgatagtccggcagccggttatggtagctctgccgctgctgcagcagcgggtggtaacggtccgggtagtggacagcaaggtgcaggtcaacaagggcccgggcaacaaggtcctggcgcatctgcagctgccgccgctgcgggaggatatggtccaggctctgggcagcagggcgctgccggacgtggtgattcaccagccgcaggttatggtgccagtgccgctgccgcggccgctggcggttatggcccaggtagtggtcaaggtgctgctggccgtggcgatagccctgcggctggttatggcgcaagcgcagctgcagcggcggctggaggctacggtcctggaagcggacaacaggggcctggacagcaaggcccaggacagcagggtccaggtggccaaggtccgtatggcccaggcgcttcagccgcagctgctgctgccggtggctatggaccaggttatgggcaacagggtgccgctggtcgcggagattctcctgcagcaggctatggcgcatcagcggcatcagcagcaagcggaggttacggaccgggatctggccaacaaggcgcagcgggacgcggtgatagcccagctgcgggatatggtgcgagcgcagccgctgcagccgccggtggatacggcccaggaagtgggcagcaaggacccggtcagcaaggaccgggacaacaagggccaggccaacagggaccaggcggacagggtccgtacggaccaggtgctagcgctgcggccgcagcggctggggggtatggacctggcagtggccaacagggtgcggcaggacgaggcgattcaccggctgctggctacggtgcgtctgctgccgctggggctgcaggcggttacggtccagggtcaggtcaacagggggcagccggtagaggtgattcccctgcagcgggttacggtgcctcagccgccgcggctgccgcaggggggtacggccctggatctggtcagcaaggtcctggccagcaaggtccagggcaacaaggccctggtggccaaggaccttacggtcctggtgcagcaagcgcagcagttagcgttggtggctatggtccgcagagcagcagcgttccggttgccagcgcagttgcaagccgtctgagttcaccggcagcaagcagtcgtgttagcagtgcagttagcagcctggttagcagcggtccgaccaaacatgcagcactgagcaataccattagcagcgttgttagccaggttagcgcaagcaatccgggtctgagcggttgtgatgttctggtgcaggcactgctggaagttgttagcgcactggtgagcattctgggtagcagcagcattggtcagattaactatggtgcgtcagcacagtatacccagatggttggtcagagcgttgcacaggcactggcataa

SEQ ID NO: 38 - protein, 582 aa, Araneus diadematus
MHHHHHHHHHHSSGSSARAGSGQQGPGQQGPGQQGPGQQGPYGPGASAAAAAAGGYGPGSGQQGPSQQGPGQQGPGGQGPYGPGASAAAAAAGGYGPGSGQGAAGRGDSPAAGYGSSAAAAAAGGNGPGSGQQGAGQQGPGQQGPGASAAAAAAGGYGPGSGQQGAAGRGDSPAAGYGASAAAAAAGGYGPGSGQGAAGRGDSPAAGYGASAAAAAAGGYGPGSGQQGPGQQGPGQQGPGGQGPYGPGASAAAAAAGGYGPGYGQQGAAGRGDSPAAGYGASAASAASGGYGPGSGQQGAAGRGDSPAAGYGASAAAAAAGGYGPGSGQQGPGQQGPGQQGPGQQGPGGQGPYGPGASAAAAAAGGYGPGSGQQGAAGRGDSPAAGYGASAAAGAAGGYGPGSGQQGAAGRGDSPAAGYGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 39 - DNA, 2394 bp, Araneus diadematus
atgcatcatcaccaccatcaccatcatcatcacagcagcggtagcagcgcacgtgcaggtagcggtcagcagggtccgggtcagcaaggccctggtcaacagggacctggccagcagggaccgtatggtccgggtgcaagtgcagcagcagccgcagccggtggttatggtcctggttcaggccagcaaggtccgagccaacagggtccaggacaacaaggaccgggtggtcagggtccttatggccctggtgcatcagctgcggcagcggcagcaggcggatatggaccgggtagtggtcaaggaccagggcagcagggccccgggcaacaagggcccggacagcaggttaccggtcgtggtgatagtccggcaagccagggtcctggtggccaaggtccgtacggtcctggtagtagcgctgctgccgcagcagcgggtggtaacggtccgggtagcggacaacagggagccggtcaacaaggtcccggccaacaaggcccaggtgcatctgcagccgctgcagcggcaggcggttatggcccaggatcaggtcagcaggggccaggacagcagggtcccggccaacagggaccgggccaacaggtgacaggtcgcggtgattcaccggcaagcggtcctgggcagcagggtccaggtggtcagggaccatacggaccgggtgcctcagccgcagctgcagctgcgggtggctatggtccaggcagcggtcaaggtcctggacagcaagggccaggccaacaagggcctggtcagcaagtgacaggcagaggtgatagccctgcatcaggaccgggacaacaaggtcctggcggtcaaggaccgtatggcccaggcgcttcagccgctgcggctgctgctggcggttacggacctggcagcgggcaacagggtccaggtcagcagggaccaggtcaacaaggaccaggcggacaaggtccatacggaccaggcgcaagcgcagccgctgccgcagctggcggatacggtccaggttatgggcagcaaggacccggtcagcaaggtccagggcaacagggtccggggcagcaagttacaggtcgtggcgattctcctgcctcaggcccaggacaacaggggccagggggacagggcccttatggtcccggagccagcgcagcatcagcagcaagtggtgggtatgggccaggtagtggccaacaaggtccgggacagcagggacctggacagcaaggcccgggacagcaagtgactggccgtggggactcaccggcttcagggccgggtcaacagggcccaggaggccaaggaccttacgggccaggtgctagtgctgcggcggcagctgccggagggtatggacctggatctggacaacagggccctggacaacagggtccgggccaacaaggtcctggccagcaaggtccgggtggccaaggcccttatggaccgggtgcgtctgcggctgcagctgcagcgggtggatatggcccaggaagtgggcagcaaggcccaggccagcaggggcctggacaacaaggtccagggcagcaggtaacaggacgtggtgacagccctgcctctgggcctggccaacagggtcccggtcaacagggtcccgggcagcaggggcctggtcagcagggtcctggacagcagggcccaggtggccaaggtgcctatggtccaggtgcatcagcagcagcgggtgcagccggtggctatggaccgggaagcggacagcaaggtcctggtcaacaaggtcccggtcagcaaggaccgggacagcaggttacgggacgcggagattcaccagcatctggaccgggccagcaaggacccggacaacagggcccaggtcagcaaggtccaggccagcaaggaccttatggtcctggcgcatctgccgcagccgcagcagccggaggttacggacccggaagtggtcaacaagggccaggacagcaaggccccggtcagcagggccctggtggacagggtccgtatgggccaggggcagcaagcgcagcagttagcgttggcggttatggtccgcagagcagcagcgttccggttgcaagcgcagttgcaagccgtctgagcagtccggcagcatcaagccgtgttagcagtgcagttagcagcctggttagctcaggtccgaccaaacatgcagcactgagcaataccattagcagcgttgtgagccaggttagcgcaagcaatccgggtctgagcggttgtgatgttctggtgcaggcactgctggaagttgttagcgcactggtgagcattctgggtagcagcagcattggtcagattaactatggtgccagcgcacagtatacccagatggttggtcagagcgttgcacaggcactggcataa

SEQ ID NO: 40 - protein, 797 aa, Araneus diadematus
MHHHHHHHHHHSSGSSARAGSGQQGPGQQGPGQQGPGQQGPYGPGASAAAAAAGGYGPGSGQQGPSQQGPGQQGPGGQGPYGPGASAAAAAAGGYGPGSGQGPGQQGPGQQGPGQQVTGRGDSPASQGPGGQGPYGPGSSAAAAAAGGNGPGSGQQGAGQQGPGQQGPGASAAAAAAGGYGPGSGQQGPGQQGPGQQGPGQQVTGRGDSPASGPGQQGPGGQGPYGPGASAAAAAAGGYGPGSGQGPGQQGPGQQGPGQQVTGRGDSPASGPGQQGPGGQGPYGPGASAAAAAAGGYGPGSGQQGPGQQGPGQQGPGGQGPYGPGASAAAAAAGGYGPGYGQQGPGQQGPGQQGPGQQVTGRGDSPASGPGQQGPGGQGPYGPGASAASAASGGYGPGSGQQGPGQQGPGQQGPGQQVTGRGDSPASGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQVTGRGDSPASGPGQQGPGQQGPGQQGPGQQGPGQQGPGGQGAYGPGASAAAGAAGGYGPGSGQQGPGQQGPGQQGPGQQVTGRGDSPASGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 41 - DNA, 2196 bp, Araneus diadematus
atgcatcatcaccaccatcaccatcatcatcacagcagcggtagcagcctggaagttctgtttcagggtccggcacgtgcaggtagcggtcagcagggtccgggtcagcaaggccctggtcaacagggacctggccagcagggaccgtatggtccgggtgcaagtgcagcagcagccgcagccggtggttatggtcctggttcaggccagcaaggtcgtggtgatagtccgggtccgagccaacagggtccaggacaacaaggaccgggtggtcagccgtatggccctggtgcttctgccgctgctgcagcagcgggtggctatggaccgggtagtggtcagggtcgcggtgattcaccgcagggtcctggtggccagggtccttatggtccaggtagtagcgcagctgcagcggcagcaggcggtaacggtccgggaagtggccaacaaggccgtggcgattcaccgggtgcaggacagcagggcccagggcagcaggggcccggtgcatctgctgccgccgctgcggctggcggttatggcccaggatctggacagcaaggacgcggagacagcccaggaccggggcaacaaggtcctggcggtcaaggtccgtacggacctggtgcaagcgctgcggccgcagctgccggtggatacggaccaggatcaggtcagggacgtggcgatagcccaggccccggacagcaaggtccaggtggccaaggaccatacggcccaggtgccagtgccgcggctgctgctgcgggaggttacggtccaggcagcggacaacaggggagaggcgattctccaggcccaggccaacaagggcctggtcagcaaggaccaggcggacagggtccctacgggccaggcgcatctgccgcagcggctgcagccggagggtacggaccgggttatgggcaacaggggcgtggtgactcaccagggcccggtcaacaaggtccgggagggcaaggcccttatgggcctggtgcatcagcagcaagcgcagccagcggaggttatggacccggatcgggccaacagggaagaggtgatagccctggtccgggacagcagggacccggaggtcagggaccttatggccctggagccagcgctgccgcagcagcagctggaggatatggtccgggatccggacaacaaggtcggggtgactcaccgggtcctgggcagcagggtcctggccaacaaggtcccggacagcaggggcctggtggacaaggcccttacggaccgggagcctctgctgctgcagccgctgcaggcggatatggccctggcagcggccagcaaggaccgggacaacaggggccaggtcaacagggtcccgggcaacagggtaggggtgacagtccaggtccagggcagcaagggccaggacagcaaggtcctggccagcagggcccaggacaacagggccctggacaacaaggcccaggcggtcagggagcctatggtccaggggcttctgcagcagccggtgccgcaggcggttacgggcctggttctggacaacagggaccgggacagcaagggccgggccaacaaggacccggccaacaaggtagaggggattctcctggtccagggcaacagggacccggtcagcagggccctggacagcagggtccggggcaacagggaccgtatggcccaggcgcatcagcagctgcggcagccgcaggaggatacggtcctggctcggggcagcagggacggggagatagtcctggcccaggtcagcagggaccaggtcaacaagggccaggtgggcagggtccgtatgggcctggggcagcatctgcagcagttagcgttggtgggtatggtccgcagagcagcagcgttccggttgccagcgcagttgcaagccgtctgagcagtccggcagcaagcagtcgtgttagcagtgcagttagcagcctggttagcagcggtccgaccaaacatgcagcactgagcaataccattagcagcgttgttagccaggttagcgcaagcaatccgggtctgagcggttgtgatgttctggtgcaggcactgctggaagttgttagcgcactggtgagcattctgggtagcagcagcattggtcagattaactatggtgcgagcgcacagtatacccagatggttggtcagagcgttgcacaggcactggcataa

SEQ ID NO: 42 - protein, 731 aa, Araneus diadematus
MHHHHHHHHHHSSGSSLEVLFQGPARAGSGQQGPGQQGPGQQGPGQQGPYGPGASAAAAAAGGYGPGSGQQGRGDSPGPSQQGPGQQGPGGQPYGPGASAAAAAAGGYGPGSGQGRGDSPQGPGGQGPYGPGSSAAAAAAGGNGPGSGQQGRGDSPGAGQQGPGQQGPGASAAAAAAGGYGPGSGQQGRGDSPGPGQQGPGGQGPYGPGASAAAAAAGGYGPGSGQGRGDSPGPGQQGPGGQGPYGPGASAAAAAAGGYGPGSGQQGRGDSPGPGQQGPGQQGPGGQGPYGPGASAAAAAAGGYGPGYGQQGRGDSPGPGQQGPGGQGPYGPGASAASAASGGYGPGSGQQGRGDSPGPGQQGPGGQGPYGPGASAAAAAAGGYGPGSGQQGRGDSPGPGQQGPGQQGPGQQGPGGQGPYGPGASAAAAAAGGYGPGSGQQGPGQQGPGQQGPGQQGRGDSPGPGQQGPGQQGPGQQGPGQQGPGQQGPGGQGAYGPGASAAAGAAGGYGPGSGQQGPGQQGPGQQGPGQQGRGDSPGPGQQGPGQQGPGQQGPGQQGPYGPGASAAAAAAGGYGPGSGQQGRGDSPGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 43 - DNA, 1995 bp, Araneus diadematus
atgcatcatcaccaccatcaccatcatcatcacagcagcggtagcagcgcacgtgcaggtagcggtcagcagggtccgggtcagcaaggccctggtcaacagggacctggccagcagggaccgtatggtccgggtgcaagtgcagcagcagccgcagccggtggttatggtcctggttcaggccagcaaggtccgagccaacagggtccaggacaacaaggaccgggtggtcagggtccttatggccctggtgcatcagctgcggcagcggcagcaggcggatatggaccgggttctgggcaacaaggtcctggcggtcaaggcccttacggtccgggtagtagcgctgctgccgcagcagcgggtggtaatggcccaggcagtggacagcagggtgctggacaacagggcccaggtcagcaaggtccaggtgcatcagccgctgctgctgcagccggtggctacggaccaggttctggtcagcaaggaccgggacagcaggggccaggtggccaaggtccgtatggaccaggagccagtgctgctgcggctgccgctggcggttacggtccaggatcaggtcaaggacccggccaacaaggccccggtggacaaggaccatacggtcctggtgcctccgcagccgcggctgcagcgggaggatatgggcctggaagcgggcagcagggacccggacaacagggtcccgggcaacaggggcctggcggacagggtccctacggacctggcgctagtgcagcggctgcggccgctggaggctatggacctggctacggtcaacaagggccagggcagcagggtcctggaggccagggaccttacggcccaggcgcaagcgctgcaagcgcagcatcaggtggttacggccctgggagcggacagcaaggcccagggcaacaaggcccaggggggcaagggccttatggtcccggagcgagcgcagcggcggcagctgccggaggttacggaccgggaagtggtcaacaaggtcccggacagcaaggtcccggccagcagggaccgggtcaacaaggtccaggcggacagggaccgtatgggcctggcgcatcagcagctgccgctgcggctggcggatatggccctgggagtggccaacaagggcctggccaacaaggtccgggtcagcagggaccaggccagcaaggtcgtggtgatagtccgggtccaggtcaacaggggccaggtcaacagggcccgggccaacaaggaccaggacaacaggggcctggacagcaaggccctggcggtcagggtgcctatggtccaggtgcttctgcagcagccggtgcagcgggcggttacggacccggtagcggccaacagggtcctggtcaacagggtccaggacagcagggtccgggtcaacagggacgtggtgattcaccgggtcctgggcagcaagggccggggcagcaaggtccgggacaacaaggacctggtcagcagggtccatacggtccaggtgccagcgcagctgcagcggccgcgggtggatacgggccaggctcaggccagcagggccctgggcagcagggccctggacagcaaggaccaggtgggcagggaccgtacggacccggtgctgcatcagcagcagttagcgttggtggctatggccctcagagcagcagcgttccggttgccagtgcagttgcaagccgtctgagcagtccggcagcaagcagtcgtgtgagcagcgcagtgagcagcctggttagctcaggtccgaccaaacatgcagcactgagcaataccattagcagcgttgttagccaggttagcgcaagcaatccgggtctgagcggttgtgatgttctggtgcaggcactgctggaagttgttagcgcactggttagcattctgggtagcagcagcattggtcagattaactatggtgcgagcgcacagtatacccagatggttggtcagagcgttgcacaggcactggcataa

SEQ ID NO: 44 - protein, 664 aa, Araneus diadematus
MHHHHHHHHHHSSGSSARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGRGDSPGPGQQGPGQQGPGQQGPGQQGPGQQGPGGQGAYGPGASAAAGAAGGYGPGSGQQGPGQQGPGQQGPGQQGRGDSPGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 45 - DNA, 2379 bp, Araneus diadematus
atgcatcatcaccaccatcaccatcatcatcacagcagcggtagcagcgcacgtgcaggtagcggtcagcagggtccgggtcagcaaggccctggtcaacagggacctggccagcagggaccgtatggtccgggtgcaagtgcagcagcagccgcagccggtggttatggtcctggttcaggccagcaaggtccgagccaacagggtccaggacaacaaggaccgggtggtcagggtccttatggccctggtgcatcagctgcggcagcggcagcaggcggatatggaccgggtagtggtcaggttaccggtcgtggtgatagtccggcaagcgtgacaggtcgcggtgattcaccggcaagccagggtcctggtggccaaggtccgtacggtcctggtagtagcgctgctgccgcagcagcgggtggtaacggtccgggtagcggacaacagggagccggtcaacaagggcccgggcaacaaggtcctggcgcaagcgcagccgctgctgctgcgggtggctatggcccaggttctggacagcaggttacaggccgtggcgattctcctgccagtgtgacgggacgtggcgatagccctgcatcaggccccgggcagcagggcccaggtggacaaggaccttatggaccaggtgcctccgcagccgcggcagccgccggtggatacggtccaggctctggtcaggtaacgggaagaggtgatagcccagcctcagttacgggtagaggcgattcaccagcatcaggaccgggacagcaaggacccggaggccagggaccatacggacctggtgcttcggctgccgctgccgccgctggcggttacggaccaggttccggccaacaaggcccaggacagcaggggcctggacaacagggtcccggaggacagggtccctatgggccaggcgctagtgcggctgcagctgctgcaggaggctacggaccgggatatggccaacaggtgactgggcgtggtgactctccggcatctgtaactggtcgtggcgacagtcctgccagcggaccaggccaacagggccctggaggtcaaggcccatacggcccaggcgcatcagcagccagcgcagcaagtggcgggtatggtccaggcagcggtcaacaggtcaccggtcggggagacagccctgcgtcagtaaccggacgcggagattctccagcaagcggaccggggcagcagggtcctggcgggcagggcccttacggacccggcgccagtgctgcggcggctgcggctggagggtatggacctggcagtggacagcagggtccaggccagcaggggcccggccagcaaggaccagggcaacaagggccaggcggacaggggccatacggtccgggagcttcagcagcggctgctgcggcagggggttacggccctggaagcggtcaacaaggtccgggacaacagggaccaggtcagcagggcccaggtcagcaggttacaggtcgcggagatagccctgccagcgtgaccggtagaggcgacagtccggcaagtggtcctggacagcaagggccaggacagcaaggtcctgggcagcagggaccgggtcaacagggtccaggccaacagggtcctggcggacagggtgcgtatggtcctggagcttctgccgcagcgggtgccgcaggcggttatggccccggatctggtcagcaaggacccggtcagcaaggaccgggacagcagggcccagggcaacaggtaactggcagaggcgatagtcctgcgtctgttactggacggggtgactcaccagcgagcgggcctggtcagcagggacctggacaacaagggcctggtcaacaaggcccaggccaacaagggccttatggtccaggcgctagcgcagcagcggcagctgctggcggttatggtccgggaagtggccaacaaggacccggccaacagggtccgggccagcaaggtccaggtggccaaggcccctatggccctggggcagcaagcgcagcagttagcgttggtggctatggtccgcagagcagcagcgttccggttgcgagcgcagttgcaagccgtctgtcaagtccggcagcaagtagccgtgtgagcagcgcagttagcagcctggttagcagcggtccgaccaaacatgcagcactgagcaataccattagcagcgttgttagccaggttagcgcaagcaatccgggtctgagcggttgtgatgttctggtgcaggcactgctggaagttgttagcgcactggtgagcattctgggtagcagcagcattggtcagattaactatggtgccagcgcacagtatacccagatggttggtcagagcgttgcacaggcactggcataa

SEQ ID NO: 46 - protein, 792 aa, Araneus diadematus
MHHHHHHHHHHSSGSSARAGSGQQGPGQQGPGQQGPGQQGPYGPGASAAAAAAGGYGPGSGQQGPSQQGPGQQGPGGQGPYGPGASAAAAAAGGYGPGSGQVTGRGDSPASVTGRGDSPASQGPGGQGPYGPGSSAAAAAAGGNGPGSGQQGAGQQGPGQQGPGASAAAAAAGGYGPGSGQQVTGRGDSPASVTGRGDSPASGPGQQGPGGQGPYGPGASAAAAAAGGYGPGSGQVTGRGDSPASVTGRGDSPASGPGQQGPGGQGPYGPGASAAAAAAGGYGPGSGQQGPGQQGPGQQGPGGQGPYGPGASAAAAAAGGYGPGYGQQVTGRGDSPASVTGRGDSPASGPGQQGPGGQGPYGPGASAASAASGGYGPGSGQQVTGRGDSPASVTGRGDSPASGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQVTGRGDSPASVTGRGDSPASGPGQQGPGQQGPGQQGPGQQGPGQQGPGGQGAYGPGASAAAGAAGGYGPGSGQQGPGQQGPGQQGPGQQVTGRGDSPASVTGRGDSPASGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 47 - DNA, 2316 bp, Araneus diadematus
atgcatcatcaccaccatcaccatcatcatcacagcagcggtagcagcgcacgtgcaggtagcggtcagcagggtccgggtcagcaaggccctggtcaacagggacctggccagcagggaccgtatggtccgggtgcaagtgcagcagcagccgcagccggtggttatggtcctggtagtggacagcaggttaccggtcgtggtgatagtccggcaagcggtccgagccagcaaggtccaggccaacaagggccaggtggtcagccgtatggccctggtgcttctgccgctgctgcagcagcgggtggctatggaccgggttcaggtcaggtgacaggtcgcggtgattcaccggcaagccagggtcctggtggtcagggtccttatggtccaggttcttcagcagctgcagcggcagcaggcggtaacggaccgggaagtggccagcaagtgacaggccgtggcgatagccctgccagcggtgctggacaacagggcccagggcaacaggggccgggagcatctgcagctgcggctgctgctggcggttatggcccaggctctggccaacaggttacgggacgtggcgattctcctgcatcaggccctggacagcaagggcccggaggacagggtccgtacggtcctggcgcatctgcggcagctgccgccgcaggcggatatggacctggcagcggtcaagtgactggacgcggagatagtccagcatcaggacccggtcaacaaggtcctggaggccaaggcccttacggacctggtgcaagcgctgcggccgcagcggctggaggttacggtccaggatcgggacaacaagtgaccggtagaggtgatagcccagcctcaggcccaggccagcagggtccaggacagcagggaccgggtggtcaaggtccatacggaccaggcgcaagtgccgccgcggctgccgcaggaggctacggccctggatatgggcagcaggtaacaggacggggtgacagccctgcgtcaggaccggggcagcaaggaccaggtggccagggaccctatgggccaggcgcatcagcagccagcgcagcaagtggtggatacggtcccggatcaggtcagcaagttacgggtagaggcgattcaccagccagcggaccaggacaacagggtcccggcggacaaggaccgtatggacccggagcatcagccgcagctgctgctgccggtggctacggtccaggttccggtcaacaggtaactggcagaggcgatagtcccgctagcgggcctgggcaacaaggtccggggcagcaggggccagggcagcaagggccaggcggacaaggcccttatggcccaggtgcctctgcagcagcggcagctgcgggaggatatggtccgggaagcggtcagcaaggacccggacagcaaggtccgggacaacagggtcctggtcagcaggttactggtcggggagactcaccggcatcagggccaggacaacaaggaccaggccagcaaggccctggccagcaaggaccgggtcaacaaggcccaggacaacaaggcccgggtggccagggtgcatacggtccgggtgcttcagctgcagccggtgcagcgggaggttatgggcctggctcaggtcagcaggggcccggccaacaaggtcccgggcaacaaggacctggacaacaggtgactgggcgtggtgactcaccagcgagtgggcctggacagcagggtcctggccaacagggcccaggtcagcaagggcctgggcagcagggcccttacggtccaggggcttctgctgccgcagctgcggcaggtggttatggaccaggaagtggtcaacaagtaaccggacggggagattctccagcgtctgggccaggtcagcagggaccaggtcaacagggtccaggtggccaggggccgtacggaccaggtgcagcaagcgcagcagttagcgttggtggctatggtccgcagagcagcagcgttccggttgcgagcgcagttgcaagccgtctgagcagtccggcagcaagcagtcgtgtgagcagcgcagttagcagcctggttagcagtggtccgaccaaacatgcagcactgagcaataccattagcagcgttgttagccaggttagcgcaagcaatccgggtctgagcggttgtgatgttctggttcaggcactgctggaagttgttagcgcactggtgagcattctgggtagcagcagcattggtcagattaactatggtgccagcgcacagtatacccagatggttggtcagagcgttgcacaggcactggcataa

SEQ ID NO: 48 - protein, 771 aa, Araneus diadematus
MHHHHHHHHHHSSGSSARAGSGQQGPGQQGPGQQGPGQQGPYGPGASAAAAAAGGYGPGSGQQVTGRGDSPASGPSQQGPGQQGPGGQPYGPGASAAAAAAGGYGPGSGQVTGRGDSPASQGPGGQGPYGPGSSAAAAAAGGNGPGSGQQVTGRGDSPASGAGQQGPGQQGPGASAAAAAAGGYGPGSGQQVTGRGDSPASGPGQQGPGGQGPYGPGASAAAAAAGGYGPGSGQVTGRGDSPASGPGQQGPGGQGPYGPGASAAAAAAGGYGPGSGQQVTGRGDSPASGPGQQGPGQQGPGGQGPYGPGASAAAAAAGGYGPGYGQQVTGRGDSPASGPGQQGPGGQGPYGPGASAASAASGGYGPGSGQQVTGRGDSPASGPGQQGPGGQGPYGPGASAAAAAAGGYGPGSGQQVTGRGDSPASGPGQQGPGQQGPGQQGPGGQGPYGPGASAAAAAAGGYGPGSGQQGPGQQGPGQQGPGQQVTGRGDSPASGPGQQGPGQQGPGQQGPGQQGPGQQGPGGQGAYGPGASAAAGAAGGYGPGSGQQGPGQQGPGQQGPGQQVTGRGDSPASGPGQQGPGQQGPGQQGPGQQGPYGPGASAAAAAAGGYGPGSGQQVTGRGDSPASGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 49 - DNA, 2256 bp, Araneus diadematus
atgcatcatcaccaccatcaccatcatcatcacagcagcggtagcagcctggaagttctgtttcagggtccggcacgtgcaggtagcggtcagcagggtcctggccagcaaggtccgggtcaacagggaccgggacagcagggaccttatggtccgggtgcaagcgcagcagcagccgcagccggtggttatggtcctggttcaggccaacagggtccaagtcagcaaggcccaggacaacaaggtcctggtggccagggtccgtatggccctggtgcatctgctgcggcagcggcagcaggcggatatggaccgggtagtggtcagggtaaaaaacagcgttttcgtcatcgtaatcgtaaaggtcagggtccaggtggtcaaggtccatacggtccgggtagcagtgcagctgctgcagccgctggcggtaatggtccaggtagtggacagcaaggtgcaggtcagcaaggacctgggcaacagggtccaggcgcaagtgcagccgcagctgctgcaggcggttatggtccaggctctggtcagcagggcaaaaaacaacgctttcgccaccgcaaccgcaaaggtggcccagggcagcagggtccgggtggacaaggcccttatggcccaggcgcatccgcagcagcggcagccgcagggggttacggacctggtagcggacaaggcaaaaaacagcggttccggcatcggaaccgcaaaggcggtccgggacagcaaggccctggcggtcaaggtccttatggtcctggggcatcagctgcagccgcagcggcagggggatatggccctggaagtggccagcagggaccaggccaacaagggccagggcagcaagggccaggtggtcagggaccttacggaccaggtgcctcagcggcagcagccgcagctgggggatacggtccgggttatggtcagcaaggtaaaaaacagcgcttccgtcaccgcaatcggaaaggcggacctggtcaacaaggaccaggtggacaagggccttacggtcctggcgctagtgcagcaagcgcagccagcggtggctatggtccgggaagcggacaacagggaaaaaaacaacgtttccggcaccggaatcgtaaaggtgggcctggacaacagggacctggcggtcagggtccttacggtccgggtgcttctgcagcggcagccgcagcgggtggctatggcccaggtagtggtcaacaaggccctggtcagcagggacctggacaacaaggccctggacaacaagggccagggggtcaaggcccttatggaccgggtgccagtgccgcagctgcagcggcaggcggttacggtccgggttccggacaacaaggaccgggtcagcaaggtccaggccagcagggtccagggcagcagggaaaaaaacagcggttccgccaccgtaatcgcaaagggggtccaggccaacaaggacctggccaacaaggtcctggtcagcaaggccctgggcagcaaggcccaggacagcagggaccaggtgggcagggtgcgtatgggccaggggctagtgctgcagcgggtgctgcgggtggatacggaccgggatctggacaacagggtcctggtcaacagggtccaggacagcaaggtcctggacagcaagggaaaaaacaacggtttcgtcatcgtaaccggaaagggggtcctggccagcagggaccgggacagcaaggaccaggtcaacaaggcccaggtcaacaaggaccttatggtccaggtgcgtcagcagccgcagctgcagcgggtggatacggtcctggtagcggtcagcaagggccaggtcaacagggacctggtcagcagggtccgggtggtcagggtccttatggacctggcgcagcaagtgcagcagttagcgttggtggttatggaccgcagagcagcagcgttccggttgccagcgcagttgcaagccgtctgagcagtccggcagcaagcagtcgtgttagcagcgcagtgagcagtctggttagctcaggtccgaccaaacatgcagcactgagcaataccattagcagcgttgttagccaggttagcgcaagcaatccgggtctgagcggttgtgatgttctggtgcaggcactgctggaagttgttagcgcactggttagcattctgggtagcagcagcattggtcagattaactatggtgcgagcgcacagtatacccagatggttggtcagagcgttgcacaggcactggcctaa

SEQ ID NO: 50 - protein, 751 aa, Araneus diadematus
MHHHHHHHHHHSSGSSLEVLFQGPARAGSGQQGPGQQGPGQQGPGQQGPYGPGASAAAAAAGGYGPGSGQQGPSQQGPGQQGPGGQGPYGPGASAAAAAAGGYGPGSGQGKKQRFRHRNRKGQGPGGQGPYGPGSSAAAAAAGGNGPGSGQQGAGQQGPGQQGPGASAAAAAAGGYGPGSGQQGKKQRFRHRNRKGGPGQQGPGGQGPYGPGASAAAAAAGGYGPGSGQGKKQRFRHRNRKGGPGQQGPGGQGPYGPGASAAAAAAGGYGPGSGQQGPGQQGPGQQGPGGQGPYGPGASAAAAAAGGYGPGYGQQGKKQRFRHRNRKGGPGQQGPGGQGPYGPGASAASAASGGYGPGSGQQGKKQRFRHRNRKGGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQGKKQRFRHRNRKGGPGQQGPGQQGPGQQGPGQQGPGQQGPGGQGAYGPGASAAAGAAGGYGPGSGQQGPGQQGPGQQGPGQQGKKQRFRHRNRKGGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 51 - DNA, 2025 bp, Araneus diadematus
atgcatcatcaccaccatcaccatcatcatcacagcagcggtagcagcgcacgtgcaggtagcggtcagcagggtccgggtcagcaaggccctggtcaacagggacctggccagcagggaccgtatggtccgggtgcaagtgcagcagcagccgcagccggtggttatggtcctggttcaggccagcaaggtccgagccaacagggtccaggacaacaaggaccgggtggtcagggtccttatggccctggtgcatcagctgcggcagcagcggcagttaccggtcgtggtgatagtccggcaagccagggtcctggtggccaaggtccgtacggtcctggtagttctgctgccgctgccgcagcaggcggtaacggtccaggcagtggacagcagggtgctgggcaacagggcccaggtcagcaaggtccaggtgcatcagcagcagcggctgcagccgtgaccggtcgcggagattcaccggcaagcggaccggggcaacaaggtcctggcggacaaggcccttatggtccaggagcgagcgctgctgctgcagctgcggtgacaggtcgtggcgactctccggcatcaggacccggccaacaagggcccggaggtcaaggaccttacggaccaggtgccagtgccgctgcagcggcggcaggcggttatggaccgggaagcgggcagcaagggccagggcagcaggggcctggacaacagggtcccggtggacagggcccatacggacctggtgcaagcgcagccgccgcagcagcagtaacaggccgtggcgatagccctgcaagcggtcctggacagcaagggcctggagggcagggtccatacggtccgggagcatcagccgcatctgcagcaagcgttacaggacgcggtgattctccagccagcgggccaggtcaacaaggcccaggcggacagggtccgtacggcccaggcgctagtgcagccgctgctgccgcaggaggatacggaccgggtagtggtcagcaaggacccggacagcaaggtccgggacagcagggaccgggccaacaaggaccagggggccagggaccctacgggccaggagcttccgcggctgccgcggcagccggaggctatggcccaggtagtggccaacaaggtccaggccagcaaggccccgggcaacaagggccgggtcaacaggttacaggccgtggtgacagtcctgcctcaggtcctgggcagcagggtcccggccagcaggggccaggacagcaaggtcctggccaacaaggcccaggacagcagggtccgggtggccagggtgcttatggtccaggcgcttctgcagcagccggtgctgcgggtggttacggtcccggtagcggacaacaggggccaggccaacagggacccgggcagcaaggacctggccagcaagtgaccggtcgcggtgatagccctgcatcaggtccagggcaacagggacctggtcaacaagggcctgggcaacaaggaccaggccaacaaggtccgtatggtccaggtgcctccgcagctgcagcagctgctggcggatacggtccaggatcaggtcagcagggaccagggcaacagggtccggggcagcagggtcctggtggtcagggaccgtatggacctggcgcagcaagcgcagcagttagcgttggtggctatggaccgcagagcagcagtgttccggttgccagcgcagttgcaagccgtctgagcagtccggcagcaagcagtcgtgttagcagtgcagttagcagcctggttagcagcggtccgaccaaacatgcagcactgagcaataccattagcagcgttgttagccaggttagcgcaagcaatccgggtctgagcggttgtgatgttctggtgcaggcactgctggaagttgttagcgcactggtgagcattctgggtagcagcagcattggtcagattaactatggtgcgagcgcacagtatacccagatggttggtcagagcgttgcacaggcactggcataa

SEQ ID NO: 52 - protein, 674 aa, Araneus diadematus
MHHHHHHHHHHSSGSSARAGSGQQGPGQQGPGQQGPGQQGPYGPGASAAAAAAGGYGPGSGQQGPSQQGPGQQGPGGQGPYGPGASAAAAAAVTGRGDSPASQGPGGQGPYGPGSSAAAAAAGGNGPGSGQQGAGQQGPGQQGPGASAAAAAAVTGRGDSPASGPGQQGPGGQGPYGPGASAAAAAAVTGRGDSPASGPGQQGPGGQGPYGPGASAAAAAAGGYGPGSGQQGPGQQGPGQQGPGGQGPYGPGASAAAAAAVTGRGDSPASGPGQQGPGGQGPYGPGASAASAASVTGRGDSPASGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQVTGRGDSPASGPGQQGPGQQGPGQQGPGQQGPGQQGPGGQGAYGPGASAAAGAAGGYGPGSGQQGPGQQGPGQQGPGQQVTGRGDSPASGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

SEQ ID NO: 53 - DNA, 2019 bp, Araneus diadematus
atgcatcatcaccaccatcaccatcatcatcacagcagcggtagcagcgcacgtgcaggtagcggtcagcagggtccgggtcagcaaggccctggtcaacagggacctggccagcagggaccgtatggtccgggtgcaagtgcagcagcagccgcagccggtggttatggtcctggttcaggccagcaaggtccgagccaacagggtccaggacaacaaggaccgggtggtcagggtccttatggccctggtgcatcagctgcggcagcggcagcaggcggatatggaccgggttctgggcaacaaggtcctggcggtcaaggcccttacggtccgggtagtagcgctgctgccgcagcagcgggtggtaatggcccaggcagtggacagcagggtgctggacaacagggcccaggtcagcaaggtccaggtgcatcagccgctgctgctgcagccggtggctacggaccaggttctggtcagcaaggaccgggacagcaggggccaggtggccaaggtccgtatggaccaggagccagtgctgctgcggctgccgctggcggttacggtccaggatcaggtcaaggacccggccaacaaggccccggtggacaaggaccatacggtcctggtgcctccgcagccgcggctgcagcgggaggatatgggcctggaagcgggcagcagggacccggacaacagggtcccgggcaacaggggcctggcggacagggtccctacggacctggcgctagtgcagcggctgcggccgctggaggctatggacctggctacggtcaacaagggccagggcagcagggtcctggaggccagggaccttacggcccaggcgcaagcgctgcaagcgcagcatcaggtggttacggccctgggagcggacagcaaggcccagggcaacaaggcccaggggggcaagggccttatggtcccggagcgagcgcagcggcggcagctgccggaggttacggaccgggaagtggtcaacaaggtcccggacagcaaggtcccggccagcagggaccgggtcaacaaggtccaggcggacagggaccgtatgggcctggcgcatcagcagctgccgctgcggctggcggatatggccctgggagtggccaacaagggcctggccaacaaggtccgggtcagcagggaccagggcagcaggttaccggtcgtggtgatagtccggcaagcgggccaggccaacagggccctggacagcaggggcctggacagcaaggtccgggacaacaagggcctggtcagcaagggcctggtggtcagggtgcctacggtccgggtgcttcagcagctgcaggggctgccggtggatatggcccaggctcaggtcaacagggtcccggtcagcagggtccgggacaacagggaccaggccaacaagtgacaggtcgcggtgattcaccggcatctggcccaggacagcaaggacctggtcagcagggtccaggccaacaaggtcctggccagcagggtccttatggacccggagcctcagcagctgcagcagccgcaggcggctatggtcctggtagtgggcagcaaggaccaggccagcaaggaccaggacaacagggaccgggaggccaaggaccgtacggacccggtgcagcaagcgcggcagttagcgttggtggatacggtccgcagagcagcagcgttccggttgccagcgcagttgcaagccgtctgagcagtccggcagcaagcagtcgtgttagcagtgcagttagcagcctggttagctcaggtccgaccaaacatgcagcactgagcaataccattagcagcgttgtgagccaggttagcgcaagcaatccgggtctgagcggttgtgatgttctggtgcaggcactgctggaagttgttagcgcactggtgagcattctgggtagcagcagcattggtcagattaactatggtgcgagcgcacagtatacccagatggttggtcagagcgttgcacaggcactggcataa

SEQ ID NO: 54 - protein, 672 aa, Araneus diadematus
MHHHHHHHHHHSSGSSARAGSGQQGPGQQGPGQQGPGQQGPYGPGASAAAAAAGGYGPGSGQQGPSQQGPGQQGPGGQGPYGPGASAAAAAAGGYGPGSGQQGPGGQGPYGPGSSAAAAAAGGNGPGSGQQGAGQQGPGQQGPGASAAAAAAGGYGPGSGQQGPGQQGPGGQGPYGPGASAAAAAAGGYGPGSGQGPGQQGPGGQGPYGPGASAAAAAAGGYGPGSGQQGPGQQGPGQQGPGGQGPYGPGASAAAAAAGGYGPGYGQQGPGQQGPGGQGPYGPGASAASAASGGYGPGSGQQGPGQQGPGGQGPYGPGASAAAAAAGGYGPGSGQQGPGQQGPGQQGPGQQGPGGQGPYGPGASAAAAAAGGYGPGSGQQGPGQQGPGQQGPGQQVTGRGDSPASGPGQQGPGQQGPGQQGPGQQGPGQQGPGGQGAYGPGASAAAGAAGGYGPGSGQQGPGQQGPGQQGPGQQVTGRGDSPASGPGQQGPGQQGPGQQGPGQQGPYGPGASAAAAAAGGYGPGSGQQGPGQQGPGQQGPGGQGPYGPGAASAAVSVGGYGPQSSSVPVASAVASRLSSPAASSRVSSAVSSLVSSGPTKHAALSNTISSVVSQVSASNPGLSGCDVLVQALLEVVSALVSILGSSSIGQINYGASAQYTQMVGQSVAQALA

(19) United States
(12) Patent Application Publication
(10) Pub. No.: US 2019/0016763 A1
(43) Pub. Date: Jan. 17, 2019

(54) Fibroin-Like Protein Variant and Cell Culture Method

(71) Applicant: AJINOMOTO CO., INC., Tokyo (JP)

(72) Inventors: Manabu Kitazawa, Kanagawa (JP); Nao Sugimoto, Kanagawa (JP); Riho Kodama, Kanagawa (JP); Takeshi Nagahiko, Kanagawa (JP); Yoshihiro Ito, Kanagawa (JP); Kentaro Nakase, Kanagawa (JP); Yoshinori Takashima, Kanagawa (JP); Ayako Mine, Kanagawa (JP); Yoko Kuwabara, Kanagawa (JP); Uno Tagami, Kanagawa (JP); Masayuki Sugiki, Kanagawa (JP)

(73) Assignee: AJINOMOTO CO., INC., Tokyo (JP)

(21) Appl. No.: 16/144,562

(22) Filed: Sep. 27, 2018

Related U.S. Application Data
(63) Continuation of application No. PCT/JP2017/013587, filed on Mar. 31, 2017.

(30) Foreign Application Priority Data
Mar. 31, 2016 (JP) 2016-071530
Oct. 21, 2016 (JP) 2016-207359

(51) Int. Cl. C07K 14/435; C12N 15/62; C12N 5/00; C07K 2/00; C12M 3/04; C12N 5/071; C12N 5/074

(52) U.S. Cl. CPC C07K 14/43518; C12N 15/62; C12N 5/0068; C07K 2/00; C12M 3/04; C12N 2533/50; C12N 5/0607; C12N 5/0696; C07K 2319/70; C12N 2539/00; C12N 5/0692

(57) ABSTRACT

A technique for efficiently culturing cells such as stem cells is provided. Cells such as stem cells are cultured using a cell culture vessel coated with a fibroin-like protein into which a cell adhesion sequence containing RGD (Arg-Gly-Asp) is inserted.

This application is a Continuation of, and claims priority under 35 U.S.C. § 120 to, International Application No. PCT/JP2017/013587, filed Mar. 31, 2017, and claims priority therethrough under 35 U.S.C. § 119 to Japanese Patent Application Nos. 2016-071530 and 2016-207359, filed Mar. 31, 2016 and Oct. 21, 2016, respectively, the entireties of which are incorporated by reference herein. Also, the Sequence Listing filed electronically herewith is hereby incorporated by reference (File name: 2018-09-27T US-586 Seq List; File Size: 133 KB; Date recorded: Sep. 27, 2018).

### BACKGROUND OF THE INVENTION

### Field of the Invention

The present invention relates to a fibroin-like protein variant and a cell culture method using the same.

### Brief Description of the Related Art

Stem cells, particularly pluripotent stem cells such as embryonic stem cells (ES cells) and induced pluripotent stem cells (iPS cells), are expected to be applied to regenerative medicine. To culture stem cells, feeder cells are usually used. However, due to constraints in clinical applications and the like, technology for replacing feeder cells has been developed.

For example, it is known that cell adhesion proteins, such as fibronectin or laminin, can be used as a scaffold to cultivate stem cells. It is also known that a fibroin variant obtained by introducing a cell adhesion sequence into spider or silk fibroin can be used as a scaffold for cell culture when cultivating stem cells (see WO2015/036619, JP2013-523174, and Bini et al., Biomacromolecules (2006) 7(11):3139-45). An amino acid sequence containing RGD (Arg-Gly-Asp) is know to be a typical example of the cell adhesion sequence.

However, it has not been reported whether stem cells can be efficiently cultured by using a fibroin variant having a fibroin repetitive structure introduced with a cell adhesion sequence containing RGD as a scaffold for cell culture.

### SUMMARY OF THE INVENTION

A technique for efficiently culturing cells such as stem cells is described herein and provided by the present invention.

It is described herein that stem cells can be efficiently cultured by using a fibroin-like protein variant having a fibroin-like protein repetitive structure containing a cell adhesion sequence including RGD as a scaffold for cell culture.

It is an aspect of the present invention to provide a protein comprising a repetitive structure and a cell adhesion sequence, wherein the repetitive structure consists of a repeating unit repeats 3 to 15 times, wherein each repeating unit consists of an Ala-rich region and an Ala-non-rich region mutually linked via a linkage site, wherein each Ala-rich region consists of an amino acid sequence having a length of 8 to 54 residues, wherein the ratio of Ala residues in each Ala-rich region is 30% or more, wherein each Ala-rich region contains at least one Ala residue for every four consecutive amino acid residues, wherein each Ala-non-rich region consists of an amino acid sequence having a length of 4 residues or longer, wherein the ratio of Ala residues in each Ala-non-rich region is 20% or less, wherein one or more of said repeating units in the repetitive structure comprises the cell adhesion sequence, and wherein each cell adhesion sequence consists of an amino acid sequence comprising Arg-Gly-Asp and has a length of 3 to 18 residues.

It is a further aspect of the present invention to provide the protein as described above, wherein said cell adhesion sequence is present in two or more repeating units in the repetitive structure.

It is a further aspect of the present invention to provide the protein as described above, wherein the total number of said cell adhesion sequence that is present in the protein is 1 to 50.

It is a further aspect of the present invention to provide the protein as described above, wherein each cell adhesion sequence is present in the Ala-non-rich region, or in the linkage site between the Ala-rich region and the Ala-non-rich region

It is a further aspect of the present invention to provide the protein as described above, wherein each cell adhesion sequence consists of an amino acid sequence selected from the group consisting of: (a) the amino acid sequence of SEQ ID NO: 22, 23, 24, 25, 30, or 31; (b) the amino acid sequence of SEQ ID NO: 22, 23, 24, 25, 30, or 31, but wherein said sequence includes substitution, deletion, insertion, and/or addition of 1 to 3 amino acid residues at position(s) other than Arg-Gly-Asp; and (c) an amino acid sequence having an identity of 80% or higher to the amino acid sequence of SEQ ID NO: 22, 23, 24, 25, 30, or 31, provided that Arg-Gly-Asp is conserved.

It is a further aspect of the present invention to provide the protein as described above, wherein each Ala-rich region consists of an amino acid sequence having a length of 8 to 15 residues.

It is a further aspect of the present invention to provide the protein as described above, wherein each Ala-rich region consists of an amino acid sequence having a length of 9 residues.

It is a further aspect of the present invention to provide the protein as described above, wherein each Ala-rich region contains one or more Gly residues and one or more Ser residues.

It is a further aspect of the present invention to provide the protein as described above, wherein the ratio of Ala residue in each Ala-rich region is 50% or more.

It is a further aspect of the present invention to provide the protein as described above, wherein the ratio of Ala, Gly, and Ser residues in each Ala-rich region is 90% or more in total.

It is a further aspect of the present invention to provide the protein as described above, wherein each Ala-rich region consists of an amino acid sequence selected from the group consisting of: (a) the amino acid sequence of SEQ ID NO: 7, 8, 9, or 10; (b) the amino acid sequence of SEQ ID NO: 7, 8, 9, or 10, but wherein said sequence includes substitution, deletion, insertion, and/or addition of 1 to 3 amino acid residues; and (c) an amino acid sequence having an identity of 80% or higher to the amino acid sequence of SEQ ID NO: 7, 8, 9, or 10.

It is a further aspect of the present invention to provide the protein as described above, wherein each Ala-non-rich region consists of an amino acid sequence having a length of 15 to 100 residues.

It is a further aspect of the present invention to provide the protein as described above, wherein the ratio of Ala residue in each Ala-non-rich region is 5% or less.

It is a further aspect of the present invention to provide the protein as described above, wherein the ratio of Gly, Ser, Gln, Pro, and Tyr residues in each Ala-non-rich region is 90% or more in total.

It is a further aspect of the present invention to provide the protein as described above, wherein each Ala-non-rich region comprises an amino acid sequence selected from the group consisting of: (a) one or more of the amino acid sequences of SEQ ID NOS: 11 to 21; (b) one or more of the amino acid sequences of SEQ ID NOS: 11 to 21, but wherein said sequence includes substitution, deletion, insertion, and/or addition of 1 to 3 amino acid residues; and (c) one or more amino acid sequences having an identity of 80% or higher to the amino acid sequences of SEQ ID NOS:11 to 21.

It is a further aspect of the present invention to provide the protein as described above, wherein each Ala-non-rich region comprises an amino acid sequence selected from the group consisting of: (a) the amino acid sequence of amino acid numbers of 62 to 91, 101 to 120, 130 to 151, 161 to 185, 195 to 218, 228 to 257, 267 to 291, 301 to 325, 335 to 369, 379 to 438, 448 to 497, or 507 to 536 of SEQ ID NO: 2, or a partial sequence thereof; (b) the amino acid sequence of amino acid numbers of 62 to 91, 101 to 120, 130 to 151, 161 to 185, 195 to 218, 228 to 257, 267 to 291, 301 to 325, 335 to 369, 379 to 438, 448 to 497, or 507 to 536 of SEQ ID NO: 2, or a partial sequence thereof, but wherein said sequence includes substitution, deletion, insertion, and/or addition of 1 to 10 amino acid residues; and (c) an amino acid sequence showing an identity of 90% or higher to the amino acid sequence of amino acid numbers of 62 to 91, 101 to 120, 130 to 151, 161 to 185, 195 to 218, 228 to 257, 267 to 291, 301 to 325, 335 to 369, 379 to 438, 448 to 497, or 507 to 536 of SEQ ID NO: 2, or a partial sequence thereof.

It is an aspect of the present invention to provide a composition containing the protein as described above.

It is a further aspect of the present invention to provide the composition as described above, wherein the composition is a cell scaffold material.

It is a further aspect of the present invention to provide the composition as described above, wherein the cell scaffold material is for culturing a stem cell.

It is a further aspect of the present invention to provide the composition as described above, wherein the stem cell is an embryonic stem cell or an induced pluripotent stem cell.

It is a further aspect of the present invention to provide the composition as described above, wherein the composition is a coating agent for a cell culture vessel.

It is a further aspect of the present invention to provide the composition as described above, wherein the cell culture vessel is for culturing a stem cell.

It is a further aspect of the present invention to provide the composition as described above, wherein the stem cell is an embryonic stem cell or an induced pluripotent stem cell.

It is a further aspect of the present invention to provide the composition as described above, wherein the composition further contains another protein, wherein said another protein is a protein other than the proteins as described above.

It is a further aspect of the present invention to provide the composition as described above, wherein said another protein is selected from the group consisting of: (1) a protein that stabilizes a component in a culture medium; (2) a protein comprising an amino acid sequence that stabilizes a component in a culture medium; (3) a protein having a cell adhesion ability; (4) a protein comprising an amino acid sequence having a cell adhesion ability; (5) a protein that promotes proliferation of a cell; (6) a protein comprising an amino acid sequence that promotes proliferation of a cell; (7) a protein that binds to any one of the proteins (1) to (6); and (8) a protein comprising an amino acid sequence that binds to any one of the proteins (1) to (6).

It is an aspect of the present invention to provide an apparatus comprising the protein as described above.

It is a further aspect of the present invention to provide the apparatus as described above, wherein the apparatus is an apparatus for medical or research use.

It is a further aspect of the present invention to provide the apparatus as described above, wherein the apparatus is a cell culture vessel.

It is a further aspect of the present invention to provide the apparatus as described above, wherein the apparatus is a cell scaffold material.

It is a further aspect of the present invention to provide the apparatus as described above, wherein a cell culture surface of the apparatus has been coated with the protein.

It is a further aspect of the present invention to provide the apparatus as described above, wherein the apparatus is for culturing a stem cell.

It is a further aspect of the present invention to provide the apparatus as described above, wherein the stem cell is an embryonic stem cell or an induced pluripotent stem cell.

It is a further aspect of the present invention to provide the apparatus as described above, wherein the apparatus further comprises another protein, wherein said another protein is a protein other than the proteins as described above.

It is a further aspect of the present invention to provide the apparatus as described above, wherein said another protein is selected from the group consisting of: (1) a protein that stabilizes a component in a culture medium; (2) a protein comprising an amino acid sequence that stabilizes a component in a culture medium; (3) a protein having a cell adhesion ability; (4) a protein comprising an amino acid sequence having a cell adhesion ability; (5) a protein that promotes proliferation of a cell; (6) a protein comprising an amino acid sequence that promotes proliferation of a cell; (7) a protein that binds to any one of the proteins (1) to (6); and (8) a protein comprising an amino acid sequence that binds to any one of the proteins (1) to (6).

It is an aspect of the present invention to provide a method for producing a cultured cell, the method comprising: culturing a cell by using the apparatus as described above.

It is a further aspect of the present invention to provide the method as described above, wherein the cell to be cultured is a stem cell.

It is a further aspect of the present invention to provide the method as described above, wherein the stem cell is an embryonic stem cell or an induced pluripotent stem cell.

It is a further aspect of the present invention to provide the method as described above, wherein another protein other than the proteins as described above, is further used in combination.

It is a further aspect of the present invention to provide the method as described above, wherein said another protein is selected from the group consisting of: (1) a protein that stabilizes a component in a culture medium; (2) a protein comprising an amino acid sequence that stabilizes a component in a culture medium; (3) a protein having a cell adhesion ability; (4) a protein comprising an amino acid sequence having a cell adhesion ability; (5) a protein that promotes proliferation of a cell; (6) a protein comprising an amino acid sequence that promotes proliferation of a cell; (7) a protein that binds to any one of the proteins (1) to (6); and (8) a protein comprising an amino acid sequence that binds to any one of the proteins (1) to (6).

It is an aspect of the present invention to provide a gene encoding the protein as described above.

It is an aspect of the present invention to provide a vector comprising the gene as described above.

It is an aspect of the present invention to provide a host comprising the gene as described above.

### BRIEF DESCRIPTION OF THE DRAWINGS

FIG. 1 is a graph showing results of evaluating a scaffold function for an iPS cell using various cell culture vessels coated with a cell adhesion sequence-inserted fibroin-like protein or with an existing basement membrane matrix or a mimic thereof.

FIG. 2 is a graph showing results of evaluating a scaffold function for an iPS cell using a cell culture plate immediately after coating with a cell adhesion sequence-inserted fibroin-like protein, or after storage for 6 days at 4° C. after the coating.

FIG. 3 is a graph showing results of evaluating a scaffold function for an iPS cell using various culture media with a cell culture plate coated with a cell adhesion sequence-inserted fibroin-like protein.

FIG. 4 is a graph showing results of evaluating a scaffold function for an iPS cell over a period of 3 weeks using a cell culture plate coated with a cell adhesion sequence-inserted fibroin-like protein. The horizontal axis represents “week”.

FIG. 5 is a graph showing results of evaluating a scaffold function for an iPS cell using a cell culture plate immediately after coating with a cell adhesion sequence-inserted fibroin-like protein, or after storage for 6 days at 37° C. after the coating.

FIG. 6 is a graph showing results of evaluating a scaffold function for an iPS cell over a period of 3 weeks using a cell culture plate coated with a cell adhesion sequence-inserted fibroin-like protein. The horizontal axis represents “week”.

FIG. 7 is a graph showing results of evaluating a scaffold function for an iPS cell using a cell culture plate coated with a cell adhesion sequence-inserted fibroin-like protein and/or a heparin-binding sequence-inserted fibroin-like protein.

### DETAILED DESCRIPTION OF EXEMPLARY EMBODIMENTS

<1> Protein of the Present Invention

<1-1> Protein

The protein as described herein is a protein that includes a repetitive structure and a cell adhesion sequence.

The protein as described herein may specifically be a fibroin-like protein into which a cell adhesion sequence has been inserted. Hence, the protein as described herein can also be referred to as “cell adhesion sequence-inserted fibroin-like protein”.

The term “fibroin-like protein” can be a generic term referring to fibroin or a protein having a structure similar to that of fibroin.

The term “fibroin” can refer to a fibrous protein that makes up a spider's thread or silkworm's thread. That is, examples of fibroin can include fibroin of spider, and fibroin of silkworm. Species of spider, species of silkworm, and type of thread are not particularly limited. Examples of spider can include Araneus diadematus and Nephila clavipes. Other examples of spider can include Araneus bicentenarius, Argiope amoena, Argiope aurantia, Argiope trifasciata, Cyrtophora moluccensis, Dolomedes tenebrosus, Euprosthenops australis, Gasteracantha mammosa, Latrodectus geometricus, Latrodectus hesperus, Macrothele holsti, Nephila pilipes, Nephila madagascariensis, Nephila senegalensis, Octonoba varians, Psechrus sinensis, Tetragnatha kauaiensis, and Tetragnatha versicolor. Examples of fibroin of spider include proteins that make up drag line, frame thread, and radius thread produced by major ampullate gland, such as major ampullate gland proteins, proteins of scaffolding thread produced by minor ampullate gland, such as minor ampullate gland proteins, and proteins of spiral line produced by flagelliform gland, such as flagelliform gland proteins. Specific examples of fibroin of spider can include, for example, the major ampullate gland proteins ADF3 and ADF4 of Araneus diadematus and the major ampullate gland proteins MaSp1 and MaSp2 of Nephila clavipes. Examples of silkworm can include Bombyx mori and Samia cynthia. The amino acid sequences of these fibroins and the nucleotide sequences of the genes encoding these fibroins (also referred to as “fibroin gene”) can be obtained from public databases such as NCBI (ncbi.nlm.nih.gov).

The term “protein having a structure similar to that of fibroin” can refer to a protein having a sequence similar to a repetitive sequence of fibroin. The “sequence similar to a repetitive sequence of fibroin” may be a sequence actually found in fibroin, or may be a sequence similar to such a sequence. Examples of the sequence similar to a repetitive sequence of fibroin can include the “repetitive sequence” as described herein. Examples of the protein having a structure similar to that of fibroin can include polypeptides derived from the large spigot drag line proteins described in WO2012/165476 and recombinant spider silk proteins described in WO2006/008163. Examples of the protein having a structure similar to that of fibroin also can include the ADF3 protein used in the Examples section. The nucleotide sequence of the ADF3 gene used in the Examples section is shown in SEQ ID NO: 1, and the amino acid sequence of the ADF3 protein encoded by the gene is shown in SEQ ID NO: 2. Incidentally, the nucleotide sequence of positions 7-1989 of SEQ ID NO: 1 encodes the amino acid sequence shown in SEQ ID NO: 2. The protein having a structure similar to that of fibroin may or may not be a fibrous protein.

The term “fibrous protein” can refer to a protein that has a fibrous form under predetermined conditions. That is, the fibrous protein may be a protein expressed in a fibrous form, or a protein that is not in a fibrous form when it is expressed, but can be processed into a fibrous form. The fibrous protein may be, for example, a protein that is expressed as an inclusion body, and can be then processed into a fibrous form by an appropriate technique. Examples of methods for processing a protein into a fibrous form include the method disclosed in WO2012/165476.

That is, the fibroin-like protein may be, for example, a protein having any of the amino acid sequences of the fibroin-like proteins disclosed in the aforementioned database or documents, or a protein having the amino acid sequence shown in SEQ ID NO: 2. The fibroin-like protein may also be, for example, a protein having a partial sequence of any of the aforementioned amino acid sequences. Examples of a partial sequence of an amino acid sequence can include a portion having a sequence similar to the repetitive sequence of fibroin. Specific examples of the partial sequence of an amino acid sequence can include, for example, positions 25-610 and positions 53-536 in the amino acid sequence shown in SEQ ID NO:. Similarly, a gene encoding a fibroin-like protein (also referred to as “fibroin-like protein gene”) may be, for example, a gene having any of the nucleotide sequences of the fibroin genes disclosed in the aforementioned database or documents, or a gene having the nucleotide sequence of positions 7-1989 of SEQ ID NO: 1. The fibroin-like protein gene may also be, for example, a gene having a partial sequence of any of the aforementioned nucleotide sequences. Examples of the partial sequence of a nucleotide sequence can include a portion encoding an amino acid sequence having a sequence similar to the repetitive sequence of fibroin. The expression “having an (amino acid or nucleotide) sequence” can encompass either a larger sequence that includes the amino acid or nucleotide sequence, or having only the amino acid or nucleotide sequence”.

The fibroin-like protein may also be a variant of any of the fibroin-like proteins exemplified above, that is, fibroin and the proteins having a structure similar to that of fibroin exemplified above, so long as the original function thereof is maintained. Similarly, the fibroin-like protein gene may also be a variant of any of the fibroin-like protein genes exemplified above, that is, genes encoding fibroin and the proteins having a structure similar to that of fibroin exemplified above, so long as the original function thereof is maintained. Such variants that maintain the original function can also be referred to as “conservative variants”. Examples of the conservative variants can include, for example, homologues and artificially-modified proteins or genes of the fibroin-like proteins exemplified above and genes encoding them.

The expression “the original function is maintained” can mean that a variant of a gene or protein has a function (e.g. activity and property) corresponding to the function (e.g. activity and property) of the original gene or protein. That is, in the case of the fibroin-like protein, the expression “the original function is maintained” can mean that a variant of the protein has a scaffold function for a cell, such as a stem cell, due to insertion of a cell adhesion sequence. In the case of the fibroin-like protein gene, the expression “the original function is maintained” can mean that a variant of the gene encodes a protein that maintains the original function, namely, a protein showing a scaffold function for a cell, such as a stem cell, due to insertion of a cell adhesion sequence.

Examples of homologues of the fibroin-like protein can include, for example, a protein obtained from a public database by BLAST search or FASTA search using any of the aforementioned amino acid sequences of fibroin-like proteins as a query sequence. Also, a homologue of the aforementioned fibroin-like protein genes can be obtained by, for example, PCR using a chromosome of various microorganisms as the template, and oligonucleotides prepared on the basis of any of the aforementioned nucleotide sequences of fibroin-like protein genes as the primers.

Conservative variants of the fibroin-like protein and fibroin-like protein gene will be explained below.

The fibroin-like protein may be a protein having any of the aforementioned amino acid sequences of fibroin-like proteins including substitution, deletion, insertion, and/or addition of one or several amino acid residues at one or several positions, so long as the original function of the protein is maintained. Although the number meant by the term “one or several” can differ depending on the positions of amino acid residues in the three-dimensional structure of the protein, or the types of amino acid residues, it is specifically, for example, 1 to 50, 1 to 40, 1 to 30, 1 to 20, 1 to 10, 1 to 5, or 1 to 3.

The aforementioned substitution, deletion, insertion, and/or addition of one or several amino acid residues are/is each a conservative mutation that maintains the original function of the protein. Typical examples of the conservative mutation are conservative substitutions. The conservative substitution is a mutation wherein substitution takes place mutually among Phe, Trp, and Tyr, if the substitution site is an aromatic amino acid; among Leu, Ile, and Val, if it is a hydrophobic amino acid; between Gln and Asn, if it is a polar amino acid; among Lys, Arg, and His, if it is a basic amino acid; between Asp and Glu, if it is an acidic amino acid; and between Ser and Thr, if it is an amino acid having a hydroxyl group. Examples of substitutions considered as conservative substitutions can include, specifically, substitution of Ser or Thr for Ala, substitution of Gln, His, or Lys for Arg, substitution of Glu, Gln, Lys, His, or Asp for Asn, substitution of Asn, Glu, or Gln for Asp, substitution of Ser or Ala for Cys, substitution of Asn, Glu, Lys, His, Asp, or Arg for Gln, substitution of Gly, Asn, Gln, Lys, or Asp for Glu, substitution of Pro for Gly, substitution of Asn, Lys, Gln, Arg, or Tyr for His, substitution of Leu, Met, Val, or Phe for Ile, substitution of Ile, Met, Val, or Phe for Leu, substitution of Asn, Glu, Gln, His, or Arg for Lys, substitution of Ile, Leu, Val, or Phe for Met, substitution of Trp, Tyr, Met, Ile, or Leu for Phe, substitution of Thr or Ala for Ser, substitution of Ser or Ala for Thr, substitution of Phe or Tyr for Trp, substitution of His, Phe, or Trp for Tyr, and substitution of Met, Ile, or Leu for Val. Furthermore, such substitution, deletion, insertion, and/or addition of amino acid residues as mentioned above can include a naturally occurring mutation due to an individual difference, or a difference of species of the organism from which the gene is derived (mutant or variant).

The fibroin-like protein may be a protein having an amino acid sequence showing a homology of 80% or higher, 90% or higher, 95% or higher, 97% or higher, or 99% or higher, to any of the aforementioned amino acid sequences of fibroin-like proteins, so long as the original function is maintained. In this description, “homology” can mean “identity”.

The fibroin-like protein may be a protein encoded by a DNA that is able to hybridize under stringent conditions with a probe that can be prepared from any of the aforementioned nucleotide sequences of fibroin-like protein genes, such as a sequence complementary to the whole sequence or a partial sequence of any of the aforementioned nucleotide sequences, so long as the original function is maintained. Such a probe can be prepared by PCR using oligonucleotides prepared on the basis of any of the aforementioned nucleotide sequences as the primers, and a DNA fragment including any of the aforementioned nucleotide sequences as the template. The term “stringent conditions” can refer to conditions under which a so-called specific hybrid is formed, and a non-specific hybrid is not formed. Examples of the stringent conditions can include those under which highly homologous DNAs hybridize to each other, for example, DNAs not less than 80% homologous, not less than 90% homologous, not less than 95% homologous, not less than 97% homologous, or not less than 99% homologous, hybridize to each other, and DNAs less homologous than the above do not hybridize to each other, or conditions of washing of typical Southern hybridization, i.e., conditions of washing once, or 2 or 3 times, at a salt concentration and temperature corresponding to 1 x SSC, 0.1% SDS at 60° C., 0.1×SSC, 0.1% SDS at 60° C., 0.1×SSC, 0.1% SDS at 68° C. Furthermore, for example, when a DNA fragment having a length of about 300 bp is used as the probe, the washing conditions of the hybridization can be, for example, 50° C., 2×SSC, and 0.1% SDS.

Furthermore, any codons in the fibroin-like protein gene may be replaced with respective equivalent codons. That is, the fibroin-like protein gene may also be a variant of any of the fibroin-like protein genes exemplified above due to the degeneracy of the genetic code. For example, the fibroin-like protein gene may be a gene modified so that it has optimal codons according to codon frequencies in the chosen host.

The percentage of the sequence identity between two sequences can be determined by, for example, using a mathematical algorithm. Non-limiting examples of such a mathematical algorithm can include the algorithm of Myers and Miller (1988) CABIOS 4:11-17, the local homology algorithm of Smith et al (1981) Adv. Appl. Math. 2:482, the homology alignment algorithm of Needleman and Wunsch (1970) J. Mol. Biol. 48:443-453, the method for searching homology of Pearson and Lipman (1988) Proc. Natl. Acad. Sci. 85:2444-2448, and an modified version of the algorithm of Karlin and Altschul (1990) Proc. Natl. Acad. Sci. USA 87:2264, such as that described in Karlin and Altschul (1993) Proc. Natl. Acad. Sci. USA 90:5873-5877.

By using a program based on such a mathematical algorithm, sequence comparison (i.e. alignment) for determining the sequence identity can be performed. The program can be appropriately executed by a computer. Examples of such a program can include, but is not limited to, CLUSTAL of PC/Gene program (available from Intelligenetics, Mountain View, Calif.), ALIGN program (Version 2.0), and GAP, BESTFIT, BLAST, FASTA, and TFASTA of Wisconsin Genetics Software Package, Version 8 (available from Genetics Computer Group (GCG), 575 Science Drive, Madison, Wis., USA). Alignment using these programs can be performed by using, for example, initial parameters. The CLUSTAL program is well described in Higgins et al. (1988) Gene 73:237-244 (1988), Higgins et al. (1989) CABIOS 5:151-153, Corpet et al. (1988) Nucleic Acids Res. 16:10881-90, Huang et al. (1992) CABIOS 8:155-65, and Pearson et al. (1994) Meth. Mol. Biol. 24:307-331.

In order to obtain a nucleotide sequence homologous to a target nucleotide sequence, in particular, for example, BLAST nucleotide search can be performed by using BLASTN program with score of 100 and word length of 12. In order to obtain an amino acid sequence homologous to a target protein, in particular, for example, BLAST protein search can be performed by using BLASTX program with score of 50 and word length of 3. See ncbi.nlm.nih.gov for BLAST nucleotide search and BLAST protein search. In addition, Gapped BLAST (BLAST 2.0) can be used in order to obtain an alignment including gap(s) for the purpose of comparison. In addition, PSI-BLAST (BLAST 2.0) can be used in order to perform repetitive search for detecting distant relationships between sequences. See Altschul et al. (1997) Nucleic Acids Res. 25:3389 for Gapped BLAST and PSI-BLAST. When using BLAST, Gapped BLAST, or PSI-BLAST, initial parameters of each program (e.g. BLASTN for nucleotide sequences, and BLASTX for amino acid sequences) can be used. Alignment can also be manually performed.

The sequence identity between two sequences is calculated as the ratio of residues matching in the two sequences when aligning the two sequences so as to fit maximally with each other.

The protein as described herein may be a protein having an amino acid sequence identical to that of any of the fibroin-like proteins exemplified above and conservative variants thereof, except that the protein as described herein contains a cell adhesion sequence. Also, a gene encoding the protein as described herein may be a gene having a nucleotide sequence identical to that of any of the fibroin-like protein genes exemplified above and conservative variants thereof, except that gene encoding the protein as described herein contains a nucleotide sequence encoding a cell adhesion sequence.

Specific examples of the protein as described herein can include the fibroin-like proteins used in the Examples section into which a cell adhesion sequence has been inserted. The nucleotide sequences of genes encoding the such proteins are shown in SEQ ID NOS: 3, 5, 26, 28, 35, 37, 39, 41, 43, 45, 47, 49, 51, and 53, and the amino acid sequences of such fibroin-like proteins encoded by these genes are shown in SEQ ID NOS: 4, 6, 27, 29, 36, 38, 40, 42, 44, 46, 48, 50, 52, and 54. That is, the protein as described herein may be, for example, a protein having the amino acid sequence shown in SEQ ID NO: 4, 6, 27, 29, 36, 38, 40, 42, 44, 46, 48, 50, 52, or 54. Specific examples of the protein as described herein also can include conservative variants of the cell adhesion sequence-inserted fibroin-like proteins used in the Examples section, provided that the variants each have a cell adhesion sequence. The aforementioned descriptions concerning conservative variants of the fibroin-like protein can be applied mutatis mutandis to conservative variants of the cell adhesion sequence-inserted fibroin-like protein. The expression “the original function is maintained” in relation to the cell adhesion sequence-inserted fibroin-like protein can mean that a variant of the protein has a scaffold function for a cell, such as a stem cell.

The term “gene” is not limited to DNA, but can include any polynucleotide, so long as it encodes a target protein. That is, the term “gene encoding the protein as described herein” may mean any polynucleotide encoding the protein as described herein. The gene encoding the protein as described herein may be DNA, RNA, or a combination thereof. The gene encoding the protein as described herein may be single-stranded or double-stranded. The gene encoding the protein as described herein may be a single-stranded DNA or a single-stranded RNA. The gene encoding the protein as described herein may be a double-stranded DNA, a double-stranded RNA, or a hybrid strand having a DNA strand and an RNA strand. The gene encoding the protein as described herein may contain both a DNA residue and an RNA residue in a single polynucleotide chain. When the gene encoding the protein as described herein contains RNA, the aforementioned descriptions concerning DNA, such as those concerning nucleotide sequences exemplified above, may be applied to RNA with appropriately changing wordings to those for RNA as required. The mode of the gene encoding the protein as described herein can be chosen according to various conditions such as use mode thereof.

Hereinafter, the structure of the protein as described herein will be specifically explained.

The protein as described herein includes a repetitive structure and a cell adhesion sequence. The repetitive structure has a repeating unit that repeats within the repetitive structure. Each repeating unit (the repeating unit in each repetition) has an Ala-rich region and an Ala-non-rich region mutually linked. In each repeating unit, the Ala-rich region is present at the N-terminus side and the Ala-non-rich region is present at the C-terminus side. That is, specifically, the repetitive structure consists of Ala-rich regions and Ala-non-rich regions linked alternately from the N-terminus toward the C-terminus.

The repetition number of the repeating unit is 3 to 15. The repetition number of the repeating unit may be, for example, 4 or more, 5 or more, 6 or more, 7 or more, 8 or more, 9 or more, or 10 or more. The repetition number of the repeating unit may be, for example, 12 or less, 10 or less, 8 or less, or 6 or less. The repetition number of the repeating unit may be, for example, within a range defined as a non-contradictory combination of the ranges described above. The repetition number of the repeating unit may be, specifically, for example, 3 to 12, 3 to 10, 3 to 8, 3 to 6, 5 to 15, 7 to 15, or 10 to 15.

The structure of the repeating unit may or may not be the same in each repetition. That is, the structure of the Ala-rich region may or may not be the same in each repetition. Also, the structure of the Ala-non-rich region may or may not be the same in each repetition. That is, the structure of the repeating unit, the structure of the Ala-rich region, and the structure of the Ala-non-rich region each may be selected independently in each repetition. That is, in each repeating unit, the Ala-rich region, and the Ala-non-rich region, a single kind of amino acid sequence may be used, or two or more kinds of amino acid sequences may be used in combination.

<Ala-Rich Region >

The term “Ala-rich region” can refer to a region having a high number of Ala residues, that is, a region containing Ala residues at a high ratio.

The length of the Ala-rich region can be 8 to 54 residues. That is, the Ala-rich region can be an amino acid sequence having a length of 8 to 54 residues. The length of the Ala-rich region may be, for example, 40 residues or shorter, 30 residues or shorter, 20 residues or shorter, 15 residues or shorter, or 10 residues or shorter. The length of the Ala-rich region may be, for example, within a range defined as a combination of the ranges described above. The length of the Ala-rich region may be, specifically, for example, 8 to 15 residues, or 8 to 10 residues. The length of the Ala-rich region may be, more specifically, for example, 9 residues.

The Ala-rich region may have Ala residues, or may have a combination of Ala residues and other amino acid residue(s), that is, a combination of Ala residues and amino acid residue(s) other than Ala residues.

The ratio of Ala residue in the Ala-rich region is 30% or more. The ratio of Ala residues in the Ala-rich region may be, for example, 40% or more, 50% or more, 60% or more, or 70% or more. The ratio of Ala residues in the Ala-rich region may be, for example, 100% or less, 95% or less, 90% or less, 85% or less, or 80% or less. The ratio of Ala residues in the Ala-rich region may be, for example, within a range defined as a combination of the ranges described above. The ratio of Ala residues in the Ala-rich region may be, specifically, for example, 30% to 100%, 50% to 90%, or 60% to 85%. The expression “ratio of Ala residue in an Ala-rich region” can refer to the ratio of the number of Ala residues in an Ala-rich region with respect to the total number of amino acid residues constituting the Ala-rich region. The ratio of any other amino acid residue in the Ala-rich region can be similarly calculated.

Examples of the other amino acid residue, that is, the amino acid residue other than Ala residue, can include Gly, Val, Leu, Ile, Ser, Thr, Cys, Met, Asn, Gln, Pro, Asp, Glu, Lys, Arg, His, Phe, Tyr, and Trp. Specific examples of the other amino acid residue can include, for example, Gly residue and Ser residue. The Ala-rich region may contain a single kind of other amino acid residue, or contain two or more kinds of other amino acid residues. The Ala-rich region, for example, may contain one or more Gly residues, may contain one or more Ser residues, or may contain one or more Gly residues and one or more Ser residues. Both the N-terminus and C-terminus of the Ala-rich region may each be, for example, an Ala residue, a Gly residue, or a Ser residue. Specifically, for example, the N-terminus of the Ala-rich region may be an Ala or Gly residue, and the C-terminus of the Ala-rich region may be an Ala or Ser residue. The ratio of Ala, Gly, and Ser residues in the Ala-rich region may be, for example, 50% or more, 60% or more, 70% or more, 80% or more, 90% or more, 95% or more, or 100%, in total.

In the Ala-rich region, in any stretch of 4 consecutive amino acid residues, there must be at least one Ala residue. That is, there are no sequences of 4 consecutive residues that do not contain an Ala residue in the Ala-rich region. Also, the Ala-rich region may contain at least two Ala residues in any stretch of 5, 6, or 7 consecutive amino acid residues in the region. That is, there may be no sequences of 5, 6, or 7 consecutive amino acid residues that do not contain an Ala residue or that contain only one Ala residue in the Ala-rich region.

Examples of the amino acid sequence of the Ala-rich region can include, for example, the amino acid sequence of a region satisfying such requirement(s) of the Ala-rich region as described above within the amino acid sequence of a fibroin-like protein. The fibroin-like protein is as described above. Examples of the region satisfying such requirement(s) of the Ala-rich region as described above within the amino acid sequence of SEQ ID NO: 2 can include the regions of amino acid numbers 53 to 61, 92 to 100, 121 to 129, 152 to 160, 186 to 194, 219 to 227, 258 to 266, 292 to 300, 326 to 334, 370 to 378, 439 to 447, and 498 to 506. Specific examples of the amino acid sequences of those regions satisfying the requirement(s) of the Ala-rich region can include, for example, GASAAAAAA (SEQ ID NO: 7), GSSAAAAAA (SEQ ID NO: 8), GASAASAAS (SEQ ID NO: 9), and GASAAAGAA (SEQ ID NO: 10). That is, the Ala-rich region may have, for example, any of the amino acid sequences of those regions satisfying the requirement(s) of the Ala-rich region (e.g. SEQ ID NO: 7, 8, 9, or 10).

The Ala-rich region may also be a variant of any of the amino acid sequences of those regions satisfying the requirement(s) of the Ala-rich region (e.g. SEQ ID NO: 7, 8, 9, or 10). The aforementioned descriptions concerning conservative variants of the fibroin-like protein can be applied similarly to variants of those regions satisfying the requirement(s) of the Ala-rich region. The Ala-rich region may be, for example, one having any of the amino acid sequences of those regions satisfying the requirement(s) of the Ala-rich region (e.g. SEQ ID NO: 7, 8, 9, or 10) including substitution, deletion, insertion, and/or addition of one or several amino acid residues at one or several positions. Although the number meant by the term “one or several” can differ depending on the length of those regions satisfying the requirement(s) of the Ala-rich region etc., it may be, for example, 1 to 5, 1 to 4, 1 to 3, 1 to 2, or 1. The Ala-rich region may also be, for example, one having an amino acid sequence having an identity of 80% or higher, 90% or higher, 95% or higher, 97% or higher, or 99% or higher, relative to the entire amino acid sequences of those regions satisfying the requirement(s) of the Ala-rich region (e.g. SEQ ID NO: 7, 8, 9, or 10). Variants of those regions satisfying the requirement(s) of the Ala-rich region are not particularly limited, so long as the amino acid sequences after modification satisfy such requirement(s) of the Ala-rich region as described above.

<Ala-Non-rich region >

The term “Ala-non-rich region” can refer to a region having a low ratio of Ala residues.

The length of the Ala-non-rich region can be 4 residues or longer. That is, the Ala-non-rich region can be an amino acid sequence having a length of 4 residues or longer. The length of the Ala-non-rich region may be, for example, 10 residues or longer, 15 residues or longer, 20 residues or longer, 30 residues or longer, 40 residues or longer, or 50 residues or longer. The length of the Ala-non-rich region may be, for example, 200 residues or shorter, 150 residues or shorter, 100 residues or shorter, 80 residues or shorter, 60 residues or shorter, or 40 residues or shorter. The length of the Ala-non-rich region may be, for example, within a range defined as a non-contradictory combination of the ranges described above. The length of the Ala-non-rich region may be, specifically, for example, 4 to 200 residues, 10 to 150 residues, or 15 to 100 residues. [000100] The Ala-non-rich region may or may not contain Ala residue(s). The ratio of Ala residues in the Ala-non-rich region is 20% or less. The ratio of Ala residues in the Ala-non-rich region may be, for example, 16% or less, 14% or less, 10% or less, 5% or less, or 0%. [000101] The Ala-non-rich region may contain, specifically, for example, one or more kinds of, e.g. all of, amino acid residues such as Gly, Ser, Gln, Pro, and Tyr. The ratio of Gly, Ser, Gln, Pro, and Tyr residues in the Ala-non-rich region may be, for example, 50% or more, 60% or more, 70% or more, 80% or more, 90% or more, 95% or more, or 100%, in total. The Ala-non-rich region may contain sequences of 4 consecutive amino acid residues that contain no Ala residue. The Ala-non-rich region may contain, for example, sequences of 4 consecutive amino acid residues that contain no Ala residue at each of the termini (i.e. N-terminus and C-terminus).

Examples of the amino acid sequence of the Ala-non-rich region can include, for example, the amino acid sequence of a region satisfying such requirement(s) of the Ala-non-rich region as described above within the amino acid sequence of a fibroin-like protein. The fibroin-like protein is as described above. Examples of the region satisfying such requirement(s) of the Ala-non-rich region as described above within the amino acid sequence of SEQ ID NO: 2 can include the regions of amino acid numbers of 62 to 91, 101 to 120, 130 to 151, 161 to 185, 195 to 218, 228 to 257, 267 to 291, 301 to 325, 335 to 369, 379 to 438, 448 to 497, and 507 to 536. That is, the Ala-non-rich region may have, for example, any of the amino acid sequences of those regions satisfying the requirement(s) of the Ala-non-rich region. The Ala-non-rich region may also have, for example, a partial sequence of any of the amino acid sequences of the Ala-non-rich region exemplified above (e.g. the amino acid sequences of those regions satisfying the requirement(s) of the Ala-non-rich region). The partial sequence is not particularly limited, so long as it satisfies the requirement(s) of the Ala-non-rich region as described above. Examples of the partial sequence can include, for example, a partial sequence of any of the amino acid sequences of the Ala-non-rich region described above, which partial sequence satisfies the length of the Ala-non-rich region described above. Examples of the partial sequence also can include, for example, a partial sequence of any of the amino acid sequences of the Ala-non-rich region described above, which partial sequence has a length of 30% or longer, 50% or longer, 70% or longer, or 90% or longer of the length of any of those amino acid sequences of the Ala-non-rich region. Furthermore, specific examples of motifs contained in those regions satisfying the requirement(s) of the Ala-non-rich region can include, for example, GGYGPGSGQQG (SEQ ID NO: 11), GGNGPGSGQQG (SEQ ID NO: 12), GGYGPGYGQQG (SEQ ID NO: 13), GGYGPGSGQG (SEQ ID NO: 14), PGQQG (SEQ ID NO: 15), AGQQG (SEQ ID NO: 16), SGQQG (SEQ ID NO: 17), PSQQG (SEQ ID NO: 18), PGGQG (SEQ ID NO: 19), PYGP (SEQ ID NO: 20), and AYGP (SEQ ID NO: 21). The motifs shown in SEQ ID NOS: 11 to 14 can also collectively be referred to as “motif A”, the motifs shown in SEQ ID NOS: 15 to 19 can also be collectively referred to as “motif B”, and the motifs shown in SEQ ID NOS: 20 to 21 can also be collectively referred to as “motif C”. That is, the Ala-non-rich region may have, more typically, for example, one or more kinds of these motifs. The Ala-non-rich region, specifically, for example, may have motifs A and B, and may further have motif C. When the Ala-non-rich region has two or more kinds of these motifs, the order of the motifs is not particularly limited. The Ala-non-rich region may have, for example, motifs A and B or motifs A, B, and C from the N-terminus toward the C-terminus. The Ala-non-rich region may have only one unit each in any one or more of these motifs, or may have two or more units in any one or more of these motifs. When the Ala-non-rich region has two or more units in any one motif, the units of the motif may or may not be arranged in tandem in the Ala-non-rich region. The Ala-non-rich region may have, for example, only one unit of motif B, or may have two or more units of motif B. The number of motif B contained in one Ala-non-rich region, for example, may be 1 or more, 2 or more, 3 or more, 5 or more, or 7 or more, may be 15 or less, 10 or less, 7 or less, or 5 or less, or may be within a range defined as a non-contradictory combination thereof.

The Ala-non-rich region may also be a variant of any of the amino acid sequences of the regions satisfying the requirement(s) of the Ala-non-rich region, a variant of any of partial sequences thereof, or a variant of any of motifs contained therein. The aforementioned descriptions concerning conservative variants of the fibroin-like protein can be applied similarly to variants of the amino acid sequences of the regions satisfying the requirement(s) of the Ala-non-rich region, variants of partial sequences thereof, or variants of motifs contained therein. The Ala-non-rich region may be, for example, one having any of the amino acid sequences of those regions satisfying the requirement(s) of the Ala-non-rich region, any partial sequences thereof, or any motifs contained therein including substitution, deletion, insertion, and/or addition of one or several amino acid residues at one or several positions. Although the number meant by the term “one or several” can differ depending on the length of the amino acid sequences of those regions satisfying the requirement(s) of the Ala-non-rich region, partial sequences thereof, or motifs contained therein etc., it may be, for example, 1 to 20, 1 to 15, 1 to 10, 1 to 5, 1 to 4, 1 to 3, 1 to 2, or 1. The Ala-non-rich region may also be, for example, one having an amino acid sequence having an identity of 80% or higher, 90% or higher, 95% or higher, 97% or higher, 99% or higher, relative to the entire amino acid sequence of the regions satisfying the requirement(s) of the Ala-non-rich region, entire partial sequences thereof, or the entire motifs contained therein. Variants of the amino acid sequences of those regions satisfying the requirement(s) of the Ala-non-rich region, variants of partial sequences thereof, or variants of motifs contained therein are not particularly limited, so long as the amino acid sequences after modification satisfy such requirement(s) of the Ala-non-rich region as described above.

<Cell Adhesion Sequence>

The term “cell adhesion sequence” can refer to an amino acid sequence capable of imparting a scaffold function for a cell, such as a stem cell, to a protein such as a fibroin-like protein. The term “cell adhesion sequence” specifically can refer to an amino acid sequence capable of imparting a scaffold function for a cell, such as a stem cell, to a protein such as a fibroin-like protein due to insertion of the amino acid sequence into the protein. Such a characteristic, that is, a characteristic capable of imparting a scaffold function for a cell, such as a stem cell, to a protein such as a fibroin-like protein, can also be referred to as “characteristic as a cell adhesion sequence”.

The cell adhesion sequence can be an amino acid sequence having a length of 3 to 18 residues containing RGD (Arg-Gly-Asp). That is, the length of the cell adhesion sequence can be 3 to 18 residues. The cell adhesion sequence may be a sequence that is only RGD, or may be a larger sequence that includes RGD. Examples of the cell adhesion sequence can include a partial amino acid sequence of a cell adhesion protein (i.e. a part of the amino acid sequence of a cell adhesion protein), which partial amino acid sequence contains RGD, i.e. an RGD sequence of a cell adhesion protein. Examples of the cell adhesion sequence also can include a combination of partial amino acid sequences of cell adhesion protein(s), which combination contains RGD. Specific examples of the cell adhesion protein can include, for example, fibronectin, vitronectin, collagen, osteopontin, and laminin. Specific examples of the RGD sequence of a cell adhesion protein can include, for example, GRGDSP (SEQ ID NO: 22), VTGRGDSPAS (SEQ ID NO: 23), PQVTRGDVFTM (SEQ ID NO: 24), VTRGDVF (SEQ ID NO: 25), GRGDNP (SEQ ID NO: 30), and GAAGRGDSPAAGY (SEQ ID NO: 31). That is, the cell adhesion sequence may have, for example, any of those amino acid sequences (e.g. SEQ ID NO: 22, 23, 24, 25, 30, or 31).

The cell adhesion sequence may also be a variant of any of those amino acid sequences (e.g. a partial amino acid sequence of a cell adhesion protein, such as SEQ ID NO: 22, 23, 24, 25, 30, or 31), so long as RGD is conserved. The aforementioned descriptions concerning conservative variants of the fibroin-like protein can be similarly applied to variants of those amino acid sequences. The cell adhesion sequence may be, for example, one having any of those amino acid sequences (e.g. a partial amino acid sequence of a cell adhesion protein, such as SEQ ID NO: 22, 23, 24, 25, 30, or 31) including substitution, deletion, insertion, and/or addition of one or several amino acid residues at one or several positions other than the position of RGD. Although the number meant by the term “one or several” can differ depending on the length of those regions satisfying the requirement(s) of the cell adhesion sequence etc., it may be, for example, 1 to 5, 1 to 4, 1 to 3, 1 to 2, or 1. The cell adhesion sequence may also be, for example, one having an amino acid sequence showing an identity of 80% or higher, 90% or higher, 95% or higher, 97% or higher, or 99% or higher, relative to the entire amino acid sequence of any of those regions satisfying the requirement(s) of the cell adhesion sequence (e.g. a partial amino acid sequence of a cell adhesion protein, such as SEQ ID NO: 22, 23, 24, 25, 30, or 31), provided that RGD is conserved. Variants of those amino acid sequences are not particularly limited, so long as RGD is conserved, and the variants have the characteristic as a cell adhesion sequence (i.e. the variants are capable of imparting a scaffold function for a cell, such as a stem cell, to a protein such as a fibroin-like protein).

Whether or not a certain amino acid sequence has the characteristic as a cell adhesion sequence can be confirmed by, for example, confirming whether or not a fibroin-like protein that has a certain amino acid sequence inserted has a scaffold function for a cell, such as a stem cell. Whether or not such a protein has a scaffold function for a cell, such as a stem cell, can be confirmed by, for example, culturing a cell, such as a stem cell, using a culture vessel having a cell culture surface coated with the protein. That is, when proliferation of a cell, such as a stem cell, is improved due to coating of the cell culture surface with the protein, it can be concluded that the protein shows a scaffold function for a cell, such as a stem cell.

The protein as described herein contains the cell adhesion sequence in the repetitive structure. That is, the cell adhesion sequence has been inserted into the repetitive structure. In other words, the cell adhesion sequence can be inserted into the repetitive structure that is in the protein as described herein. The cell adhesion sequence can be inserted (contained) in, specifically, one or more repeating units that make up the repetitive structure. The cell adhesion sequence can be inserted (contained) in, for example, 2 or more, 3 or more, 4 or more, 5 or more, 6 or more, or 7 or more of the repeating units that make up the repetitive structure. The cell adhesion sequence can be inserted (contained) in, for example, all of the repeating units constituting the repetitive structure. One repeating unit may have only one unit of the cell adhesion sequence, or may have two or more units of the cell adhesion sequence. When one repeating unit has two or more units of the cell adhesion sequence, the units of the cell adhesion sequence may or may not be arranged in tandem in the repeating unit. The number of the cell adhesion sequence present in one repeating unit, for example, may be 1 or more, 2 or more, or 3 or more, and may be 5 or less, 3 or less, or 2 or less, or may be within a range defined as a non-contradictory combination thereof. The number of the cell adhesion sequence present in one repeating unit may be, specifically, for example, 1 to 3. The total number of the cell adhesion sequence present in the protein as described herein, for example, may be 1 or more, 2 or more, 3 or more, 4 or more, 5 or more, 6 or more, or 7 or more, and may be 50 or less, 40 or less, 30 or less, 25 or less, 20 or less, 15 or less, or 10 or less, or may be within a range defined as a combination thereof. The total number of the cell adhesion sequence present in the repetitive structure may be, specifically, for example, 1 to 50, 3 to 30, or 5 to 15. When the protein as described herein contains two or more cell adhesion sequences, the structures of the cell adhesion sequences each may be independently selected. That is, as the cell adhesion sequence, a single kind of cell adhesion sequence may be used, or two or more kinds of cell adhesion sequences may be used in combination.

The position of the cell adhesion sequence in the repeating unit is not particularly limited. Each cell adhesion sequence may be inserted, for example, in the Ala-non-rich region, or in the linkage site between the Ala-rich region and the Ala-non-rich region. Examples of the Ala-non-rich region into which the cell adhesion sequence can be inserted can include, for example, the regions of amino acid numbers of 62 to 91, 101 to 120, 130 to 151, 161 to 185, 195 to 218, 228 to 257, 267 to 291, 301 to 325, 335 to 369, 379 to 438, 448 to 497, and 507 to 536 within the amino acid sequence of SEQ ID NO: 2. Specific examples of the Ala-non-rich region into which the cell adhesion sequence can be inserted can include, for example, the positions between amino acid numbers 109 and 110, 170 and 171, 203 and 204, 276 and 277, 310 and 311, 403 and 404, and 472 and 473 within the amino acid sequence of SEQ ID NO: 2. Examples of the linkage site between the Ala-rich region and the Ala-non-rich region into which the cell adhesion sequence can be inserted can include, for example, the sites between amino acid numbers 61 and 62, 91 and 92, 100 and 101, 120 and 121, 129 and 130, 151 and 152, 160 and 161, 185 and 186, 194 and 195, 218 and 219, 227 and 228, 257 and 258, 266 and 267, 291 and 292, 300 and 301, 325 and 326, 334 and 335, 369 and 370, 378 and 379, 438 and 439, 447 and 448, 497 and 498, and 506 and 507 within the amino acid sequence of SEQ ID NO: 2. The cell adhesion sequence may be further inserted into another region, as well as in the Ala-non-rich region and/or in the linkage site between the Ala-rich region and the Ala-non-rich region. Examples of the other region can include the Ala-rich region, and N-terminal and C-terminal regions described below.

<Other Regions>

The protein as described herein may further contain an N-terminal region before the first repeating unit, i.e. before the repetition structure. The amino acid sequence of the N-terminal region is not particularly limited. The length of the N-terminal region, for example, may be 1 residue or longer, 3 residues or longer, 5 residues or longer, 10 residues or longer, 30 residues or longer, 50 residues or longer, or 100 residues or longer, and may be 200 residues or shorter, 150 residues or shorter, 100 residues or shorter, 80 residues or shorter, 60 residues or shorter, or 40 residues or shorter, or may be within a range defined as a non-contradictory combination thereof. The length of the N-terminal region may be, specifically, for example, 1 to 200 residues. The N-terminal region may include an amino acid sequence satisfying the requirement(s) of the Ala-non-rich region.

The protein as described herein may further contain a C-terminal region after the last repeating unit, i.e. after the repetition structure. The amino acid sequence of the C-terminal region is not particularly limited. The length of the C-terminal region, for example, may be 1 residue or longer, 3 residues or longer, 5 residues or longer, 10 residues or longer, 30 residues or longer, 50 residues or longer, or 100 residues or longer, and may be 200 residues or shorter, 150 residues or shorter, 100 residues or shorter, 80 residues or shorter, 60 residues or shorter, or 40 residues or shorter, or may be within a range defined as a non-contradictory combination thereof. The length of the C-terminal region may be, specifically, for example, 1 to 200 residues. The C-terminal region may include an amino acid sequence satisfying the requirement(s) of the Ala-rich region. Furthermore, the C-terminal region may include, for example, an amino acid sequence having a homology of 90% or higher to an amino acid sequence around the C-terminus of the fibroin of spider at the C-terminus, in addition to the sequence similar to the repetitive sequence of fibroin. Examples of the amino acid sequence around the C-terminus of the fibroin of spider can include, for example, the amino acid sequence of the C-terminus 50 residues of the fibroin of spider, the amino acid sequence of the C-terminus 50 residues of the same of which the C-terminus 20 residues are removed, and the amino acid sequence of the C-terminus 50 residues of the same of which the C-terminus 29 residues are removed. Specific examples of the amino acid sequence around the C-terminus of the fibroin of spider can include, for example, the amino acid sequence of the positions 587 to 636 (C-terminus 50 residues), the amino acid sequence of the positions 587 to 616, and the amino acid sequence of the positions 587 to 607 of ADF3 of Araneus diadematus (partial; NCBI AAC47010.1 GI: 1263287). Specific examples of the protein as described herein including the amino acid sequence of the positions 587 to 636 of ADF3 of Araneus diadematus (partial; NCBI AAC47010.1 GI: 1263287) can include, for example, a protein having an amino acid sequence of SEQ ID NO: 4, 6, 27, 29, 36, 38, 40, 42, 44, 46, 48, 50, 52, or 54.

Furthermore, the N-terminal and C-terminal regions each may contain a functional sequence, such as a peptide tag, and a recognition sequence for a protease. As the functional sequence, one kind of functional sequence may be used, or two or more kinds of functional sequences may be used in combination. Specific examples of the peptide tag can include an His tag, FLAG tag, GST tag, Myc tag, MBP (maltose binding protein), CBP (cellulose binding protein), TRX (thioredoxin), GFP (green fluorescent protein), HRP (horseradish peroxidase), ALP (alkaline phosphatase), and Fc region of antibody. The peptide tag can be used for, for example, detection and purification of the expressed protein. Specific examples of the recognition sequence for a protease can include the recognition sequence for the HRV3C protease, the recognition sequence for the factor Xa protease, and the recognition sequence for the proTEV protease. The recognition sequence for a protease can be used for, for example, cleavage of the expressed protein. Specific examples of the protein as described herein having an His-tag and an HRV3C protease recognition sequence at the N-terminus can include, for example, a protein having an amino acid sequence of SEQ ID NO: 4, 6, 36, 42, 48, or 50.

<1-2> Production of the Protein of the Present Invention

The protein as described herein can be produced by making a host having a gene encoding the protein as described herein express the protein as described herein. The phrase “having a gene encoding the protein as described herein” can also be referred to as “having the protein as described herein”. That is, for example, a host having a gene encoding the protein as described herein can also be referred to as a “host having the protein as described herein”. In addition, expression of a gene encoding the protein as described herein can also be referred to as “expression of the protein as described herein”. Alternatively, the protein as described herein can also be produced by expressing the same using a cell-free protein synthesis system.

The host having the gene encoding the protein as described herein can be obtained by introducing the gene encoding the protein as described herein into an appropriate host.

The host into which the gene encoding the protein as described herein can be introduced is not particularly limited, so long as it is able to express the protein as described herein. Examples of the host can include, for example, bacteria, actinomycetes, yeast, fungi, plant cells, insect cells, and animal cells. Specific examples of the host can include microorganisms such as bacteria and yeast, including gram-negative bacteria and gram-positive bacteria. Examples of the gram-negative bacteria can include, for example, bacteria belonging to the family Enterobacteriaceae, such as Escherichia bacteria, Enterobacter bacteria, and Pantoea bacteria. Examples of the gram-positive bacteria can include Bacillus bacteria, and coryneform bacteria such as Corynebacterium bacteria. Examples of the Escherichia bacteria can include, for example, Escherichia coli. Examples of the coryneform bacteria can include, for example, Corynebacterium glutamicum and Corynebacterium ammoniagenes (Corynebacterium stationis). Specific examples of Escherichia coli can include, for example, Escherichia coli K-12 strains such as the W3110 strain (ATCC 27325) and MG1655 strain (ATCC 47076); Escherichia coli K5 strain (ATCC 23506); Escherichia coli B strains such as the BL21(DE3) strain and a recA′ strain thereof BLR(DE3); and derivative strains of these.

These strains are available from, for example, the American Type Culture Collection (Address: P.O. Box 1549, Manassas, Va. 20108, United States of America). That is, registration numbers are given to the respective strains, and the strains can be ordered by using these registration numbers (refer to atcc.org). The registration numbers of the strains are listed in the catalogue of the American Type Culture Collection. The BL21(DE3) strain is available from, for example, Life Technologies (product number C6000-03). The BLR(DE3) strain is available from, for example, Merck Millipore (product number 69053).

The gene encoding the protein as described herein can be obtained by, for example, inserting a nucleotide sequence encoding the cell adhesion sequence into a fibroin-like protein gene. The fibroin-like protein gene can be obtained by cloning from an organism having the fibroin-like protein gene, such as a spider or a silkworm. For the cloning, a nucleic acid such as genomic DNA or cDNA containing the gene can be used. The obtained fibroin-like protein gene can be used as a base of the gene encoding the protein as described herein as it is, or after being modified as required. Modification of a gene, such as insertion of the nucleotide sequence encoding the cell adhesion sequence, can be carried out by a known technique. For example, an objective mutation can be introduced into a target site of DNA by the site-specific mutation method. That is, for example, a coding region of a gene can be modified by the site-specific mutagenesis method so that a specific site of the encoded protein can include substitution, deletion, insertion, and/or addition of amino acid residues. Examples of the site-specific mutagenesis method can include a method of using PCR (Higuchi, R., 61, in PCR Technology, Erlich, H. A. Eds., Stockton Press (1989); Carter P., Meth., in Enzymol., 154, 382 (1987)), and a method of using a phage (Kramer, W. and Frits, H. J., Meth. in Enzymol., 154, 350 (1987); Kunkel, T. A. et al., Meth. in Enzymol., 154, 367 (1987)). The gene encoding the protein as described herein can also be obtained by, for example, chemical synthesis (Gene, 60(1), 115-127 (1987)).

Methods for introducing the gene encoding the protein as described herein into a host are not particularly limited. It is sufficient that the gene encoding the protein as described herein is harbored in the host in such a manner that it can be expressed under control of a promoter that functions in the host. In the host, the gene encoding the protein as described herein may exist on a vector autonomously replicable out of the chromosome such as a plasmid, or may be introduced into the chromosome. The host may have only one copy of the gene encoding the protein as described herein, or may have two or more copies of the gene encoding the protein as described herein. The host may have only one kind of gene encoding the protein as described herein, or may have two or more kinds of genes encoding the protein as described herein.

The promoter for expressing the gene encoding the protein as described herein is not particularly limited, so long as it is a promoter that functions in the host. The term “promoter that functions in a host” can refer to a promoter that has promoter activity in the host. The promoter may be a promoter derived from the host, or can be a heterologous promoter. The promoter may be the native promoter of the gene encoding the protein as described herein, or may be a promoter of another gene. The promoter may be a promoter stronger than the native promoter of the gene encoding the protein as described herein. Specific examples of strong promoters that function in Enterobacteriaceae bacteria, such as Escherichia coli, can include, for example, T7 promoter, trp promoter, trc promoter, lac promoter, tac promoter, tet promoter, araBAD promoter, rpoH promoter, PR promoter, and PL promoter. Examples of strong promoters that function in coryneform bacteria can include the artificially modified P54-6 promoter (Appl. Microbiol. Biotechnol., 53, 674-679 (2000)), pta, aceA, aceB, adh, and amyE promoters inducible in coryneform bacteria with acetic acid, ethanol, pyruvic acid, or the like, cspB, SOD, and tuf (EF-Tu) promoters, which are potent promoters capable of providing a large expression amount in coryneform bacteria (Journal of Biotechnology, 104 (2003) 311-323; Appl. Environ. Microbiol., 2005 Dec; 71 (12):8587-96), as well as lac promoter, tac promoter, and trc promoter. Furthermore, as the stronger promoter, a highly-active type of an existing promoter may also be obtained by using various reporter genes. For example, by making the -35 and -10 regions in a promoter region closer to the consensus sequence, the activity of the promoter can be enhanced (WO00/18935). Examples of highly active-type promoters can include various tac-like promoters (Katashkina JI et al., Russian Federation Patent Application No. 2006134574) and pnlp8 promoter (WO2010/027045). Methods for evaluating the strength of promoters and examples of strong promoters are described in the paper of Goldstein et al. (Prokaryotic Promoters in Biotechnology, Biotechnol. Annu. Rev., 1, 105-128 (1995)), and so forth.

A terminator for termination of gene transcription may be located downstream of the gene encoding the protein as described herein. The terminator is not particularly limited so long as it functions in the host. The terminator may be a terminator derived from the host, or a heterogenous terminator. The terminator may be the native terminator of the gene encoding the protein as described herein, or a terminator of another gene. Specific examples of the terminator can include, for example, T7 terminator, T4 terminator, fd phage terminator, tet terminator, and trpA terminator.

The gene encoding the protein as described herein can be introduced into a host, for example, by using a vector containing the gene. A vector containing the gene encoding the protein as described herein can also be referred to as expression vector or recombinant vector for the gene encoding the protein as described herein. The expression vector for the gene encoding the protein as described herein can be constructed by, for example, ligating a DNA fragment containing the gene encoding the protein as described herein with a vector that functions in the host. By transforming the host with the expression vector for the gene encoding the protein as described herein, a transformant into which the vector has been introduced can be obtained, i.e. the gene can be introduced into the host. As the vector, a vector autonomously replicable in the cell of the host can be used. The vector can be a multi-copy vector. Furthermore, the vector can have a marker such as an antibiotic resistance gene for selection of transformant. Furthermore, the vector may have a promoter and/or terminator for expressing the introduced gene. The vector may be, for example, a vector derived from a bacterial plasmid, a vector derived from a yeast plasmid, a vector derived from a bacteriophage, cosmid, phagemid, or the like. Specific examples of vectors autonomously replicable in Enterobacteriaceae bacteria such as Escherichia coli can include, for example, pUC19, pUC18, pHSG299, pHSG399, pHSG398, pBR322, pSTV29 (all of these are available from Takara Bio), pACYC184, pMW219 (NIPPON GENE), pTrc99A (Pharmacia), pPROK series vectors (Clontech), pKK233-2 (Clontech), pET series vectors (Novagen), pQE series vectors (QIAGEN), pACYC, and the broad host spectrum vector RSF1010. Specific examples of vectors autonomously replicable in coryneform bacteria can include, for example, pHM1519 (Agric. Biol. Chem., 48, 2901-2903 (1984)); pAM330 (Agric. Biol. Chem., 48, 2901-2903 (1984)); plasmids obtained by improving these and having a drug resistance gene; plasmid pCRY30 described in Japanese Patent Laid-open (Kokai) No. 3-210184; plasmids pCRY21, pCRY2KE, pCRY2KX, pCRY31, pCRY3KE, and pCRY3KX described in Japanese Patent Laid-open (Kokai) No. 2-72876 and U.S. Pat. No. 5,185,262; plasmids pCRY2 and pCRY3 described in Japanese Patent Laid-open (Kokai) No. 1-191686; pAJ655, pAJ611, and pAJ1844 described in Japanese Patent Laid-open (Kokai) No. 58-192900; pCG1 described in Japanese Patent Laid-open (Kokai) No. 57-134500; pCG2 described in Japanese Patent Laid-open (Kokai) No. 58-35197; and pCG4 and pCG11 described in Japanese Patent Laid-open (Kokai) No. 57-183799. When the expression vector is constructed, for example, the gene encoding the protein as described herein with its native promoter region may be incorporated into a vector, a coding region of the protein as described herein ligated downstream from a promoter as described above may be incorporated into a vector, or a coding region of the protein as described herein may be incorporated into a vector downstream from a promoter originally existing in the vector.

Vectors, promoters, and terminators available in various microorganisms are disclosed in detail in “Fundamental Microbiology Vol. 8, Genetic Engineering, KYORITSU SHUPPAN CO., LTD, 1987”, and those can be used.

The gene encoding the protein as described herein can also be introduced into, for example, the chromosome of a host. A gene can be introduced into a chromosome, for example, by using homologous recombination (Miller, J. H., Experiments in Molecular Genetics, 1972, Cold Spring Harbor Laboratory). Examples of gene transfer method utilizing homologous recombination can include, for example, a method of using a linear DNA such as Red-driven integration (Datsenko, K. A., and Wanner, B. L., Proc. Natl. Acad. Sci. USA, 97:6640-6645 (2000)), a method of using a plasmid containing a temperature sensitive replication origin, a method of using a plasmid capable of conjugative transfer, a method of using a suicide vector not having a replication origin that functions in a host, and a transduction method using a phage. Only one copy of the gene may be introduced, or two or more copies of the gene may be introduced. For example, by performing homologous recombination using a sequence which is present in multiple copies on a chromosome as a target, multiple copies of a gene can be introduced into the chromosome. Examples of such a sequence which is present in multiple copies on a chromosome can include repetitive DNAs, and inverted repeats located at the both ends of a transposon. Homologous recombination may also be performed by using an appropriate sequence on a chromosome such as a gene unnecessary for carrying out the present invention as a target. Furthermore, a gene can also be randomly introduced into a chromosome by using a transposon or Mini-Mu (Japanese Patent Laid-open (Kokai) No. 2-109985, U.S. Pat. No. 5,882,888, EP 805867 B1). When the gene is introduced into a chromosome, for example, the gene encoding the protein as described herein with its native promoter region may be incorporated into a chromosome, a coding region of the protein as described herein ligated downstream from a promoter as described above may be incorporated into a chromosome, or a coding region of the protein as described herein may be incorporated into a chromosome downstream from a promoter originally existing in the chromosome.

Introduction of a gene into a chromosome can be confirmed by, for example, Southern hybridization using a probe having a sequence complementary to the entire gene or a part thereof, or PCR using primers prepared on the basis of the nucleotide sequence of the gene.

Methods for transformation are not particularly limited, and conventionally known methods can be used. Examples of transformation method can include, for example, a method of treating recipient cells with calcium chloride so as to increase permeability thereof for DNA, which has been reported for the Escherichia coli K-12 strain (Mandel, M. and Higa, A., J. Mol. Biol., 1970, 53, 159-162), a method of preparing competent cells from cells which are in the growth phase, followed by introducing DNA, which has been reported for Bacillus subtilis (Duncan, C. H., Wilson, G. A. and Young, F. E., 1977, Gene, 1:153-167), and so forth. Alternatively, as the transformation method, there can also be used a method of making DNA-recipient cells into protoplasts or spheroplasts, which can easily take up recombinant DNA, and then introducing a recombinant DNA into the DNA-recipient cells, which is known to be applicable to Bacillus subtilis, actinomycetes, and yeasts (Chang, S. and Choen, S. N., 1979, Mol. Gen. Genet., 168:111-115; Bibb, M. J., Ward, J. M. and Hopwood, O. A., 1978, Nature, 274:398-400; Hinnen, A., Hicks, J. B. and Fink, G. R., 1978, Proc. Natl. Acad. Sci. USA, 75:1929-1933). Furthermore, as the transformation method, the electric pulse method reported for coryneform bacteria (Japanese Patent Laid-open (Kokai) No. 2-207791) can also be used.

By culturing the host having the gene encoding the protein as described herein, the protein as described herein can be expressed. During the culture, induction of gene expression is carried out as required. Conditions for culture of the host and induction of gene expression may be appropriately chosen depending on various conditions such as the type of marker, the type of promoter, and the type of the host. The culture medium used for the culture is not be particularly limited, so long as the host can proliferate in the culture medium and express the protein as described herein. As the culture medium, for example, a usual culture medium that contains a carbon source, nitrogen source, sulfur source, inorganic ions, and other organic components as required can be used.

Examples of the carbon source can include saccharides such as glucose, fructose, sucrose, molasses, and starch hydrolysate, alcohols such as glycerol and ethanol, and organic acids such as fumaric acid, citric acid, and succinic acid.

Examples of the nitrogen source can include inorganic ammonium salts such as ammonium sulfate, ammonium chloride, and ammonium phosphate, organic nitrogen such as soybean hydrolysate, ammonia gas, and aqueous ammonia.

Examples of the sulfur source can include inorganic sulfur compounds, such as sulfates, sulfites, sulfides, hyposulfites, and thiosulfates.

Examples of the inorganic ions can include calcium ion, magnesium ion, manganese ion, potassium ion, iron ion, and phosphoric acid ion.

Examples of the other organic components can include organic trace amount nutrients. Examples of the organic trace amount nutrients can include required substances such as vitamin Bi, yeast extract containing such substances, and so forth.

The culture temperature may be, for example, 20 to 45° C., 24 to 45° C., or 30 to 37° C. The culture can be carried out as an aerobic culture. Upon aeration of the culture, the oxygen concentration may be adjusted to, for example, 5 to 50%, or 20 to 40%, with respect to the saturated concentration. The pH of the culture medium may be 5 to 9. To adjust the pH, inorganic or organic acidic or alkaline substances such as calcium carbonate, ammonia gas, and aqueous ammonia can be used. The culture period may be, for example, 10 to 120 hours.

Particularly when using Escherichia coli as a host, it is expected that the protein as described herein can be efficiently produced by reducing accumulation of an organic acid at the time of inducing the expression of the protein as described herein (WO2015/178465), or by reducing cell growth after inducing the expression of the protein as described herein (WO2015/178466).

By culturing the host having the gene encoding the protein as described herein as described above, the protein as described herein is accumulated in the culture medium and/or cells of the host. The protein as described herein can be accumulated as, for example, inclusion bodies in the cells.

The protein as described herein can be collected and quantified by, for example, known methods for collecting and quantifying a heterogeneously expressed protein (see, for example, “Lecture of New Chemical Experiments, Protein VI, Synthesis and Expression”, Ed. By Japanese Biochemical Society, Tokyo Kagaku Dojin, 1992, pp.183-184).

Hereinafter, a procedure for collecting and quantifying the protein as described herein will be exemplified for when the protein is accumulated as inclusion bodies in the cells. First, the cells are collected from the culture medium by centrifugation, and then suspended in a buffer. The cell suspension is subjected to such a treatment as ultrasonication or French press to disrupt the cells. Before disrupting the cells, lysozyme may be added to the cell suspension at a final concentration of 0 to 200 mg/l, and the suspension may be incubated on ice for 30 minutes to 20 hours. Then, an insoluble fraction is obtained from the disrupted cell suspension as precipitates by low speed centrifugation (6000 to 15000 rpm, 5 to 10 minutes, 4° C.). The insoluble fraction is appropriately washed with a buffer as required. The number of times of washing is not be particularly limited, and may be, for example, once, twice, or 3 times or more. By suspending the insoluble fraction in a buffer, a suspension of the protein as described herein is obtained. As the buffer for suspending the cells or the protein as described herein, a buffer in which the protein as described herein shows a low solubility can be used. Examples of such a buffer can include, for example, a Tris-HCl buffer containing NaCl. The pH of the buffer may be, for example, usually 4 to 12, or 6 to 9. A solution of the protein as described herein can also be obtained by dissolving the insoluble fraction in an SDS solution or urea solution. The protein as described herein collected may contain such components as bacterial cells, culture medium components, and bacterial metabolic by-products, in addition to the protein as described herein. The protein as described herein may be purified to a desired degree. The protein as described herein can be purified by known methods used for purification of proteins. Examples of such methods can include, for example, ammonium sulfate fractionation, ion exchange chromatography, hydrophobic chromatography, affinity chromatography, gel filtration chromatography, isoelectric precipitation, and dialysis. These methods may be independently used, or may be used in an appropriate combination. The amount of the protein as described herein can be determined, for example, as follows: a sample containing the protein as described herein such as suspension or solution is subjected to SDS-PAGE, and stained, and then, the amount of the protein as described herein can be determined on the basis of intensity of a band at the position corresponding to the molecular weight of the objective protein as described herein. The staining can be performed by CBB staining, fluorescence staining, silver staining, or the like. For the quantification, proteins of known concentrations can be used as the standards. Examples of such proteins can include, for example, albumin, and the protein as described herein of which the concentration has been separately determined.

The intended use of the protein as described herein is not particularly limited. The protein as described herein may be used for, for example, medical use or research use. The protein as described herein shows a scaffold function for a cell, such as a stem cell. Hence, the protein as described herein may be used for, for example, culturing a cell, such as a stem cell. The protein as described herein may be used as, specifically, for example, a scaffold for cell culture. The protein as described herein may be used as, for example, a component of an apparatus such as an apparatus for cell culture. The protein as described herein may be used in such a manner that, specifically, for example, a surface of an apparatus such as a cell culture vessel is coated with the protein. The descriptions below concerning the apparatus as described herein and the method as described herein can be applied similarly to an apparatus and cell culture.

The protein as described herein can be used, for example, in combination with another protein (i.e. a protein other than the protein as described herein). That is, the protein as described herein can be used, for example, in combination with another protein for such use as described above.

The other protein is not particularly limited, so long as the effect provided by the protein as described herein is not degraded. The other protein may be, for example, a protein that improves cultivation of a cell, such as a stem cell, when using the other protein in combination with the protein as described herein as compared with when using the protein as described herein solely. Examples of improvement in cultivation of a cell can include, for example, improvement in proliferation of a cell.

Examples of the other protein can include, for example, the proteins (1) to (8) shown below:

(1) a protein that stabilizes a component in a culture medium;

(2) a protein including an amino acid sequence that stabilizes a component in a culture medium;

(3) a protein having a cell adhesion ability (cell adhesion protein);

(4) a protein including an amino acid sequence having a cell adhesion ability;

(5) a protein that promotes proliferation of a cell;

(6) a protein including an amino acid sequence that promotes proliferation of a cell;

(7) a protein that binds to any one of the proteins (1) to (6);

(8) a protein including an amino acid sequence that binds to any one of the proteins (1) to (6).

Examples of the protein (1) can include, for example, heparin. Heparin has been known to stabilize a basic fibroblast growth factor (bFGF) in a culture medium. That is, examples of the component in a culture medium can include, for example, bFGF. Examples of the protein (3) or (5) can include, for example, proteins having a scaffold function for a cell, such as fibronectin, vitronectin, collagen, osteopontin, and laminin. Examples of the protein (8) can include, for example, vitronectin. Vitronectin has been known to have a heparin-binding sequence.

Examples of the protein (2), (4), (6), or (8) can include, for example, fibroin-like proteins and proteins having a scaffold function for a cell, provided that the proteins have been inserted with the amino acid sequence recited in (2), (4), (6), or (8), respectively. The aforementioned descriptions concerning insertion of the cell adhesion sequence in the protein as described herein can be similarly applied to insertion of such an amino acid sequence. Examples of the amino acid sequence recited in (2), (4), (6), or (8) can include, for example, a partial sequence of the protein (1), (3), (5), or (7), respectively, provided that the partial sequence has the corresponding function. Specific examples of the amino acid sequence recited in (8) can include, for example, GKKQRFRHRNRKG (SEQ ID NO: 34). The amino acid sequence of SEQ ID NO: 34 is an amino acid sequence having the heparin-binding sequence of vitronectin and G added at the both termini. Specific examples of proteins having a scaffold function for a cell can include, for example, proteins exemplified as the protein (3) or (5), such as fibronectin, vitronectin, collagen, osteopontin, and laminin. That is, the protein having a scaffold function for a cell per se may typically be the protein (3) or (5).

Specific examples of the other protein can include, for example, a protein having the amino acid sequence shown in SEQ ID NO: 50.

The other protein or a component thereof may also be a variant of any of the amino acid sequences exemplified above or other known amino acid sequences, so long as the intended effect is obtained. The aforementioned descriptions concerning conservative variants of the fibroin-like protein can be similarly applied to variants of the other protein or a component thereof.

<2> Composition of the Present Invention

The composition as described herein is a composition containing the protein as described herein. The intended use of the composition as described herein is not particularly limited. The composition as described herein may be used for, for example, medical use or research use. That is, the composition as described herein may be, for example, a composition for medical or research use. The composition as described herein may be used for, specifically, for example, culturing a cell, such as a stem cell. That is, the composition as described herein may be, specifically, for example, a composition for culturing a cell. The composition as described herein may be, more specifically, for example, a cell scaffold material. The term “cell scaffold material” can refer to a structure that functions as a scaffold for a cell, specifically, a structure that functions as a scaffold for a cell when culturing the cell. The cell scaffold material can be used for, for example, culturing a cell. That is, a cell can be cultured using the cell scaffold material as a scaffold. The product obtained by culturing a cell using the cell scaffold material, such as cultured cells, cell sheet, tissue, and organ, can be used for a desired use such as medical treatment, for example, after isolation from the cell scaffold material, or together with the cell scaffold material. The cell scaffold material may also be implanted into a living body, and thereby used for culturing a cell in vivo, such as tissue formation mediated by adhesion and proliferation of cells. The composition as described herein may also be, specifically, for example, a coating agent for an apparatus such as a cell culture vessel. The descriptions below concerning the apparatus as described herein and the method as described herein can be similarly applied to an apparatus and cell culture.

The composition as described herein may or may not include the protein as described herein. The composition as described herein may include a combination of the protein as described herein and another ingredient. That is, the protein as described herein can be used, solely or in combination with another ingredient, as the composition as described herein. The other ingredient is not particularly limited, so long as it has acceptable properties depending on the use mode of the composition as described herein. Examples of the other ingredient can include, for example, ingredients blended and used in pharmaceuticals or reagents. Specific examples of such ingredients can include, for example, additives such as an excipient, binder, disintegrant, lubricant, stabilizer, flavoring agent, odor improving agent, perfume, diluent, and surfactant. Examples of the other ingredient also can include, for example, another protein (i.e. a protein other than the protein as described herein). The other protein is as described above.

The concentration (contained amount) of the protein as described herein in the composition as described herein is not particularly limited. The concentration of the protein as described herein in the composition as described herein can be appropriately chosen according to various conditions such as use mode of the composition as described herein. The concentration of the protein as described herein in the composition as described herein, for example, may be 0.01% (w/w) or more, 0.1% (w/w) or more, 1% (w/w) or more, 5% (w/w) or more, or 10% (w/w) or more, and may be 100% (w/w) or less, 99.9% (w/w) or less, 70% (w/w) or less, 50% (w/w) or less, 30% (w/w) or less, 10% (w/w) or less, 5% (w/w) or less, or 1% (w/w) or less, or may be within a range defined as a non-contradictory combination thereof.

The form of the composition as described herein is not particularly limited. The form of the composition as described herein can be appropriately chosen according to various conditions such as use mode of the composition as described herein. The composition as described herein may be formulated in a desired form. Specific examples of the form of the composition as described herein can include, for example, liquid (such as solution or suspension), paste, powder, granule, sphere, tablet, foam, sponge, fiber, film, sheet, membrane, mesh, and tube.

The composition as described herein may be used for a desired use such as coating of an apparatus, for example, as it is, or after being diluted, dispersed, dissolved, or the like in a medium such as water or an aqueous buffer as required. Needless to say, the product obtained by such dilution, dispersion, dissolution, or the like as described above can still be included in the scope of the composition as described herein.

<3> Apparatus of the Present Invention

The apparatus as described herein is an apparatus that can include the protein as described herein. The intended use of the apparatus as described herein is not particularly limited. The apparatus as described herein may be used for, for example, medical use or research use. That is, the apparatus as described herein may be, for example, an apparatus for medical or research use. The apparatus as described herein may be used for, specifically, for example, culturing a cell, such as a stem cell. That is, the apparatus as described herein may be, specifically, for example, an apparatus for culturing a cell. Examples of the apparatus for culturing a cell can include, for example, a cell culture vessel and a cell scaffold material. The apparatus as described herein as a cell culture vessel can also be referred to as “cell culture vessel as described herein”. The apparatus as described herein as a cell scaffold material can also be referred to as “cell scaffold material as described herein”. It is sufficient that the apparatus as described herein is an apparatus that includes the protein as described herein in such a manner that the function of the protein as described herein can be exercised. The apparatus as described herein may include the protein as described herein on the surface thereof. That is, the apparatus as described herein may be, specifically, for example, an apparatus of which the surface is coated with the protein as described herein. In addition, when the protein as described herein or the composition as described herein is configured as a form usable as an apparatus as it is, the protein as described herein or the composition as described herein may be used as the apparatus as described herein as it is. Specifically, for example, when the composition as described herein is configured as a cell scaffold material, the composition as described herein may be used as the cell scaffold material as described herein as it is. The descriptions below concerning the method as described herein can be similarly applied to cell culture.

The apparatus as described herein may further include another protein (i.e. a protein other than the protein as described herein). The other protein is as described above. The aforementioned descriptions concerning the use mode of the protein as described herein in the apparatus as described herein can be similarly applied to the use mode of the other protein in the apparatus as described herein. For example, the apparatus as described herein may be an apparatus of which the surface is coated with the other protein as well as the protein as described herein.

Hereinafter, although the apparatus as described herein is explained in reference mainly to a culture vessel, such explanations can be similarly applied to other apparatuses. That is, the explanations below can be applied to other apparatuses as it is, or after being modified as required according to various conditions such as the embodiment and intended use of the apparatuses.

The culture vessel is not particularly limited, so long as it is made of a material and has a form, which material and form are usable for culturing a cell. Specific examples of the form of the culture vessel can include, for example, a dish, multi-well plate, flask, cell insert, and membrane. Specific examples of the material of the culture vessel can include, for example, plastics such as polyethylene terephthalate (PET), polystyrene (PS), polycarbonate (PC), triacetyl cellulose (TAC), polyimide (PI), nylon (Ny), low-density polyethylene (LDPE), medium-density polyethylene (MDPE), vinyl chloride, vinylidene chloride, polyphenylene sulfide, polyether sulfone, polyethylene naphthalate, polypropylene, and urethane acrylate; biodegradable polymers such as polylactic acid, polyglycolic acid, polycaprolactone, and copolymers thereof glasses such as glass and modified glass; ceramics; metals; cellulose; and other various macromolecular compounds.

The culture vessel as described herein can include the protein as described herein at least on a cell culture surface thereof. That is, the culture vessel as described herein may have, for example, a cell culture surface coated with the protein as described herein. The term “cell culture surface” can refer to a surface on which a cell is cultured, specifically, a portion that can be contact with a cell upon culturing the cell. The cell culture surface of the culture vessel as described herein may or may not be wholly coated with the protein as described herein, so long as a cell, such as a stem cell, can be cultured using the culture vessel. The ratio of the area of the portion coated with the protein as described herein with respect to the total area of the cell culture surface of the culture vessel may be, for example, 50% or more, 70% or more, 80% or more, 90% or more, or 95% or more. The cell culture surface of the culture vessel can be wholly coated with the protein as described herein. For example, when the culture vessel is a dish-form vessel, the bottom surface can be wholly coated with the protein as described herein.

Methods for coating a cell culture surface of a culture vessel are not particularly limited. The cell culture surface of the culture vessel can be coated with the protein as described herein by, for example, known methods for coating a surface of a culture vessel with a functional material. For example, by applying a solution containing the protein as described herein to a cell culture surface of a culture vessel, the cell culture surface of the culture vessel can be coated with the protein as described herein. Specifically, for example, by applying a solution containing the protein as described herein to a cell culture surface of a culture vessel and drying the solution (i.e. volatilizing the solvent thereof), the cell culture surface of the culture vessel can be coated with the protein as described herein. Also, specifically, for example, by applying a solution containing the protein as described herein to a cell culture surface of a culture vessel in combination with a subsequent lapse of time (hereinafter, also referred to as “coating period”), the cell culture surface of the culture vessel can be coated with the protein as described herein. The coating period is not particularly limited, so long as the cell culture surface of the culture vessel can be coated with the protein as described herein to a desired extent. The coating period, for example, may be 30 minutes or longer, 1 hour or longer, 6 hours or longer, 12 hours or longer, or 18 hours or longer, may be 72 hours or shorter, 48 hours or shorter, or 30 hours or shorter, or may be within a range defined as a combination thereof. After the lapse of the coating period, the remaining solution may be removed or dried as required. The culture vessel may be sterilized as required. The culture vessel may be sterilized, for example, after the cell culture surface of the culture vessel is coated with the protein as described herein. For sterilization, for example, an alcohol such as ethanol and 2-propanol can be used. The alcohol may be used as, for example, an aqueous alcohol such as 70% ethanol. Sterilization can be carried out by, for example, applying an alcohol to the cell culture surface, or immersing the culture vessel in an alcohol. Although sterilization can be carried out by using an alcohol as described above, just in case, a solution containing the protein as described herein may be applied to the cell culture surface of the culture vessel after sterilization of the solution by filter sterilization or the like, and subsequence operation(s) may be aseptically carried out. For filter sterilization, for example, a filter such as Millex of Merck Millipore can be used. Operation(s) may be aseptically carried out as required. When a culture vessel is coated with both the protein as described herein and another protein, coating with the protein as described herein and coating with the other protein may be simultaneously carried out, or may be separately carried out. When coating with the protein as described herein and coating with the other protein are separately carried out, the order of them is not particularly limited. The culture vessel of the protein as described herein may be used immediately after production (i.e. immediately after coating with the protein as described herein), or after being stored as required. The culture vessel of the protein as described herein is effective for culturing a cell, such as a stem cell, even when the culture vessel is not used immediately after production. The temperature of storage may be, for example, 0 to 50° C., 0 to 40° C., 0 to 25° C., or 0 to 10° C. The period for storage may be, for example, 1 year or shorter, 6 months or shorter, or 1 month or shorter. The culture vessel of the protein as described herein may be stored in a dry state or a wet state. The culture vessel of the protein as described herein may be stored, for example, in a state where the cell culture surface is wet with a buffer such as PBS.

The coating amount of the protein as described herein to the surface of the culture vessel is not particularly limited, so long as a cell, such as a stem cell, can be cultured using the culture vessel as described herein. The coating amount of the protein as described herein to the surface of the culture vessel, for example, may be 0.1 μg/cm² or more, 0.2 μg/cm² or more, 0.4 μg/cm² or more, 1 μg/cm² or more, or 5 μg/cm² or more, and may be 500 μg/cm² or less, 250 μg/cm² or less, 100 μg/cm² or less, or 50 μg/cm² or less, or may be within a range defined as a combination thereof. The coating amount of the protein as described herein to the surface of the culture vessel may be, specifically, for example, 0.1 μg/cm² to 500 μg/cm², 0.2 μg/cm² to 250 μg/cm², or 0.4 μg/cm² to 100 μg/cm².

Similarly, the cell scaffold material as described herein can be appropriately obtained. The cell scaffold material is not particularly limited, so long as it is made of a material and has a form, which material and form are usable for culturing a cell. The cell scaffold material may have, for example, a form depending on the form of an objective resulting product obtained by culturing a cell, such as cell sheet, tissue, and organ. The cell scaffold material may be configured as a form having a cavity, such as a porous form, so that the inside of the cell scaffold material can be filled with cells. Examples of the material of the cell scaffold material can include, for example, bioabsorbable macromolecular compounds from the point of view that they are suitable for implant into a living body. Specific examples of bioabsorbable macromolecular compounds can include, for example, polylactic acid, polyglycolic acid, copolymer of lactic acid and glycolic acid, polycaprolactone, collagen, gelatin, glycosaminoglycan, chitin, chitosan, hyaluronic acid, and polypeptide. The cell scaffold material as described herein can be obtained by, for example, coating the surface (specifically, the cell culture surface) of the cell scaffold material with the protein as described herein. The term “surface of a cell scaffold material” used when the cell scaffold material is configured as a form having a cavity can include the surface of the cavity. Also, for example, as described above, the composition as described herein configured as a cell scaffold material may be used as the cell scaffold material as described herein as it is.

<4> Method of the Present Invention

A cell, such as a stem cell, can be efficiently cultured using the protein as described herein. Specifically, a cell, such as a stem cell, can be efficiently cultured using an apparatus comprising the protein as described herein (i.e. the apparatus as described herein). More specifically, a cell, such as a stem cell, can be efficiently cultured using a culture apparatus comprising the protein as described herein, such as the culture vessel as described herein and the cell scaffold material as described herein. That is, the present invention provides a method for culturing a cell, the method including culturing a cell using the apparatus as described herein, such as the culture vessel as described herein and the cell scaffold material as described herein. This method can also be referred to as “the method as described herein”. The protein as described herein is highly effective especially for proliferation of a stem cell such as an iPS cell. Hence, the protein as described herein and an apparatus for cell culture, such as a cell culture vessel and a cell scaffold material, including the same can be used for, for example, proliferation of a stem cell such as an iPS cell. Furthermore, the protein as described herein and an apparatus for cell culture, such as a cell culture vessel and a cell scaffold material, including the same can be used not only for proliferation of a stem cell such as an iPS cell, but also for, for example, establishment of a stem cell such as an iPS cell, which is a prior stage of proliferation of the stem cell, and differentiation of a stem cell such as an iPS cell, which is a posterior stage of proliferation of the stem cell. That is, the term “culturing a cell” can include not only proliferation of a cell, but also, for example, establishment of a cell having differentiation potency, such as a stem cell and a precursor cell, and differentiation from a cell having differentiation potency, such as a stem cell and a precursor cell, to another cell. Specifically, the term “culturing a stem cell” can include not only proliferation of a stem cell, but also, for example, establishment of a stem cell (e.g. establishment of a totipotent stem cell or a pluripotent stem cell, such as an iPS cell and an ES cell) and differentiation of a stem cell (e.g. differentiation from a stem cell, such as an iPS cell and an ES cell, to another stem cell, a precursor cell, or a mature cell). According to the method as described herein, cultured cells are obtained. That is, an embodiment of the method as described herein is a method for producing a cultured cell, the method including culturing a cell using the apparatus as described herein, such as the culture vessel as described herein and the cell scaffold material as described herein. Cultured cells obtained by the method as described herein may or may not be differentiated. That is, cultured cells obtained by the method as described herein, for example, may have been kept in undifferentiated state, or may have acquired an undifferentiated state. Cultured cells obtained by the method as described herein may each be, for example, a cell having differentiation potency, such as a stem cell and a precursor cell, or may be a differentiated cell, such as a precursor cell or a mature cell. Cultured cells obtained by the method as described herein, for example, may be free cells (i.e. individual independent cells), or may be organized cells, such as a cell sheet, tissue, and organ.

The type of cell to be cultured is not particularly limited. The method as described herein can be suitably used especially for culturing a stem cell. Specific examples of the stem cell can include, for example, an embryonic stem cell (ES cell), an induced pluripotent stem cell (iPS cell), a pluripotent germ stem cell (mGS cell), a neural stem cell, a mesenchymal stem cell, and a hematopoietic stem cell. More specific examples of the stem cell can include, for example, an embryonic stem cell (ES cell) and an induced pluripotent stem cell (iPS cell). The cell may be derived from any biological tissue. Examples of the biological tissue can include skin, cornea, liver, digestive organ, mammary gland, prostate, hair root, trachea, and oral mucosa. The organism from which the cell is derived is not particularly limited, and may be appropriately chosen depending on the use purpose or the like. Examples of the organism from which the cell is derived can include, for example, a mammal. Specific examples of the mammal can include, for example, human, rat, mouse, guinea pig, marmoset, rabbit, dog, cat, sheep, pig, and chimpanzee. The mammal may be immunodeficient. When using cultured cells for medical treatment of human, for example, a mammal such as a human, pig, or chimpanzee can be used for the organism from which the cell is derived. In the method as described herein, a single kind of cell may be cultured, or two or more kinds of cells may be co-cultured.

Culture conditions are not particularly limited, so long as an intended culture result (e.g. proliferation of a cell, establishment of a stem cell, and differentiation of a cell) is obtained. Culture conditions may be identical to, for example, those usually used for culturing a cell, such as an iPS cell and an ES cell, except that the apparatus as described herein, such as the culture vessel as described herein and the cell scaffold material as described herein, is used. Culture conditions can be appropriately chosen depending on various conditions such as the embodiment of the apparatus as described herein, the type of the cell, and the type of the organism from which the cell is derived.

The composition for the culture medium can be appropriately chosen depending on various conditions such as the properties of the cell to be cultured. Examples of culture medium components can include, for example, carbon sources such as glucose, amino acids, vitamins, phosphate salts, buffering agents, growth factors, serum, and serum albumin. Specific examples of usual culture media used for culturing a cell can include, for example, α-MEM, DMEM, and KCM. In addition, specific examples of usual culture media used for culturing an embryonic stem cell (ES cell) or an induced pluripotent stem cell (iPS cell) can include, for example, Essential 8 (Invitrogen), StemFit(R) medium (Ajinomoto), and other culture media used in the Examples section.

According to the method as described herein, a cell can be efficiently cultured without using a feeder cell or another cell adhesion protein (i.e. a cell adhesion protein other than the protein as described herein). However, this fact does not exclude use of a feeder cell or another cell adhesion protein. That is, in the method as described herein, a feeder cell or another cell adhesion protein (i.e. a cell adhesion protein other than the protein as described herein) may or may not be used. In the method as described herein, the amount of feeder cells or other cell adhesion proteins can be reduced as compared with the amount usually used to culture a cell. In the method as described herein, a feeder cell or another cell adhesion protein does not have to be used.

The number of cells inoculated at the start of culture, for example, may be 1×10³ cells/cm² or more, 1×10⁴ cells/cm² or more, or 2×10⁴ cells/cm² or more, and may be 1×10⁶ cells/cm² or less, 1×10⁵ cells/cm² or less, or 5×10⁴ cells/cm² or less, or may be within a range defined as a combination thereof. The culture temperature may be, for example, 36 to 38° C., or about 37° C. The culture pH may be, for example, pH6.8 to 7.2. The gas phase during culture may be, for example, a mixed gas of air and carbon dioxide. Specifically, for example, culture may be carried out under an atmosphere of 5% of carbon dioxide gas and 95% of air. The culture period may be, for example, 5 to 30 days, or 7 to 16 days. The culture medium may be exchanged during the culture.

In the method as described herein, the protein as described herein can be used in combination with another protein (i.e. a protein other than the protein as described herein). The other protein is as described above. For example, by using an apparatus including the protein as described herein (i.e. the apparatus as described herein), which apparatus further includes the other protein, the protein as described herein can be used in combination with the other protein. Also, for example, by using a culture medium containing the other protein, the protein as described herein can be used in combination with the other protein.

By carrying out cell culture as described above, cultured cells can be obtained.

### EXAMPLES

Hereinafter, the present invention will be more specifically explained with reference to the following non-limiting examples.

### Example 1

### Preparation of Cell Adhesion Sequence-Inserted Fibroin-Like Protein (1)

<1> Construction of Expression Strain for Cell Adhesion Sequence-Inserted Fibroin-Like Protein

A plasmid pET22b-ADF3 for expressing a gene encoding a fibroin-like protein ADF3 was constructed by digesting pET22b(+) vector (Novagen) with restriction enzymes NdeI and EcoRI, and inserting thereto a totally-synthesized fibroin-like protein gene ADF3 using DNA Ligation Kit (TaKaRa). The nucleotide sequence of the ADF3 gene is shown in SEQ ID NO: 1, and the amino acid sequence of the fibroin-like protein ADF3 encoded by this gene is shown in SEQ ID NO: 2. The ADF3 protein shown in SEQ ID NO: 2 has an His-tag and an HRV3C protease recognition sequence at the N-terminus.

A plasmid pET22b-ADF3RGDS#2 for expressing a gene encoding a cell adhesion sequence-inserted fibroin-like protein ADF3RGDS#2 was constructed by digesting pET22b(+) vector (Novagen) with restriction enzymes NdeI and EcoRI, and inserting thereto a totally-synthesized fibroin-like protein gene ADF3RGDS#2 using DNA Ligation Kit (TaKaRa). The nucleotide sequence of the ADF3RGDS#2 gene is shown in SEQ ID NO: 3, and the amino acid sequence of the fibroin-like protein ADF3RGDS#2 encoded by this gene is shown in SEQ ID NO: 4. The nucleotide sequence of positions 8 to 2200 of SEQ ID NO: 3 encodes the amino acid sequence shown in SEQ ID NO: 4. ADF3RGDS#2 consists of the ADF3 protein shown in SEQ ID NO: 2 except that 7 of 12 Ala-non-rich regions therein were each inserted with one unit (i.e. 7 units in total) of a cell adhesion sequence “GRGDSP” (SEQ ID NO: 22).

A plasmid pET22b-ADF3RGDS#3 for expressing a gene encoding a cell adhesion sequence-inserted fibroin-like protein ADF3RGDS#3 was constructed by digesting pET22b(+) vector (Novagen) with restriction enzymes NdeI and EcoRI, and inserting thereto a totally-synthesized fibroin-like protein gene ADF3RGDS#3 using DNA Ligation Kit (TaKaRa). The nucleotide sequence of the ADF3RGDS#3 gene is shown in SEQ ID NO: 5, and the amino acid sequence of the fibroin-like protein ADF3RGDS#3 encoded by this gene is shown in SEQ ID NO: 6. The nucleotide sequence of positions 8 to 2116 of SEQ ID NO: 5 encodes the amino acid sequence shown in SEQ ID NO: 6. ADF3RGDS#3 consists of the ADF3 protein shown in SEQ ID NO: 2 except that 7 of 12 Ala-non-rich regions therein were each inserted with one unit (i.e. 7 units in total) of a cell adhesion sequence “VTGRGDSPAS” (SEQ ID NO: 23).

Escherichia coli BLR(DE3) (F⁻ ompT hsdS_{B} (r_{B}⁻ m_{B}⁻ ) gal dcm (DE3) Δ(srl-recA) 306::Tn10 (Tet^{R})) was transformed with pET22b-ADF3, pET22b-ADF3RGDS#2, and pET22b-ADF3RGDS#3, to obtain fibroin-like protein-producing strains BLR(DE3)/pET22b-ADF3, BLR(DE3)/pET22b-ADF3RGDS#2, and BLR(DE3)/pET22b-ADF3RGDS#3, respectively.

<2> Expression of Cell Adhesion Sequence-Inserted Fibroin-Like Protein

The fibroin-like protein-expression strains BLR(DE3)/pET22b-ADF3, BLR(DE3)/pET22b-ADF3RGDS#2, and BLR(DE3)/pET22b-ADF3RGDS#3 obtained above were each cultured in a seed medium shown in (1) under conditions of lvvm, 37° C., 1500 rpm, and pH6.7 until glucose was completely consumed, to obtain a seed culture broth. Then, 45 mL of the seed culture broth was added to 255 mL of a production medium shown in (2), and main culture was carried out under conditions of 1 vvm, 37° C., 700 rpm, and pH6.9. At 30 minutes after the start of the culture, a feed medium shown in (3) was added at a feeding rate of 2.6 mL/h, and the culture was continued until the OD value of a culture broth at 620 nm reached 50. Then, the culture temperature was decreased to 30° C., and isopropyl-β-thiogalactopyranoside was added to a concentration of 1 mM to induce protein expression. The culture was finished at 24 hours after addition of IPTG, to obtain a culture broth. A disrupted cell suspension was prepared and applied to SDS-PAGE. As a result, a band having an objective size was observed, and that is, it was confirmed that the fibroin-like protein was expressed in cells.

(1) Seed Medium

Glucose 40 g/L, Magnesium sulfate heptahydrate 1.0 g/L, Corn steep liquor 1.0 g/L (in terms of nitrogen weight), Potassium dihydrogenphosphate 2.0 g/L, Iron sulfate heptahydrate 10 mg/L, Manganese sulfate heptahydrate 10 mg/L, Isoleucine 1.0 g/L, GD-113 (anti-foam agent) 0.1 ml/L, and Ampicillin 100 mg/L

(2) Production Medium

Glucose 2.5 g/L, Magnesium sulfate heptahydrate 2.4 g/L, Corn steep liquor 1.0 g/L (in terms of nitrogen weight), Potassium dihydrogenphosphate 9.0 g/L, Iron sulfate heptahydrate 40 mg/L, Manganese sulfate heptahydrate 40 mg/L, Calcium chloride dihydrate 40 mg/L, Isoleucine 3.0 g/L, GD-113 (anti-foam agent) 0.1 ml/L, and Ampicillin 100 mg/L

Glucose 700 g/L and Ampicillin 100 mg/L

<3> Purification of cell adhesion sequence-inserted fibroin-like protein

The obtained culture broth was diluted with a Lysis buffer shown in (1) so as to adjust the OD value to 30, and subjected to ultrasonic treatment. The treated product was centrifuged, and only the precipitate was collected. A 3% SDS solution was added to the precipitate in a volume equivalent to that of the treated product before centrifugation, and suspended for 30 min with a homogenizer. The suspension was centrifuged, and only the precipitate was collected again. The solubilization buffer shown in (2) was added to the precipitate in a volume equivalent to that of the treated product before centrifugation, and suspended for 3 hr with a homogenizer. The suspension was centrifuged, and only the supernatant was collected.

The obtained supernatant was mixed with cOmplete His-Tag Purification Resin (Roche) and stirred for 1 hr so as to allow proteins to bind thereto. The flow-through fraction was collected, contaminated proteins were removed with a washing buffer shown in (3) in a volume 20 times the column volume, and the objective protein was eluted with an elution buffer shown in (4) in a volume 10 times the column volume. The elution fraction was confirmed by SDS-PAGE, and concentrated with a flat sheet membrane of 10K.

The concentrate was diluted with the solubilization buffer shown in (2) so as to adjust the concentration to 0.04-0.05 mg/ml, to obtain 100 ml of the diluted solution. Dialysis was carried out with a dialysis membrane of 15K using 5 L of ultrapure water as the external liquid, to obtain aqueous solutions of the fibroin-like proteins ADF3, ADF3RGDS#2, and ADF3RGDS#3.

(1) Lysis Buffer

30 mM Tris-HCl and 100 mM NaCl, pH7.5

(2) Solubilization Buffer

8 M Urea, 20 mM NaH₂PO₄, and 0.5 M NaCl, pH8.0

(3) Washing Buffer

8 M Urea, 20 mM NaH₂PO₄, 0.5M NaCl, and 40 mM imidazole, pH8.0

(4) Elution Buffer

8 M Urea, 20 mM NaH₂PO₄, 0.5M NaCl, and 500 mM imidazole, pH8.0

### Example 2

### Preparation of Cell Adhesion Sequence-Inserted Fibroin-Like Protein (2)

<1> Construction of Expression Strain for Cell Adhesion Sequence-Inserted Fibroin-Like Protein

A plasmid pET22b-ADF3RGDS#53 for expressing a gene encoding a cell adhesion sequence-inserted fibroin-like protein ADF3RGDS#53 was constructed by digesting pET22b(+) vector (Novagen) with restriction enzymes NdeI and EcoRT, and inserting thereto a totally-synthesized fibroin-like protein gene ADF3RGDS#53 using DNA Ligation Kit (TaKaRa). The nucleotide sequence of the ADF3RGDS#53 gene is shown in SEQ ID NO: 26, and the amino acid sequence of the fibroin-like protein ADF3RGDS#53 encoded by this gene is shown in SEQ ID NO: 27. ADF3RGDS#53 consists of the ADF3 protein shown in SEQ ID NO: 2 except that the HRV3C protease recognition sequence therein was removed and 7 of 12 Ala-non-rich regions therein were each inserted with one unit (i.e. 7 units in total) of a cell adhesion sequence “PQVTRGDVFTM” (SEQ ID NO: 24).

A plasmid pET22b-ADF3RGDS#54 for expressing a gene encoding a cell adhesion sequence-inserted fibroin-like protein ADF3RGDS#54 was constructed by digesting pET22b(+) vector (Novagen) with restriction enzymes NdeI and EcoRI, and inserting thereto a totally-synthesized fibroin-like protein gene ADF3RGDS#54 using DNA Ligation Kit (TaKaRa). The nucleotide sequence of the ADF3RGDS#54 gene is shown in SEQ ID NO: 28, and the amino acid sequence of the fibroin-like protein ADF3RGDS#54 encoded by this gene is shown in SEQ ID NO: 29. ADF3RGDS#54 consists of the ADF3 protein shown in SEQ ID NO: 2 except that the HRV3C protease recognition sequence therein was removed and 7 of 12 Ala-non-rich regions therein were each inserted with one unit (i.e. 7 units in total) of a cell adhesion sequence “VTRGDVF” (SEQ ID NO: 25).

Escherichia coli BL21(DE3)ΔrecA was transformed with pET22b-ADF3RGDS#53 and pET22b-ADF3RGDS#54, to obtain fibroin-like protein-producing strains BL21(DE3)ΔrecA/pET22b-ADF3RGDS#53 and BL21(DE3)ΔrecA/pET22b-ADF3RGDS#54, respectively. The strain BL21(DE3)ΔrecA can be obtained from the strain BL21(DE3) by deletion of the recA gene using kRed method.

<2> Expression of Cell Adhesion Sequence-Inserted Fibroin-Like Protein

The fibroin-like protein-expression strains BL21ΔrecA/pET22b-ADF3RGDS#53 and BL21ΔrecA/pET22b-ADF3RGDS#54 obtained above were each cultured in a seed medium shown in (1) under conditions of lvvm, 37° C., 1500 rpm, and pH6.7 until glucose was completely consumed, to obtain a seed culture broth. Then, 45 mL of the seed culture broth was added to 255 mL of a production medium shown in (2), and main culture was carried out under conditions of lvvm, 37° C., 700rpm, and pH6.9. At 30 minutes after the start of the culture, a feed medium shown in (3) was added at a feeding rate of 2.6 mL/h, and the culture was continued until the OD value of a culture broth at 620 nm reached 50. Then, the culture temperature was decreased to 30° C., and isopropyl-β-thiogalactopyranoside (IPTG) was added at a concentration of 1 mM to induce protein expression. The culture was finished at 24 hours after addition of IPTG, to obtain a culture broth. A disrupted cell suspension was prepared and applied to SDS-PAGE. As a result, a band having an objective size was observed, and it was confirmed that the fibroin-like protein was expressed in the cells.

(1) Seed Medium

Glucose 40 g/L, Magnesium sulfate heptahydrate 1.0 g/L, Corn steep liquor 1.0 g/L (in terms of nitrogen weight), Potassium dihydrogenphosphate 2.0 g/L, Iron sulfate heptahydrate 10 mg/L, Manganese sulfate heptahydrate 10 mg/L, GD-113 (anti-foam agent) 0.1 ml/L, and Ampicillin 100 mg/L

(2) Production Medium

Glucose 2.5 g/L, Magnesium sulfate heptahydrate 2.4 g/L, Corn steep liquor 1.0 g/L (in terms of nitrogen weight), Potassium dihydrogenphosphate 5.4 g/L, Iron sulfate heptahydrate 40 mg/L, Manganese sulfate heptahydrate 40 mg/L, Calcium chloride dihydrate 40 mg/L, GD-113 (anti-foam agent) 0.1 ml/L, and Ampicillin 100 mg/L

(3) Feed Medium

Glucose 700 g/L and Ampicillin 100 mg/L

<3> Purification of Cell Adhesion Sequence-Inserted Fibroin-Like Protein

The obtained culture broth was diluted with a Lysis buffer shown in (1) so as to adjust the OD value to 30, and subjected to ultrasonic treatment. The treated product was centrifuged, and only the precipitate was collected. A 3% SDS solution was added to the precipitate in a volume equivalent to that of the treated product before centrifugation, and suspended for 30 min with a homogenizer. The suspension was centrifuged, and only the precipitate was collected again. The solubilization buffer shown in (2) was added to the precipitate so as to adjust the concentration of the objective protein to 5 mg/ml, and suspended with a Vortex mixer. The suspension was centrifuged, and only the supernatant was collected.

Dialysis was carried out for the obtained supernatant with a dialysis membrane of 500-1000 Da using 100 mL of an NaOH aqueous solution (pH10) as the external liquid, to obtain aqueous solutions of the fibroin-like proteins ADF3RGDS#53 and ADF3RGDS#54.

(1) Lysis Buffer

30 mM Tris-HCl and 100 mM NaCl, pH7.5

(2) Solubilization Buffer

50 mM NaOH

### Example 3

### Preparation of Cell Adhesion Sequence-Inserted Fibroin-Like Protein (3)

<1> Construction of Expression Strain for Cell Adhesion Sequence-Inserted Fibroin-Like Protein

A plasmid pET22b-ADF3RGDS#60 for expressing a gene encoding a cell adhesion sequence-inserted fibroin-like protein ADF3RGDS#60 was constructed by digesting pET22b(+) vector (Novagen) with restriction enzymes NdeI and EcoRI, and inserting thereto a totally-synthesized fibroin-like protein gene ADF3RGDS#60 using DNA Ligation Kit (TaKaRa). The nucleotide sequence of the ADF3RGDS#60 gene is shown in SEQ ID NO: 35, and the amino acid sequence of the fibroin-like protein ADF3RGDS#60 encoded by this gene is shown in SEQ ID NO: 36. ADF3RGDS#60 consists of the ADF3 protein shown in SEQ ID NO: 2 except that 7 of 12 Ala-non-rich regions therein were each inserted with one unit (i.e. 7 units in total) of a cell adhesion sequence “GRGDNP” (SEQ ID NO: 30).

A plasmid pET22b-ADF3RGDS#65 for expressing a gene encoding a cell adhesion sequence-inserted fibroin-like protein ADF3RGDS#65 was constructed by digesting pET22b(+) vector (Novagen) with restriction enzymes NdeI and EcoRI, and inserting thereto a totally-synthesized fibroin-like protein gene ADF3RGDS#65 using DNA Ligation Kit (TaKaRa). The nucleotide sequence of the ADF3RGDS#65 gene is shown in SEQ ID NO: 37, and the amino acid sequence of the fibroin-like protein ADF3RGDS#65 encoded by this gene is shown in SEQ ID NO: 38. ADF3RGDS#65 consists of the ADF3 protein shown in SEQ ID NO: 2 except that the HRV3C protease recognition sequence therein was removed and partial sequences of 7 of 12 Ala-non-rich regions therein were each replaced with a cell adhesion sequence “GAAGRGDSPAAGY” (SEQ ID NO: 31). In other words, it can be considered that the cell adhesion sequence has been inserted into the Ala-non-rich region in ADF3RGDS#65, which Ala-non-rich region is shorter than that of the ADF3 protein shown in SEQ ID NO: 2.

A plasmid pET22b-ADF3RGDS#68 for expressing a gene encoding a cell adhesion sequence-inserted fibroin-like protein ADF3RGDS#68 was constructed by digesting pET22b(+) vector (Novagen) with restriction enzymes NdeI and EcoRI, and inserting thereto a totally-synthesized fibroin-like protein gene ADF3RGDS#68 using DNA Ligation Kit (TaKaRa). The nucleotide sequence of the ADF3RGDS#68 gene is shown in SEQ ID NO: 39, and the amino acid sequence of the fibroin-like protein ADF3RGDS#68 encoded by this gene is shown in SEQ ID NO: 40. ADF3RGDS#68 consists of the ADF3 protein shown in SEQ ID NO: 2 except that the HRV3C protease recognition sequence therein was removed, 5 of 12 Ala-non-rich regions therein were each inserted with one unit (i.e. 5 units in total) of an amino acid sequence “GPGQQGPGQQGPGQQVTGRGDSPAS” (SEQ ID NO: 32) containing a cell adhesion sequence “VTGRGDSPAS” (SEQ ID NO: 23), and 2 of 12 Ala-non-rich regions therein were each inserted with one unit (i.e. 2 units in total) of a cell adhesion sequence “VTGRGDSPAS” (SEQ ID NO: 23). In other words, regarding the former 5 units in total of the cell adhesion sequence, it can be considered that the cell adhesion sequence has been inserted into the Ala-non-rich region in ADF3RGDS#68, which Ala-non-rich region is longer than that of the ADF3 protein shown in SEQ ID NO: 2.

A plasmid pET22b-ADF3RGDS#72 for expressing a gene encoding a cell adhesion sequence-inserted fibroin-like protein ADF3RGDS#72 was constructed by digesting pET22b(+) vector (Novagen) with restriction enzymes NdeI and EcoRI, and inserting thereto a totally-synthesized fibroin-like protein gene ADF3RGDS#72 using DNA Ligation Kit (TaKaRa). The nucleotide sequence of the ADF3RGDS#72 gene is shown in SEQ ID NO: 41, and the amino acid sequence of the fibroin-like protein ADF3RGDS#72 encoded by this gene is shown in SEQ ID NO: 42. ADF3RGDS#72 consists of the ADF3 protein shown in SEQ ID NO: 2 except that 12 Ala-non-rich regions therein were each inserted with one unit (i.e. 12 units in total) of a cell adhesion sequence “GRGDSP” (SEQ ID NO: 22).

A plasmid pET22b-ADF3RGDS#73 for expressing a gene encoding a cell adhesion sequence-inserted fibroin-like protein ADF3RGDS#73 was constructed by digesting pET22b(+) vector (Novagen) with restriction enzymes NdeI and EcoRI, and inserting thereto a totally-synthesized fibroin-like protein gene ADF3RGDS#73 using DNA Ligation Kit (TaKaRa). The nucleotide sequence of the ADF3RGDS#73 gene is shown in SEQ ID NO: 43, and the amino acid sequence of the fibroin-like protein ADF3RGDS#73 encoded by this gene is shown in SEQ ID NO: 44. ADF3RGDS#73 consists of the ADF3 protein shown in SEQ ID NO: 2 except that the HRV3C protease recognition sequence therein was removed and 2 of 12 Ala-non-rich regions therein were each inserted with one unit (i.e. 2 units in total) of a cell adhesion sequence “GRGDSP” (SEQ ID NO: 22).

A plasmid pET22b-ADF3RGDS#76 for expressing a gene encoding a cell adhesion sequence-inserted fibroin-like protein ADF3RGDS#76 was constructed by digesting pET22b(+) vector (Novagen) with restriction enzymes NdeI and EcoRI, and inserting thereto a totally-synthesized fibroin-like protein gene ADF3RGDS#76 using DNA Ligation Kit (TaKaRa). The nucleotide sequence of the ADF3RGDS#76 gene is shown in SEQ ID NO: 45, and the amino acid sequence of the fibroin-like protein ADF3RGDS#76 encoded by this gene is shown in SEQ ID NO: 46. ADF3RGDS#76 consists of the ADF3 protein shown in SEQ ID NO: 2 except that the HRV3C protease recognition sequence therein was removed and 7 of 12 Ala-non-rich regions therein were each inserted with one unit (i.e. 7 units in total) of an amino acid sequence “VTGRGDSPASVTGRGDSPAS” (SEQ ID NO: 33) consisting of two mutually-linked units of a cell adhesion sequence “VTGRGDSPAS” (SEQ ID NO: 23). In other words, it can be considered that 14 units in total of the cell adhesion sequence have been inserted in ADF3RGDS#76.

A plasmid pET22b-ADF3RGDS#77 for expressing a gene encoding a cell adhesion sequence-inserted fibroin-like protein ADF3RGDS#77 was constructed by digesting pET22b(+) vector (Novagen) with restriction enzymes NdeI and EcoRI, and inserting thereto a totally-synthesized fibroin-like protein gene ADF3RGDS#77 using DNA Ligation Kit (TaKaRa). The nucleotide sequence of the ADF3RGDS#77 gene is shown in SEQ ID NO: 47, and the amino acid sequence of the fibroin-like protein ADF3RGDS#77 encoded by this gene is shown in SEQ ID NO: 48. ADF3RGDS#77 consists of the ADF3 protein shown in SEQ ID NO: 2 except that 12 Ala-non-rich regions therein were each inserted with one unit (i.e. 12 units in total) of a cell adhesion sequence “VTGRGDSPAS” (SEQ ID NO: 23).

A plasmid pET22b-ADF3RGDS#141 for expressing a gene encoding a heparin-binding sequence-inserted fibroin-like protein ADF3RGDS#141 was constructed by digesting pET22b(+) vector (Novagen) with restriction enzymes NdeI and EcoRI, and inserting thereto a totally-synthesized fibroin-like protein gene ADF3RGDS#141 using DNA Ligation Kit (TaKaRa). The nucleotide sequence of the ADF3RGDS#141 gene is shown in SEQ ID NO: 49, and the amino acid sequence of the fibroin-like protein ADF3RGDS#141 encoded by this gene is shown in SEQ ID NO: 50. ADF3RGDS#141 consists of the ADF3 protein shown in SEQ ID NO: 2 except that 7 of 12 Ala-non-rich regions therein were each inserted with one unit (i.e. 7 units in total) of a heparin-binding sequence “GKKQRFRHRNRKG” (SEQ ID NO: 34). This heparin-binding sequence is an amino acid sequence consisting of the heparin-binding sequence of vitronectin (Wrighton P J, Klim J R, Hernandez B A, Koonce C H, Kamp T J, Kiessling L L, Proceedings of the National Academy of Sciences of the United States of America 2014 December; 111(51): 18126-18131., Signals from the surface modulate differentiation of human pluripotent stem cells through glycosaminoglycans and integrins.) added with G at the both termini.

A plasmid pET22b-ADF3RGDS#66 for expressing a gene encoding a cell adhesion sequence-inserted fibroin-like protein ADF3RGDS#66 was constructed by digesting

Atty Docket No: US-586 pET22b(+) vector (Novagen) with restriction enzymes NdeI and EcoRI, and inserting thereto a totally-synthesized fibroin-like protein gene ADF3RGDS#66 using DNA Ligation Kit (TaKaRa). The nucleotide sequence of the ADF3RGDS#66 gene is shown in SEQ ID NO: 51, and the amino acid sequence of the fibroin-like protein ADF3RGDS#66 encoded by this gene is shown in SEQ ID NO: 52. ADF3RGDS#66 consists of the ADF3 protein shown in SEQ ID NO: 2 except that the HRV3C protease recognition sequence therein was removed, partial sequences of 5 of 12 Ala-non-rich regions therein were each replaced with a cell adhesion sequence “VTGRGDSPAS” (SEQ ID NO: 23), and 2 of 12 Ala-non-rich regions therein were each inserted with one unit (i.e. 2 units in total) of a cell adhesion sequence “VTGRGDSPAS” (SEQ ID NO: 23). In other words, regarding the former 5 units in total of the cell adhesion sequence, it can be considered that the cell adhesion sequence has been inserted into the Ala-non-rich region in ADF3RGDS#66, which Ala-non-rich region is shorter than that of the ADF3 protein shown in SEQ ID NO: 2.

A plasmid pET22b-ADF3RGDS#78 for expressing a gene encoding a cell adhesion sequence-inserted fibroin-like protein ADF3RGDS#78 was constructed by digesting pET22b(+) vector (Novagen) with restriction enzymes NdeI and EcoRI, and inserting thereto a totally-synthesized fibroin-like protein gene ADF3RGDS#78 using DNA Ligation Kit (TaKaRa). The nucleotide sequence of the ADF3RGDS#78 gene is shown in SEQ ID NO: 53, and the amino acid sequence of the fibroin-like protein ADF3RGDS#78 encoded by this gene is shown in SEQ ID NO: 54. ADF3RGDS#78 consists of the ADF3 protein shown in SEQ ID NO: 2 except that the HRV3C protease recognition sequence therein was removed and 2 of 12 Ala-non-rich regions therein were each inserted with one unit (i.e. 2 units in total) of a cell adhesion sequence “VTGRGDSPAS” (SEQ ID NO: 23).

Escherichia coli BL21(DE3)ΔrecA was transformed with pET22b-ADF3RGDS#60, pET22b-ADF3RGDS#65, pET22b-ADF3RGDS#68, pET22b-ADF3RGDS#72, pET22b-ADF3RGDS#73, pET22b-ADF3RGDS#76, pET22b-ADF3RGDS#77, pET22b-ADF3RGDS#141, pET22b-ADF3RGDS#66, and pET22b-ADF3RGDS#78, to obtain fibroin-like protein-producing strains BL21(DE3)ΔrecA/pET22b-ADF3RGDS#60, BL21(DE3)ΔrecA/pET22b-ADF3RGDS#65, BL21(DE3)ΔrecA/pET22b-ADF3RGDS#68, BL21(DE3)ΔrecA/pET22b-ADF3RGDS#72, BL21(DE3)ΔrecA/pET22b-ADF3RGDS#73, BL21(DE3)ΔrecA/pET22b-ADF3RGDS#76, BL21(DE3)ΔrecA/pET22b-ADF3RGDS#77, BL21(DE3)ΔrecA/pET22b-ADF3RGDS#141, BL21(DE3)ΔrecA/pET22b-ADF3RGDS#66, and BL21(DE3)ΔrecA/pET22b-ADF3RGDS#78, respectively.

<2> Expression of cell adhesion sequence-inserted fibroin-like protein

The fibroin-like protein-expression strains BL21(DE3)ΔrecA/pET22b-ADF3RGDS#60, BL21(DE3)ΔrecA/pET22b-ADF3RGDS#65, BL21(DE3)ΔrecA/pET22b-ADF3RGDS#72, BL21(DE3)ΔrecA/pET22b-ADF3RGDS#73, and BL21(DE3)ΔrecA/pET22b-ADF3RGDS#141 obtained above were each cultured in a seed medium shown in (1) under conditions of lvvm, 37° C., 1500 rpm, and pH6.7 until glucose was completely consumed, to obtain a seed culture broth. Then, 45 mL of the seed culture broth was added to 255 mL of a production medium shown in (2), and main culture was carried out under conditions of lvvm, 37° C., 700 rpm, and pH6.9. At 30 minutes after the start of the culture, a feed medium shown in (3) was start to be added at a feeding rate of 2.6 mL/h, and the culture was continued until the OD value of a culture broth at 620 nm reached 50. Then, the culture temperature was decreased to 30° C., and isopropyl-β-thiogalactopyranoside (IPTG) was added at a concentration of 1 mM to induce protein expression. The culture was finished at 24 hours after addition of IPTG, to obtain a culture broth. A disrupted cell suspension was prepared and applied to SDS-PAGE. As a result, a band having an objective size was observed, and that is, it was confirmed that the fibroin-like protein was expressed in cells.

(1) Seed Medium

Glucose 40 g/L, Magnesium sulfate heptahydrate 1.0 g/L, Corn steep liquor 1.0 g/L (in terms of nitrogen weight), Potassium dihydrogenphosphate 2.0 g/L, Iron sulfate heptahydrate 10 mg/L, Manganese sulfate heptahydrate 10 mg/L, GD-113 (anti-foam agent) 0.1 ml/L, and Ampicillin 100 mg/L

(2) Production Medium

Glucose 2.5 g/L, Magnesium sulfate heptahydrate 2.4 g/L, Corn steep liquor 1.0 g/L (in terms of nitrogen weight), Potassium dihydrogenphosphate 5.4 g/L, Iron sulfate heptahydrate 40 mg/L, Manganese sulfate heptahydrate 40 mg/L, Calcium chloride dihydrate 40 mg/L, GD-113 (anti-foam agent) 0.1 ml/L, and Ampicillin 100 mg/L

(3) Feed Medium

Glucose 700 g/L and Ampicillin 100 mg/L

The fibroin-like protein-expression strains BL21(DE3)ΔrecA/pET22b-ADF3RGDS#68, BL21(DE3)ΔrecA/pET22b-ADF3RGDS#76, BL21(DE3)ΔrecA/pET22b-ADF3RGDS#77, BL21(DE3)ΔrecA/pET22b-ADF3RGDS#66, and BL21(DE3)ΔrecA/pET22b-ADF3RGDS#78 obtained above are each cultured in a seed medium shown in (1) under conditions of lvvm, 37° C., 1500 rpm, and pH6.7 until glucose is completely consumed, to obtain a seed culture broth. Then, 45 mL of the seed culture broth is added to 255 mL of a production medium shown in (2), and main culture is carried out under conditions of lvvm, 37° C., 700 rpm, and pH6.9. At 30 minutes after the start of the culture, a feed medium shown in (3) is added at a feeding rate of 2.6 mL/h, and the culture is continued until the OD value of a culture broth at 620 nm reached 50. Then, the culture temperature is decreased to 30° C., and isopropyl-β-thiogalactopyranoside (IPTG) is added at a concentration of 1 mM to induce protein expression. The culture is finished at 24 hours after addition of IPTG, to obtain a culture broth. Then, a disrupted cell suspension is prepared and applied to SDS-PAGE, to confirm the expression of the objective fibroin-like protein.

(1) Seed Medium

Glucose 40 g/L, Magnesium sulfate heptahydrate 1.0 g/L, Corn steep liquor 1.0 g/L (in terms of nitrogen weight), Potassium dihydrogenphosphate 2.0 g/L, Iron sulfate heptahydrate 10 mg/L, Manganese sulfate heptahydrate 10 mg/L, GD-113 (anti-foam agent) 0.1 ml/L, and Ampicillin 100 mg/L

(2) Production Medium

Glucose 2.5 g/L, Magnesium sulfate heptahydrate 2.4 g/L, Corn steep liquor 1.0 g/L (in terms of nitrogen weight), Potassium dihydrogenphosphate 5.4 g/L, Iron sulfate heptahydrate 40 mg/L, Manganese sulfate heptahydrate 40 mg/L, Calcium chloride dihydrate 40 mg/L, GD-113 (anti-foam agent) 0.1 ml/L, and Ampicillin 100 mg/L

(3) Feed Medium

Glucose 700 g/L and Ampicillin 100 mg/L

<3> Purification of Cell Adhesion Sequence-Inserted Fibroin-Like Protein

The obtained culture broth of each of the fibroin-like protein-expression strains BL21(DE3)ΔrecA/pET22b-ADF3RGDS#60, BL21(DE3)ΔrecA/pET22b-ADF3RGDS#65, BL21(DE3)ΔrecA/pET22b-ADF3RGDS#72, and BL21(DE3)ΔrecA/pET22b-ADF3RGDS#141 was diluted with a Lysis buffer shown in (1) so as to adjust the OD value to 30, and subjected to ultrasonic treatment. The treated product was centrifuged, and only the precipitate was collected. The precipitate was added with a 3% SDS solution in a volume equivalent to that of the treated product before centrifugation, and suspended for 5 min or longer with a homogenizer. The suspension was centrifuged, and only the precipitate was collected again. The precipitate was added with a solubilization buffer shown in (2) so as to adjust the concentration of the objective protein to 1-5 mg/ml, and suspended with a Vortex mixer. The suspension was centrifuged, and only the supernatant was collected.

Dialysis was carried out for 1 ml of the obtained supernatant with a dialysis membrane of 500-1000 Da using 100 mL of an NaOH aqueous solution (pH10) as the external liquid, to obtain aqueous solutions of the fibroin-like proteins ADF3RGDS#60, ADF3RGDS#65, ADF3RGDS#72, and ADF3RGDS#141.

(1) Lysis Buffer

30 mM Tris-HCl and 100 mM NaCl, pH7.5

(2) Solubilization Buffer

50 mM NaOH

The obtained culture broth of the fibroin-like protein-expression strain BL21(DE3)ΔrecA/pET22b-ADF3RGDS#73 was diluted with a Lysis buffer shown in (1) so as to adjust the OD value to 30, and subjected to ultrasonic treatment. The treated product was centrifuged, and only the precipitate was collected. The precipitate was added with a 3% SDS solution in a volume equivalent to that of the treated product before centrifugation, and suspended for 5 min or longer with a homogenizer. The suspension was centrifuged, and only the precipitate was collected again. The precipitate was added with a solubilization buffer shown in (2) in a volume equivalent to that of the treated product before centrifugation, and suspended for 1 hr with a homogenizer. The suspension was centrifuged, and only the supernatant was collected.

The obtained supernatant was mixed with cOmplete His-Tag Purification Resin (Roche) and stirred for 1 hr 30 min so as to allow proteins to bind thereto. The flow-through fraction was collected, contaminated proteins were removed with a washing buffer shown in (3) in a volume 20 times the column volume, and the objective fibroin-like protein was eluted with an elution buffer shown in (4) in a volume 10 times the column volume. The elution fraction was confirmed by SDS-PAGE, and concentrated with a membrane of 30 KDa.

The concentrate was subjected to buffer exchange with a solution shown in (5) using a membrane of 30 KDa, to be adjusted to a volume providing a concentration of 0.1 mg/ml. Dialysis was carried out for 1 ml of the solution after the buffer exchange with a dialysis membrane of 500-1000 Da using 100 mL of an NaOH aqueous solution (pH10) as the external liquid, to obtain an aqueous solution of the fibroin-like protein ADF3RGDS#73.

(1) Lysis Buffer

30 mM Tris-HCl and 100 mM NaCl, pH7.5

(2) Solubilization Buffer

M Urea, 20 mM NaH₂PO4, and 0.5 M NaCl, pH8.0

(3) Washing Buffer

8 M Urea, 20 mM NaH₂PO₄, 0.5M NaCl, and 40 mM imidazole, pH8.0

(4) Elution Buffer

8 M Urea, 20 mM NaH₂PO₄, 0.5M NaCl, and 500 mM imidazole, pH8.0

(5) Buffer Exchange Solution

50 mM NaOH

The obtained culture broth of each of the fibroin-like protein-expression strains BL21(DE3)ΔrecA/pET22b-ADF3RGDS#68, BL21(DE3)ΔrecA/pET22b-ADF3RGDS#76, BL21(DE3)ΔrecA/pET22b-ADF3RGDS#77, BL21(DE3)ΔrecA/pET22b-ADF3RGDS#66, and BL21(DE3)ΔrecA/pET22b-ADF3RGDS#78 is diluted with a Lysis buffer shown in (1) so as to adjust the OD value to 30, and subjected to ultrasonic treatment. The treated product is centrifuged, and only the precipitate is collected. A 3% SDS solution in a volume equivalent to that of the treated product before centrifugation is added to the precipitate, and suspended for 5 min or longer with a homogenizer. The suspension is centrifuged, and only the precipitate is collected again. The solubilization buffer shown in (2) is added to the precipitate so as to adjust the concentration of the objective protein to 1-5 mg/ml, and suspended with a Vortex mixer. The suspension is centrifuged, and only the supernatant is collected.

Dialysis is carried out for 1 ml of the obtained supernatant with a dialysis membrane of 500-1000 Da using 100 mL of an NaOH aqueous solution (pH10) as the external liquid, to obtain aqueous solutions of the fibroin-like proteins ADF3RGDS#68, ADF3RGDS#76, ADF3RGDS#77, ADF3RGDS#66, and ADF3RGDS#78.

(1) Lysis Buffer

30 mM Tris-HCl and 100 mM NaCl, pH7.5

(2) Solubilization Buffer

50 mM NaOH

### Example 4

### Evaluation of Cell Scaffold Function of Fibroin-Like Protein

<1> Coating to Cell Culture Plate

<1-1> Coating to Cell Culture Plate (1)

The aqueous solutions of fibroin-like proteins after dialysis obtained in Examples 1<3> and 3<3> were applied to a 6-well cell culture plate (Falcon cell culture plate; Corning) in volumes of 1 to 4 ml/well. The cell culture plate was left to stand at a room temperature for a day and night or for two days and nights, so that water was dried. The cell culture plate was immersed into a 70% ethanol aqueous solution, and then washed with phosphate buffered saline (Nacalai Tesque).

<1-2> Coating to Cell Culture Plate (2)

The aqueous solutions of fibroin-like proteins after dialysis obtained in Examples 2<3> and 3<3> were applied to a 6-well cell culture plate (Falcon cell culture plate; Corning) in volumes of 1 to 4 ml/well. The cell culture plate was left to stand at 4° C. for a day and night, and then the aqueous solutions were removed. The cell culture plate was immersed into a 70% ethanol aqueous solution, and then washed with phosphate buffered saline (Nacalai Tesque).

<2> Confirmation of Coating

The coating surface was analyzed by FT-IR attenuated total reflection (FT-IR-ATR) using IR Prestige-21 (Shimadzu Corporation) and an ATR measuring device DuraSamplJR II (Smiths). A peak derived from an amide group of a protein main chain (a-amide bond) was observed in the range of 1620 to 1700 cm⁻ in the obtained spectrum, and hence, it was confirmed that the fibroin-like protein was coated on the cell culture plate. The coating amount of the fibroin-like protein is considered to be approximately 10 μg/cm² when coating is carried out by the method of <1-1>.

<3> Evaluation in iPS Cell Proliferation System

The effect on adhesion and proliferation of an induced pluripotent stem cell (iPS cell) on the surface coated with the fibroin-like protein was evaluated. As the iPS cell, the strain 201B7 purchased from iPS Portal was used. Cell culture was carried out under conditions of 5% CO₂/37° C. using a culture medium obtained by adding human serum albumin (final concentration of 2.6 mg/ml; Sigma-Aldrich) to Essential 8 medium (Invitrogen), which is a culture medium for iPS cells, or using StemFit(R) medium (Ajinomoto). The cell culture plate was used for culture immediately after coating with the fibroin-like protein, or was stored for 6 days at 4° C. after coating with the fibroin-like protein and then used for culture, and thereby the effect of the coating was investigated. Viable cells were inoculated as single cells in amounts of 20,000 to 100,000 cells/well. The culture medium used at the time of the inoculation was added with Y-27632 (final concentration of 10 μM, Nacalai Tesque), and from the next day, culture was carried out with the culture medium not added with Y-27632. The culture medium was exchanged every 1 to 3 days, and culture was carried out for 6 to 7 days. Then, the number of cells released by a treatment with a cell dissociation enzyme (TrypLE Select; Life Technologies) was measured with a cell counter (ViCell; Beckman Coulter). As a comparative control, Pronectin (Sanyo Chemical Industries), which is an artificial cell adhesion peptide, was coated on the cell culture plate according to the product protocol, and evaluation was carried out with the same culture method. As further comparative controls, evaluation was also carried out for various cell culture vessels coated with a basement membrane matrix or a mimic thereof (BioCoat Matrigel of Corning, BioCoat Fibronectin of Corning, and PureCoat Fibronectin peptide of Corning) with the same culture method. Among them, when using BioCoat Matrigel (Corning), the first exchange of the culture medium (i.e. exchange from the culture medium containing Y-27632 used at the time of the inoculation to the culture medium not containing Y-27632) was carried out at 48 hours after the inoculation.

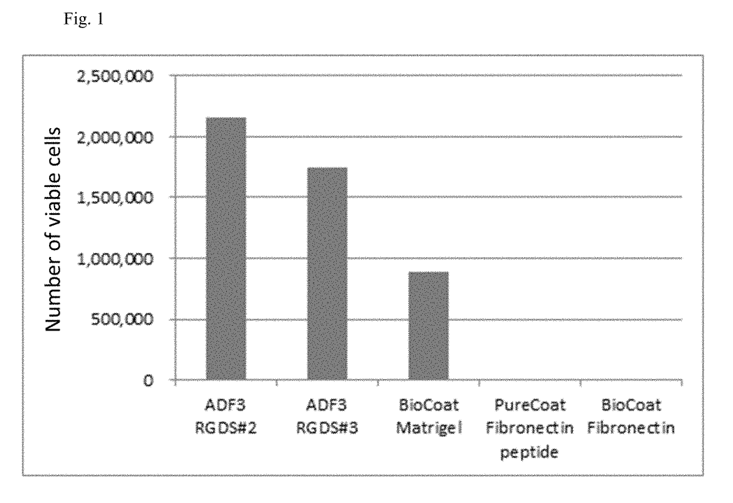

Results are shown in FIGS. 1-7 and Table 1.

As shown in FIG. 1, when any of ADF3RGDS#2 and ADF3RGDS#3, which are fibroin-like proteins inserted with a cell adhesion sequence, was used for coating with the method of <1-1> and 100,000 viable cells were inoculated, the number of viable cells after 6 days was larger than that observed with BioCoat Matrigel, which is commercially available as a culture vessel for pluripotent stem cells, and that is, it was revealed that ADF3RGDS#2 and ADF3RGDS#3 have a favorable scaffold function for an iPS cell. Although ADF3RGDS#2 and ADF3RGDS#3 each have been inserted with a cell adhesion sequence of fibronectin, the number of viable cells at 6 days after the inoculation was zero when using BioCoat Fibronectin, which is coated with fibronectin per se, or PureCoat Fibronectin peptide, which is coated with a mimic peptide of fibronectin.

When evaluation was carried out for ADF3, which is a fibroin-like protein not inserted with a cell adhesion sequence, with the same method, adhesion of inoculated cells was scarcely observed, and the number of viable cells at 6 days after the inoculation was zero (data not shown). This indicates that a scaffold function for an iPS cell was obtained due to insertion of a cell adhesion sequence.

When evaluation was carried out for Pronectin (Sanyo Chemical Industries), which is a fibroin of silkworm inserted with the same cell adhesion sequence as that inserted in ADF3RGDS#3, with the same method, the number of viable cells at 6 days after the inoculation was zero as with the case of ADF3 (data not shown).

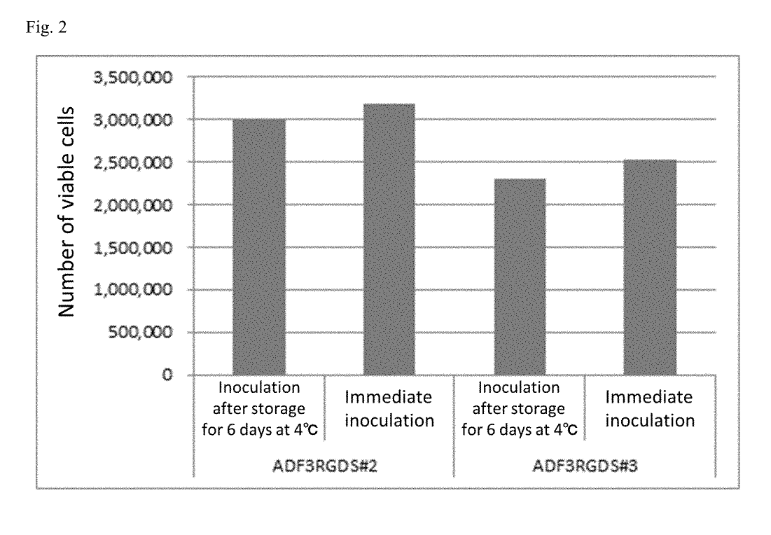

As shown in FIG. 2, even when any of ADF3RGDS#2 and ADF3RGDS#3, which are fibroin-like proteins inserted with a cell adhesion sequence, was coated on the cell culture plate with the method of <1-1> and the cell culture plate was used for cell culture after storage for 6 days at 4° C., the number of viable cells at 6 days after the inoculation of 100,000 viable cells was equivalent to that observed when the cell culture plate was used for cell culture immediately after the coating. Regarding basement membrane matrices (proteins) used for feeder-free culture of pluripotent stem cells, such as iMatrix (Nippi), it is recommended, according to the product protocol, that a cell culture plate is used for cell culture immediately after coating therewith. By contrast, it is not necessary that a cell culture plate is used for cell culture immediately after coating with the product as described herein, and hence, the product as described herein enables easily carrying out culture of pluripotent stem cells.

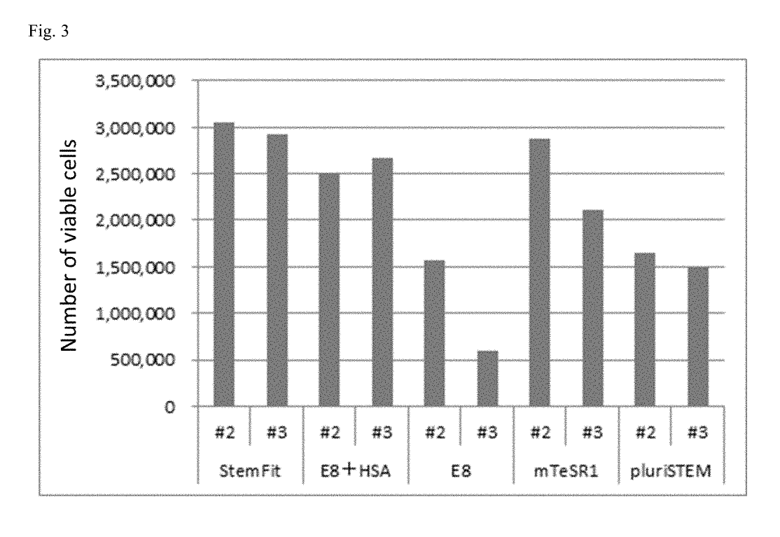

As shown in FIG. 3, when any of ADF3RGDS#2 (#2 in the figure) and ADF3RGDS#3 (#3 in the figure), which are fibroin-like proteins inserted with a cell adhesion sequence, was used for coating with the method of <1-1> and 100,000 viable cells were inoculated, they showed a favorable scaffold function for an iPS cell even when cell culture was carrying out using any of Essential 8 medium alone (E8 in the figure), mTeSR1 medium (STEMCELL Technologies), and pluriSTEM medium (Merck Millipore), as well as using StemFit(R) medium (Ajinomoto) or the culture medium (E8+HSA in the figure) obtained by adding human serum albumin to Essential 8 medium (Invitrogen). This indicates that the product as described herein is not limited to be used in a specific culture medium, and hence, it is highly versatile.

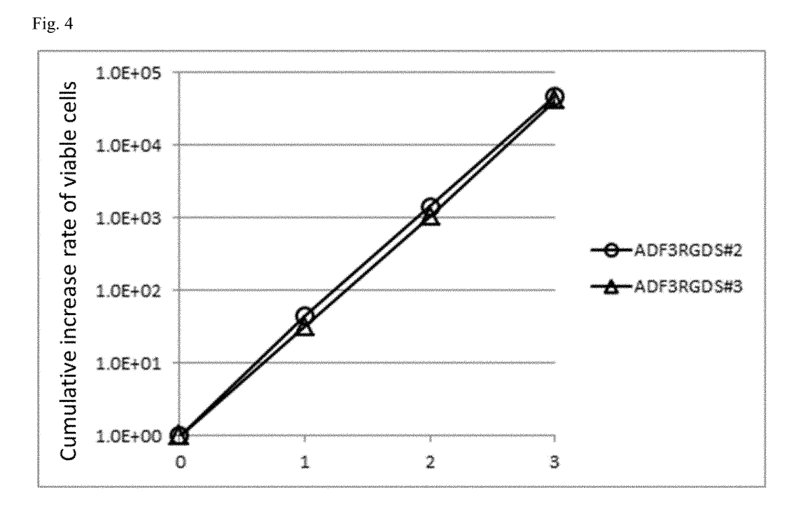

As shown in FIG. 4, when any of ADF3RGDS#2 and ADF3RGDS#3, which are fibroin-like proteins inserted with a cell adhesion sequence, was used for coating with the method of <1-1> and 100,000 viable cells were inoculated, culture was successfully carried out over a period of 3 weeks wherein subculture was carried out twice. The increase rate of viable cells was not changed during the culture, and that is, it was indicated that culture can be stably carried out. After 3 weeks of the culture, cells were stained with an alkaline phosphatase staining kit (86-R; Sigma-Aldrich), and as a result, it was confirmed that cells were kept in undifferentiated state. This indicates that the product as described herein enables an iPS cell to proliferate while maintaining undifferentiated state thereof in a long-term culture.

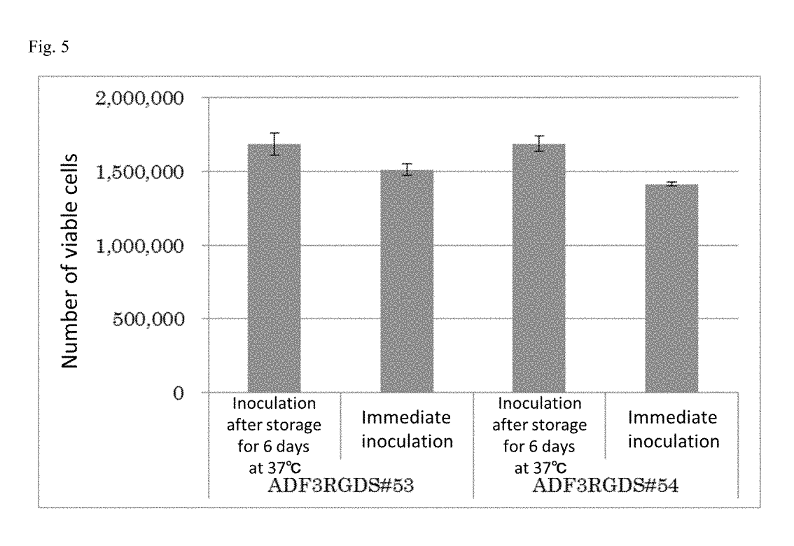

As shown in FIG. 5, even when any of ADF3RGDS#53 and ADF3RGDS#54, which are fibroin-like proteins inserted with a cell adhesion sequence, was coated on the cell culture plate with the method of <1-2> and the cell culture plate was used for cell culture after storage for 6 days at 37° C., the number of viable cells at 6 days after the inoculation of 20,000 viable cells was equivalent to that observed when the cell culture plate was used for cell culture immediately after the coating. That is, it was indicated that coating can be carried out with a method in a short time with a low contamination risk as compared with the method of <1-1>, the product as described herein can be stored at a normal temperature, and a pluripotent stem cell can be cultured by using the product as described herein without using a large amount of cells. This indicates that the product as described herein is widely superior as a scaffold material for an iPS cell in terms not only of functions but also of operability and stability etc.

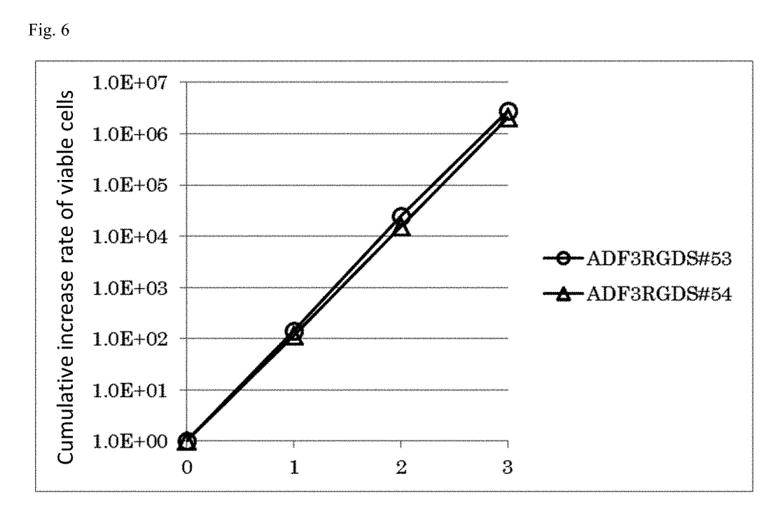

As shown in FIG. 6, when any of ADF3RGDS#53 and ADF3RGDS#54, which are fibroin-like proteins inserted with a cell adhesion sequence, was used for coating with the method of <1-1> and 20,000 or 25,000 viable cells were inoculated, culture was successfully carried out over a period of 3 weeks wherein subculture was carried out twice. The increase rate of viable cells was not changed during the culture, and that is, it was indicated that culture can be stably carried out without using a large amount of cells. After 3 weeks of the culture, cells were stained with an alkaline phosphatase staining kit (86-R; Sigma-Aldrich), and as a result, it was confirmed that cells were kept in undifferentiated state. This indicates that the product as described herein enables an iPS cell to proliferate while maintaining undifferentiated state thereof in a long-term culture.

As shown in Table 1, when any of ADF3RGDS#60, ADF3RGDS#65, ADF3RGDS#66, ADF3RGDS#68, ADF3RGDS#72, ADF3RGDS#73, ADF3RGDS#76, ADF3RGDS#77, and ADF3RGDS#78, which are fibroin-like proteins inserted with a cell adhesion sequence, was used for coating with the method of <1-1> or <1-2> and 13,000, 20,000, 50,000, or 10,0000 viable cells were inoculated, the increase rate of viable cells after 6 days or 7 days was equivalent to or larger than that observed with ADF3RGDS#2 or ADF3RGDS#3. Among them, ADF3RGDS#76 showed a high increase rate of viable cells, and it showed an increase rate of viable cells of 40 to 77 folds even when cells were subcultured at 7 days after the inoculation and then further cultured for 1 week.

TABLE 1

| Fibroin- | | | | |
|---|---|---|---|---|
| like protein | | | | |
| inserted with | | The number | The number | Increase |
| cell adhesion | | of inoculated | of measured | rate of |
| sequence | Coating | viable cells | viable cells | viable cells |
| ADF3RGDS#2 | Method of | 50,000 | 492,120 | 9.8 |
| | <1-2> | | | |
| ADF3RGDS#3 | Method of | 50,000 | 1,110,483 | 22.2 |
| | <1-2> | 20,000 | 153,150 | 7.7 |
| | | 13,000 | 68,318 | 5.3 |
| ADF3RGDS#60 | Method of | 50,000 | 791,543 | 15.8 |
| | <1-2> | | | |
| ADF3RGDS#65 | Method of | 100,000 | 2,092,500 | 20.9 |
| | <1-1> | | | |
| ADF3RGDS#66 | Method of | 50,000 | 578,917 | 11.6 |
| | <1-2> | | | |
| ADF3RGDS#68 | Method of | 50,000 | 1,474,619 | 29.5 |
| | <1-2> | | | |
| ADF3RGDS#72 | Method of | 50,000 | 2,185,599 | 43.7 |
| | <1-2> | | | |
| ADF3RGDS#73 | Method of | 50,000 | 2,045,218 | 40.9 |
| | <1-1> | | | |
| | Method of | 50,000 | 2,177,210 | 43.5 |
| | <1-2> | | | |
| ADF3RGDS#76 | Method of | 50,000 | 3,655,838 | 73.1 |
| | <1-2> | 20,000 | 1,982,046 | 99.1 |
| | | 13,000 | 1,045,257 | 80.4 |
| ADF3RGDS#77 | Method of | 50,000 | 2,924,998 | 58.5 |
| | <1-2> | 20,000 | 747,374 | 37.4 |
| ADF3RGDS#78 | Method of | 50,000 | 87,365 | 1.7 |
| | <1-2> | | | |

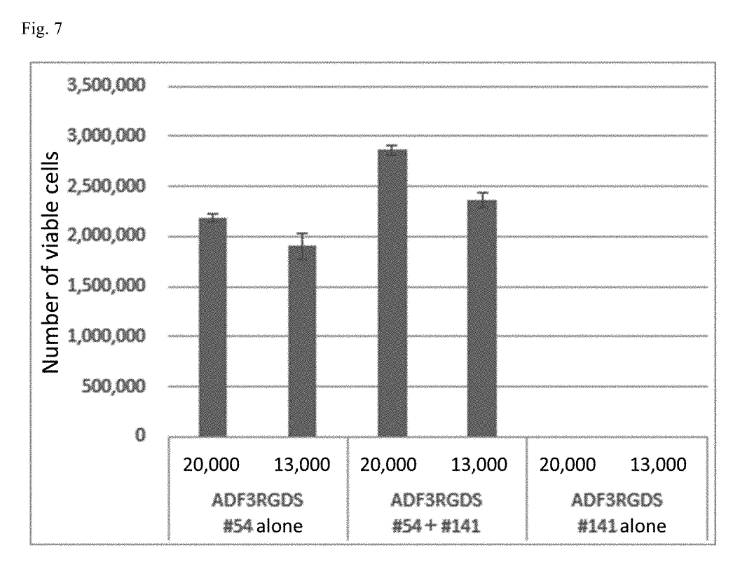

As shown in FIG. 7, when ADF3RGDS#54, which is a fibroin-like protein inserted with a cell adhesion sequence, was mixed with ADF3RGDS#141, which is a fibroin-like protein inserted with a heparin-binding sequence, and coated on the cell culture plate with the method of <1-2>, and 13,000 or 20,000 viable cells were inoculated, the number of viable cells after 6 days was increased by 31% or 24% as compared with that observed when ADF3RGDS#54 was solely used for coating and cell culture. When ADF3RGDS#141 was solely used for coating and cell culture, adhesion or proliferation of cells was not observed. This indicates that the scaffold function for an iPS cell of the product as described herein is increased by combined use of the product as described herein with a protein other than the product as described herein.

<Explanation of Sequence Listing>

SEQ ID NO: 1, Nucleotide sequence of fibroin-like protein gene ADF3

SEQ ID NO: 2, Amino acid sequence of fibroin-like protein ADF3

SEQ ID NO: 3, Nucleotide sequence of cell adhesion sequence-inserted fibroin-like protein gene ADF3RGDS#2

SEQ ID NO: 4, Amino acid sequence of cell adhesion sequence-inserted fibroin-like protein ADF3RGDS#2

SEQ ID NO: 5, Nucleotide sequence of cell adhesion sequence-inserted fibroin-like protein gene ADF3RGDS#3

SEQ ID NO: 6, Amino acid sequence of cell adhesion sequence-inserted fibroin-like protein ADF3RGDS#3

SEQ ID NOS: 7 to 10, Amino acid sequences of regions satisfying the requirement(s) of the Ala-rich region

SEQ ID NOS: 11 to 21, Amino acid sequences of motifs contained in regions satisfying the requirement(s) of the Ala-non-rich region

SEQ ID NOS: 22 to 25, Cell adhesion sequences containing RGD

SEQ ID NO: 26, Nucleotide sequence of cell adhesion sequence-inserted fibroin-like protein gene ADF3RGDS#53

SEQ ID NO: 27, Amino acid sequence of cell adhesion sequence-inserted fibroin-like protein ADF3RGDS#53

SEQ ID NO: 28, Nucleotide sequence of cell adhesion sequence-inserted fibroin-like protein gene ADF3RGDS#54

SEQ ID NO: 29, Amino acid sequence of cell adhesion sequence-inserted fibroin-like protein ADF3RGDS#54

SEQ ID NOS: 30 and 31, Cell adhesion sequences containing RGD

SEQ ID NO: 32, Amino acid sequence containing cell adhesion sequences containing RGD

SEQ ID NO: 33, Amino acid sequence consisting of two mutually-linked units of cell adhesion sequence containing RGD

SEQ ID NO: 34, Heparin-binding sequence

SEQ ID NO: 35, Nucleotide sequence of cell adhesion sequence-inserted fibroin-like protein gene ADF3RGDS#60

SEQ ID NO: 36, Amino acid sequence of cell adhesion sequence-inserted fibroin-like protein ADF3RGDS#60

SEQ ID NO: 37, Nucleotide sequence of cell adhesion sequence-inserted fibroin-like protein gene ADF3RGDS#65

SEQ ID NO: 38, Amino acid sequence of cell adhesion sequence-inserted fibroin-like protein ADF3RGDS#65

SEQ ID NO: 39, Nucleotide sequence of cell adhesion sequence-inserted fibroin-like protein gene ADF3RGDS#68

SEQ ID NO: 40, Amino acid sequence of cell adhesion sequence-inserted fibroin-like protein ADF3RGDS#68

SEQ ID NO: 41, Nucleotide sequence of cell adhesion sequence-inserted fibroin-like protein gene ADF3RGDS#72

SEQ ID NO: 42, Amino acid sequence of cell adhesion sequence-inserted fibroin-like protein ADF3RGDS#72

SEQ ID NO: 43, Nucleotide sequence of cell adhesion sequence-inserted fibroin-like protein gene ADF3RGDS#73

SEQ ID NO: 44, Amino acid sequence of cell adhesion sequence-inserted fibroin-like protein ADF3RGDS#73

SEQ ID NO: 45, Nucleotide sequence of cell adhesion sequence-inserted fibroin-like protein gene ADF3RGDS#76

SEQ ID NO: 46, Amino acid sequence of cell adhesion sequence-inserted fibroin-like protein ADF3RGDS#76

SEQ ID NO: 47, Nucleotide sequence of cell adhesion sequence-inserted fibroin-like protein gene ADF3RGDS#77

SEQ ID NO: 48, Amino acid sequence of cell adhesion sequence-inserted fibroin-like protein ADF3RGDS#77

SEQ ID NO: 49, Nucleotide sequence of heparin-binding sequence-inserted fibroin-like protein gene ADF3RGDS#141

SEQ ID NO: 50, Amino acid sequence of heparin-binding sequence-inserted fibroin-like protein ADF3RGDS#141

SEQ ID NO: 51, Nucleotide sequence of cell adhesion sequence-inserted fibroin-like protein gene ADF3RGDS#66

SEQ ID NO: 52, Amino acid sequence of cell adhesion sequence-inserted fibroin-like protein ADF3RGDS#66

SEQ ID NO: 53, Nucleotide sequence of cell adhesion sequence-inserted fibroin-like protein gene ADF3RGDS#78

SEQ ID NO: 54, Amino acid sequence of cell adhesion sequence-inserted fibroin-like protein ADF3RGDS#78

### INDUSTRIAL APPLICABILITY

According to the present invention, cells such as stem cells can be efficiently cultured.

1. A protein comprising a repetitive structure and a cell adhesion sequence,
wherein the repetitive structure consists of a repeating unit that repeats 3 to 15 times,
wherein each repeating unit consists of an Ala-rich region and an Ala-non-rich region mutually linked via a linkage site,
wherein each Ala-rich region consists of an amino acid sequence having a length of 8 to 54 residues,
wherein the ratio of Ala residues in each Ala-rich region is 30% or more,
wherein each Ala-rich region contains at least one Ala residue for every four consecutive amino acid residues,
wherein each Ala-non-rich region consists of an amino acid sequence having a length of 4 residues or longer,
wherein the ratio of Ala residues in each Ala-non-rich region is 20% or less,
wherein one or more of said repeating units in the repetitive structure comprises the cell adhesion sequence, and
wherein each cell adhesion sequence consists of an amino acid sequence comprising Arg-Gly-Asp and has a length of 3 to 18 residues.

2. The protein according to claim 1, wherein said cell adhesion sequence is present in two or more repeating units in the repetitive structure.

3. The protein according to claim 1, wherein the total number of said cell adhesion sequence that is present in the protein is 1 to 50.

4. The protein according to claim 1, wherein each cell adhesion sequence is present in the Ala-non-rich region, or in the linkage site between the Ala-rich region and the Ala-non-rich region

5. The protein according to claim 1, wherein each cell adhesion sequence consists of an amino acid sequence selected from the group consisting of:
(a) the amino acid sequence of SEQ ID NO: 22, 23, 24, 25, 30, or 31;
(b) the amino acid sequence of SEQ ID NO: 22, 23, 24, 25, 30, or 31, but wherein said sequence includes substitution, deletion, insertion, and/or addition of 1 to 3 amino acid residues at position(s) other than Arg-Gly-Asp; and
(c) an amino acid sequence having an identity of 80% or higher to the amino acid sequence of SEQ ID NO: 22, 23, 24, 25, 30, or 31, provided that Arg-Gly-Asp is conserved.

6. The protein according to claim 1, wherein each Ala-rich region consists of an amino acid sequence having a length of 8 to 15 residues.

7. The protein according to claim 1, wherein each Ala-rich region consists of an amino acid sequence having a length of 9 residues.

8. The protein according to claim 1, wherein each Ala-rich region contains one or more Gly residues and one or more Ser residues.

9. The protein according to claim 1, wherein the ratio of Ala residue in each Ala-rich region is 50% or more.

10. The protein according to claim 1, wherein the ratio of Ala, Gly, and Ser residues in each Ala-rich region is 90% or more in total.

11. The protein according to claim 1, wherein said Ala-rich region consists of an amino acid sequence selected from the group consisting of:
(a) the amino acid sequence of SEQ ID NO: 7, 8, 9, or 10;
(b) the amino acid sequence of SEQ ID NO: 7, 8, 9, or 10, but wherein said sequence includes substitution, deletion, insertion, and/or addition of 1 to 3 amino acid residues; and
(c) an amino acid sequence having an identity of 80% or higher to the amino acid sequence of SEQ ID NO: 7, 8, 9, or 10.

12. The protein according to claim 1, wherein each Ala-non-rich region consists of an amino acid sequence having a length of 15 to 100 residues.

13. The protein according to claim 1, wherein the ratio of Ala residue in each Ala-non-rich region is 5% or less.

14. The protein according to claim 1, wherein the ratio of Gly, Ser, Gln, Pro, and Tyr residues in each Ala-non-rich region is 90% or more in total.

15. The protein according to claim 1, wherein each Ala-non-rich region comprises an amino acid sequence selected from the group consisting of:
(a) one or more of the amino acid sequences of SEQ ID NOS: 11 to 21;
(b) one or more of the amino acid sequences of SEQ ID NOS: 11 to 21, but wherein said sequence includes substitution, deletion, insertion, and/or addition of 1 to 3 amino acid residues; and
(c) one or more amino acid sequences having an identity of 80% or higher to the amino acid sequences of SEQ ID NOS:11 to 21.

16. The protein according to claim 1, wherein each Ala-non-rich region comprises an amino acid sequence selected from the group consisting of:
(a) the amino acid sequence of amino acid numbers of 62 to 91, 101 to 120, 130 to 151, 161 to 185, 195 to 218, 228 to 257, 267 to 291, 301 to 325, 335 to 369, 379 to 438, 448 to 497, or 507 to 536 of SEQ ID NO: 2, or a partial sequence thereof;
(b) the amino acid sequence of amino acid numbers of 62 to 91, 101 to 120, 130 to 151, 161 to 185, 195 to 218, 228 to 257, 267 to 291, 301 to 325, 335 to 369, 379 to 438, 448 to 497, or 507 to 536 of SEQ ID NO: 2, or a partial sequence thereof, but wherein said sequence includes substitution, deletion, insertion, and/or addition of 1 to 10 amino acid residues; and
(c) an amino acid sequence having an identity of 90% or higher to the amino acid sequence of amino acid numbers of 62 to 91, 101 to 120, 130 to 151, 161 to 185, 195 to 218, 228 to 257, 267 to 291, 301 to 325, 335 to 369, 379 to 438, 448 to 497, or 507 to 536 of SEQ ID NO: 2, or a partial sequence thereof.

17. A composition comprising the protein according to claim 1.

18. An apparatus comprising the protein according to claim 1.

19. A method for producing a cultured cell, the method comprising culturing a cell by using the apparatus according to claim 18.

20. A gene encoding the protein according to claim 1.

21. A vector comprising the gene according to claim 20.

22. A host comprising the gene according to claim 20.
